(19) United States
(12) Patent Application Publication
(10) Pub. No.: US 2005/0143295 A1
(43) Pub. Date: Jun. 30, 2005

(54) Methods for treating drug addiction

(75) Inventors: John R. Walker, Solana Beach, CA (US); David W. Self, Dallas, CA (US); Kyle J. Frantz, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 10/680,806

(22) Filed: Oct. 6, 2003

Related U.S. Application Data
(63) Continuation of application No. PCT/US02/11094, filed on Apr. 4, 2002.
(60) Provisional application No. 60/281,440, filed on Apr. 4, 2001.

(51) Int. Cl. A61K 38/17; A61K 48/00

(52) U.S. Cl. 514/12; 514/44

(57) ABSTRACT

This invention describes gene targets for the development of therapeutics to treat drug addiction. Animal models of drug craving and relapse have been developed and used to find gene expression changes in key brain regions implicated in cocaine addiction. The genes whose expression levels are altered serve as pharmacological targets with the purpose of preventing or inhibiting cocaine craving and relapse in human cocaine addicts.

### CROSS-REFERENCE TO RELATED APPLICATIONS

This application is a National Stage of US application number PCT/US02/11094, filed Apr. 4, 2002; said application claims the benefit under 35 USC § 119(e) of U.S. provisional application No. 60/281,440 filed Apr. 4, 2001. The aforementioned applications are incorporated herein by reference for all purposes.

### BACKGROUND OF THE INVENTION

1. Field of the Invention

The present invention relates to the identification of differentially expressed genes in the brain that are involved in behavior associated with cocaine addiction. More particularly, the present invention relates to methods of identifying and using candidate agents to treat cocaine addiction based upon these genes.

2. Description of the Related Art

Drug and alcohol addictions are mental illnesses that exact an enormous social and economic cost from society. Although biomedical research has made tremendous advances in our understanding of how drugs affect the brain, very little of this information has translated into effective treatment strategies. This problem is particularly troublesome for cocaine addiction, where no effective treatments currently exist. Although many cocaine addicts can abstain from drug use for short periods of time, relapse rates at longer periods of abstinence are remarkably high, sometimes exceeding 90% (see Leshner, “Addiction Is a Brain Disease, and It Matters”, Science, Vol. 278, pp. 45-47 (1997)).

Progress in treating cocaine addiction has been hampered by the failure of animal models to target the primary behavioral disturbance, i.e., the increased propensity for relapse following prolonged periods of abstinence. Recently, the realization of this problem has led investigators to develop new animal models of drug craving in attempts to understand the underlying neurobiological mechanisms that trigger relapse to drug-seeking behavior, and to develop more effective treatment. In these studies, laboratory measures of “cocaine-seeking behavior” provide an objective measure of operant events such as lever-press responses that represent approach behavior analogous to relapse. In these studies, the level of drug-seeking behavior is indicated by the amount of effort (lever-pressing) exerted by animals to self-administer the drug. Importantly, this cocaine-seeking behavior is tested in the absence of drug reinforcement, because the reinforcing and rate-limiting effects of drugs can obscure the true incentive motivational state of the animals. Cocaine-seeking behavior can be measured by the magnitude and persistence of drug-paired lever responding during extinction testing, and by “reinstatement” of this responding following extinction. Either of these measures are thought to reflect the propensity for relapse in humans. Another advantage of these paradigms is that they can be tested during prolonged periods of forced abstinence. In contrast, subjective measures of drug craving in humans can be confounded by the subjective nature of self reports, and contextual differences between laboratory settings and the environment where humans routinely take drugs (see Tiffany et al., “The Development of a Cocaine Craving Questionnaire”, Drug Alcohol Depend., Vol. 34, pp. 19-28 (1993)).

Generally, there are only three stimuli known to reinstate drug-seeking behavior in animals following extinction of drug-self-administration. These stimuli consist of drug-associated (conditioned) cues, stress and low “priming” doses of the self-administered drug itself (for review, see Self et al., “Relapse to Drug Seeking: Neural and Molecular Mechanisms”, Drug Alcohol Depend., Vol. 51, pp. 49-60 (1998)). Since all three of these stimuli also trigger drug craving in human drug abusers (see Jaffe et al., “Cocaine-Induced Cocaine Craving”, Psychopharmacology, Vol. 97, pp. 59-64 (1989); Robbins et al., “Relationships Among Physiological and Self-Report Responses Produced by Cocaine-Related Cues”, Addictive, Vol. 22, pp. 157-167 (1997); and Sinha et al., “Stress Induced Craving and Stress Response in Cocaine Dependent Individuals”, Pschyopharmacology, Vol. 142, pp. 343-351 (1999) reinstatement of drug-seeking behavior in animals may represent a valid model of human drug craving. One caveat is that human drug addicts rarely, if ever, experience extinction conditions prior to relapse, but the striking concordance of reinstating stimuli in animals, and triggers of craving in humans, suggests that similar neurobiological processes are involved in both reinstatement and craving.

FIG. 1 depicts some of the primary pathways whereby stress, priming injections of drugs, and drug-associated cues are thought to induce relapse to drug-seeking behavior based on an evolving literature. There is a growing evidence that these stimuli all induce relapse, at least in part, by their ability to elevate dopamine levels in the nucleus accumbens (NAc). Thus, the NAc may be a critical neural substrate for relapse to drug seeking, in addition to its well-characterized role in drug reward. For example, abused drugs which elevate NAc dopamine levels also reinstate cocaine- and heroin-seeking behavior, while abused drugs like barbiturates that do not elevate NAc dopamine levels also fail to reinstate this behavior (reviewed by Self et al., supra). Similarly, infusion of drugs into brain regions where they activate NAc dopamine release reinstates cocaine- and heroin-seeking behavior, where infusion into regions where they do not is without effect.

Although it has not been clearly resolved, cue- and stress-induced reinstatement of drug-seeking behavior may involve both dopamine-dependent and dopamine-independent neural substrates (reviewed by Self et al., supra). An area of excitatory convergence is the NAc, where excitatory inputs from the prefrontal cortex (PfC), basolateral amygdala (BLA), and subiculum innervate medium spiny neurons receiving dopamine inputs from the ventral tegmental area (VTA). Excitatory neurotransmission in the NAc also has been implicated in reinstatement of cocaine-seeking behavior (see Cornish, et al., “A Role for Nucleus Accumbens Glutamate Transmission in the Relapse to Cocaine-Seeking Behavior”, Neuroscience, Vol. 93, pp. 1359-1367 (1999)). Together, these brain regions all form a complex circuit with primary sites of convergence in both the VTA and NAc of the mesolimbic dopamine system, as depicted in FIG. 1.

These studies highlight new and important information on the neural mechanisms of drug craving and relapse to drug seeking. Given that drug-seeking and drug craving can persist (or increase) despite long periods of abstinence, many current theories suggest that relatively long-term neuroadaptations in limbic brain regions associated with drug-seeking behavior underlie the propensity for relapse in addicted individuals. Most of these theories focus on pharmacological neuroadaptations directly produced by repeated drug exposure, leading to the phenomena of tolerance and sensitization (see Koob et al., “Drug Abuse: Hedonic Homeostatic Dysregulation”, Science, Vol. 278, pp. 52-58 (1997) and Nestler et al., “Molecular and Cellular Basis of Addiction”, Science, Vol. 278, pp. 58-63 (1997)). However, there is little evidence that most neuroadaptations persist during prolonged periods of abstinence (see White et al., “Neuroadaptions Involved in Amphetamine and Cocaine Addiction”, Drug Alc. Dep., Vol. 51, pp. 141-153 (1998)), and, thus, they cannot fully account for the propensity for relapse at these later time points. A major gap in our current knowledge is identifying stable neuroadaptations that underlie persistent drug craving in prolonged abstinence.

Recently, a behavioral paradigm in rats has been developed that models persistent craving for cocaine during prolonged abstinence. In fact, rats actually show increased levels of cocaine-seeking behavior as abstinence proceeds, a phenomenon also recently reported by Neisewander and colleagues (see Tran-Nguyen et al., “Time-Dependent Changes in Cocaine-Seeking Behavior and Extracellular Dopamine Levels in The Amygdala During Cocaine Withdrawal”, Neuropsychopharmacology, Vol. 19, pp. 48-59 (1998)). In this model, the level of cocaine-seeking behavior progressively increases from 1-6 weeks of forced abstinence from cocaine self-administration. The model is referred to as the “Cocaine Abstinence Effect”, and is thought to reflect time-dependent increases in cocaine craving that lead to increased relapse rates during prolonged abstinence. The model also represents the phenomenon of incentive sensitization, whereby drug-associated stimuli (environmental context, conditioned cues) show enhanced ability to stimulate craving as abstinence proceeds (see Robinson et al., “The Neural Basis of Drug Craving: An Incentive-Sensitization Theory of Addiction”, Brain Res. Rev., Vol. 18, pp. 247-291 (1993)).

In this model, rats are allowed to acquire intravenous cocaine self-administration on a fixed ratio 1:time-out 15-second schedule of reinforcement for 4 hours/day. Following 12 days of cocaine self-administration, different periods of forced abstinence are imposed whereby animals remain in their home cages, and are not allowed access to the self-administration test chambers. After a given period of abstinence, the rats are returned to the self-administration chambers, and the degree of drug-seeking behavior is measured by the number of non-reinforced responses at the drug-paired lever during extinction testing. FIG. 2 shows that cocaine-seeking behavior is approximately tripled when rats are returned to the test chambers during the third and sixth week of abstinence, relative to rats returned during their first week of abstinence. Six weeks of abstinence also produces more persistent cocaine-seeking behavior over the first few days of testing. By the sixth day of extinction testing, all three groups have extinguished to similar levels.

FIG. 3A shows time-dependent changes in the initial level of cocaine-seeking behavior when rats are first returned to the self-administration test chambers following forced abstinence. Rats tested after 2 and 5 weeks of forced abstinence exhibit 5- to 6-fold greater levels of drug-seeking behavior than at 1 day of abstinence. At the end of extinction testing, rats were tested for cue-induced reinstatement of cocaine-seeking behavior. In this test, cues specifically associated with the 10-second cocaine infusions during self-administration (house light off; lever cue light on; pump noise, vehicle infusion) were presented every 2 minutes for the final hour of the extinction/reinstatement test session. FIG. 3B shows that the cocaine abstinence effect is still evident following extinction testing, but only in the group tested during their sixth week of forced abstinence. Thus, cues specifically associated with cocaine infusions during self-administration induced greater reinstatement of responding at 6 weeks of abstinence than at 1 week of abstinence. Moreover, extinction training failed to completely reverse the Cocaine Abstinence Effect in this 6-week group, although cue-induced reinstatement at 3 weeks abstinence failed to differ as in extinction testing.

The Cocaine Abstinence Effect suggests that the incentive motivational effects of the drug-paired environment (extinction), and cocaine-associated cues (reinstatement), gain motivational salience with prolonged abstinence from cocaine. In contrast, pharmacological models of drug addiction and dependence suggest that drug craving would be maximal during early abstinence periods, when withdrawal symptoms also are maximal, and diminish as withdrawal effects wane over time (see Koob et al., supra).

The model closely parallels a similar effect of prolonged abstinence from chronic alcohol consumption known as the “alcohol deprivation effect” (see Sinclair, “The Alcohol-Deprivation Effect. Influence of various factors.”, Quarterly Journal of Studies on Alcohol, Vol. 33, pp. 769-782 (1972)), although it differs by measuring drug-seeking behavior rather than drug intake. This feature represents an important advantage over models of drug intake, because acute effects of drugs following abstinence could obscure certain biochemical measures that correlate with time-dependent increases in cocaine-seeking, and the response rate-limiting effects of drugs could alter behavioral measures of drug seeking. As cited above, at least one other group has published the phenomenon of time-dependent increases in cocaine seeking using similar (2 and 4 weeks) periods of forced abstinence (see Tran-Nguyen et al., supra). This study found that the behavioral effects also were associated with increased basal dopamine levels in the central nucleus (CeA) of the amygdala, and greater increases in dopamine release when animals were first returned to the self-administration chambers during extinction testing.

The “Cocaine Abstinence Effect” animal model is particularly useful in understanding the underlying biochemical neuroadaptations that trigger relapse to drug-seeking behavior. Accordingly, the use of this model to identify changes in gene expression that coincide with time-dependent increases in cocaine-seeking behavior and extinction training in rats, would aid in identifying potential therapeutic targets and therapeutic agents for use in treating cocaine addiction.

### SUMMARY OF THE INVENTION

The present invention is based on the identification of genes found in particular brain regions of rats that are modulated by behavior associated with cocaine addiction and extinction training. The genes have been identified by using a behavior animal model of cocaine addiction combined with oligonucleotide array profiling techniques. In particular, the present invention is directed to methods for inhibiting behavior associated with cocaine addiction in a subject such as a mammal suffering from cocaine addiction, and methods for identifying candidate agents useful in inhibiting behavior associated with cocaine addiction, using these genes.

In some embodiments, the invention provides methods for inhibiting addiction-related behavior in a subject suffering from cocaine addiction. These methods involve administering to the subject a therapeutically effective amount of a therapeutic agent which has the ability to modulate the level of activity of a polypeptide encoded by at least one gene identified in one or more of Tables 1-15. The activity of the polypeptide can be modulated by, for example, increasing or decreasing the level of expression of a gene that encodes the polypeptide, the level at which a transcript is translated or maintained in a cell, or by increasing or decreasing the enzymatic activity, binding ability, or other property of the polypeptide itself.

The invention also provides methods of inhibiting addiction-related behavior in a subject suffering from cocaine addiction that involve administering to the subject a therapeutically effective amount of a therapeutic agent which has the ability to decrease transcription/translation of, or decrease the activity of a protein encoded by, at least one gene that encodes a polypeptide selected from the group consisting of hypertension-regulated vascular factor, myelin-associated basic protein, PB cadherin, calcitonin receptor, melanocortin 4 receptor, ALK-7 kinase, and retroposon.

Also provided are methods of inhibiting addiction-related behavior in a subject suffering from cocaine addiction that involve administering to the subject a therapeutically effective amount of an agonist that activates a protein selected from the group consisting of GABA-B receptor subunit gb2, cell adhesion-like molecule, bos taurus-like neuronal axonal protein, a polypeptide similar to mouse chemokine-like factor, FRA-2, a protein similar to human oxygen-regulated protein, a protein similar to mouse mrg1 protein, pentraxin, malic enzyme, olfactomedin-related protein, arc-growth factor, protein tyrosine phosphatase, krox, neuritin, microtubule-associated protein 2d, and CB1 cannabinoid receptor.

Another aspect of the invention provides methods for identifying an agent to be tested for an ability to prevent or inhibit cocaine addiction-related behavior. These methods can involve: a) combining in a reaction mixture a candidate agent with a protein encoded by a gene identified in Tables 1-15; and b) determining whether the candidate agent binds to and/or modulates activity of the protein.

In some embodiments, these methods can further involve adding to the reaction mixture a competitor molecule that competes with binding of the candidate agent to the protein, either prior to or subsequent to combining the protein with the candidate agent.

In other embodiments, the methods further involve: c) administering the candidate agent identified in b) to a cocaine-addicted subject or brain cells of a cocaine-addicted subject, wherein the cocaine-addicted subject is undergoing withdrawal; and d) determining a level of expression of at least one gene identified in Tables 1-15 in brain cells of the cocaine-addicted subject. The level of expression is compared to that observed in brain cells of a cocaine-addicted subject to which the candidate agent is not administered, wherein a change in expression level is indicative of the candidate having efficacy in preventing or inhibiting cocaine addiction-related behavior.

Still other embodiments involve: c) administering the candidate agent identified in b) to a cocaine-addicted subject that is undergoing withdrawal; and d) determining whether the withdrawal symptoms exhibited by the subject are reduced upon administration of the candidate agent.

Also provided by the invention are methods for identifying an agent to be tested for an ability to prevent or inhibit addiction related behavior. These methods involve: a) exposing a cocaine-addicted subject or brain cells of a cocaine-addicted subject to a candidate agent, wherein the cocaine-addicted subject is undergoing withdrawal; b) determining a level of expression of at least one gene in the cocaine-addicted subject or brain cells of the cocaine-addicted subject, wherein the at least one gene is identified in Tables 1-15; and c) comparing the level of expression of the gene in the cocaine-addicted subject or brain cells of the cocaine-addicted subject in the presence of the candidate agent with the level of expression of the gene in the cocaine-addicted subject or brain cells of the cocaine-addicted subject in the absence of the candidate agent. A reversal in the level of expression of the gene in cocaine-addicted subject or brain cells of the cocaine addicted subject in the presence of the candidate agent relative to the level of expression of the gene in the absence of the candidate agent indicates that the candidate agent is an agent to be tested for the ability to prevent or inhibit addiction related behavior.

The invention also provides methods for identifying an agent to be tested for an ability to prevent or inhibit cocaine addiction-related behavior. These methods involve:
a) contacting a brain tissue sample from each of a subject having a cocaine addiction-related behavior and a cocaine addiction-free subject;
b) detecting a level of expression of at least one gene in both tissue samples, wherein the gene encodes a polypeptide selected from the group consisting of hypertension-regulated vascular factor, myelin-associated basic protein, PB cadherin, calcitonin receptor, melanocortin 4 receptor, ALK-7 kinase and retroposon.
c) subtracting the level of expression of the gene in the sample obtained from the cocaine addiction-free subject from the level of expression of the gene in the sample obtained from the subject having cocaine addiction-related behavior to provide a first value;
d) administering a candidate agent to each of a subject having a cocaine addiction-related behavior and a cocaine addiction-free subject;
e) detecting a level of expression of at least one gene in both tissue samples obtained from the subjects treated with the candidate agent;
f) subtracting the level of expression of the at least one gene in the sample obtained from the treated cocaine addiction-free subject from the level of expression of the gene in the sample obtained from the treated subject having the cocaine addiction-related behavior to provide a second value; and
g) comparing the second value with the first value wherein a decreased second value relative to the first value is indicative of an agent to be tested for an ability to prevent or inhibit cocaine addiction-related behavior.

In some embodiments, the invention provides methods for identifying agents to be tested for an ability to prevent or inhibit cocaine addiction-related behavior that involve:
a) obtaining a brain tissue sample from each of a subject having a cocaine addiction-related behavior and a cocaine addiction-free subject;
b) detecting a level of expression of at least one gene in both tissue samples, wherein the gene encodes a polypeptide selected from the group consisting of GABA-B receptor subunit gb2, cell adhesion-like molecule, bos taurus-like neuronal axonal protein, similar to mouse chemokine-like factor, FRA-2, a polypeptide similar to human oxygen-regulated protein, a polypeptide similar to mouse mrg1 protein, pentraxin, malic enzyme, olfactomedin-related protein, arc-growth factor enriched in dendrites, protein tyrosine phosphatase, krox, neuritin, microtubule-associated protein 2d and CB1 cannabinoid receptor;
c) subtracting the level of expression of the gene in the sample obtained from the cocaine addiction-free subject from the level of expression of the gene of the sample obtained from the subject having cocaine addiction-related behavior to provide a first value;
d) administering a candidate agent to each of a subject having a cocaine addiction-related behavior and a cocaine addiction-free subject;
e) detecting a level of expression of the gene in both tissue samples obtained from the subjects treated with the candidate agent;
f) subtracting the level of expression of the gene in the sample obtained from the treated cocaine addiction-free subject from the level of expression of the gene in the sample obtained from the treated subject having the cocaine addiction-related behavior to provide a second value; and
g) comparing the second value with the first value wherein an increased second value relative to the first value is indicative of an agent to be tested for an abilty to prevent or inhibit cocaine addiction related behavior.

### BRIEF DESCRIPTION OF THE DRAWINGS

FIG. 1. Diagrammatic representation of the primary pathways through which stress, drugs of abuse and drug-associated conditioned stimuli are hypothesized to trigger drug craving and relapse to drug-seeking. Stress and conditioned stimuli can activate excitatory glutamatergic projections to the VTA from the PfC, amygdala (Amyg) and hippocampus (Hipp), while priming injections of drugs directly stimulate dopamine (DA) release in the NAc. In this sense, dopamine release in the NAc may be a may be a final common trigger of drug craving by all three stimuli. At the level of NAc neurons, dopamine from the VTA modulates direct excitatory signals from the PfC, Amyg and Hipp where complex spatio-temporal integration of relapse-related information occurs. Studies showing involvement of these brain regions in reinstatement of drug-seeking suggest that long-term changes in gene expression in these regions would alter the functionality of this circuitry, and could produce profound changes in reactivity to stimuli that trigger drug craving and relapse to drug-seeking (adapted from Self et al., supra).

FIG. 2. Time-dependent increases in drug-seeking behavior during forced abstinence from cocaine self-administration. Groups of rats (ns=8-25) were balanced such that each group averaged similar levels of cocaine intake on the last 3 days of self-administration testing (1.0 mg/kg/infusion in 4 hour test sessions during the dark cycle). Following different periods of forced abstinence rats were returned to the drug-paired environment, and non-reinforced responding at the drug-paired lever was measured during 6 daily 4-hour extinction tests. Rats tested during the third and sixth week of forced abstinence showed significantly greater levels of drug-seeking behavior during the first 2 extinction tests than rats tested during the first week of forced abstinence (*P<0.05; Fisher's LSD).

FIG. 3. The Cocaine Abstinence Effect is evident at both the beginning (A) and end (B) of extinction testing. Selective responding at the drug-paired, rather than inactive, lever reflects the level of effort exerted by animals to self-administer cocaine (i.e., drug-seeking behavior). The left panel (A) depicts non-reinforced responding during the first hour of the initial 4-hour extinction test in groups of animals with forced abstinence ranging from 1 day to 5 weeks. The initial level of spontaneous drug-seeking behavior more than tripled at 2 and 5 weeks of forced abstinence when compared to rats tested after 1 day of forced abstinence (***P≦0.001). Following extinction testing, the ability of cues associated with cocaine infusions (house light off; lever light on; pump noise, vehicle infusion) to induce relapse to drug-seeking behavior was measured (B). The cues were non-contingently delivered for 10 seconds every 2 minutes for 1 hour immediately following the final extinction test. The level of drug-seeking behavior during cue-induced relapse doubled at 6 weeks when compared to 1 week of forced abstinence (**P<0.01; Fisher's LSD; 3-4 non-responders/group were not included in relapse analysis). Note that baseline response rates in the 1-hour period preceding cue exposure were similar for all 3 groups of rats (mean group responses ranged from 5.4-9.2).

FIG. 4. Effects of extinction training on withdrawal-induced changes in gene expression following 1 week abstinence from 12 days (4 hours/day at 1.0 mg/kg/injection) of cocaine self-administration. Example GeneChip profiles of mRNAs from NAc shell tissue are shown for 2 genes differentially regulated during early withdrawal by extinction training. Expression of the retroviral derived rat brain retroposon gene is elevated 88% during withdrawal from cocaine self-administration, but decreased 49% in animals that underwent 4 hours/day of extinction training, when compared to control values (see Table 1). The CB1 cannabinoid receptor is reduced 53% during withdrawal from cocaine self-administration, but is normalized (19% increase relative to control values) in animals that experienced extinction training during withdrawal. The top row of highlighted boxes in each array contains several different oligonucleotide sequences (25 bases/each) spanning the target sequence, while the bottom row contains a 1 base mismatch in the same sequences.

FIG. 5. Time-course and overall experimental strategy to identify changes in gene expression produced by cocaine self-administration (SA) abstinence and extinction. Arrows denote the time of sacrifice and dissection of the NAc shell for analysis with gene expression profiling. Group I remained in their home cages during 1 week of abstinence. Groups II and IV underwent 1 week of extinction training 1 week prior to sacrifice. Not shown are three groups that simultaneously underwent saline self-administration and were sacrificed along with Groups I, II and IV.

FIG. 6. Diagrammatic representation of tissue punches of limbic brain regions collected from animals during 1 week abstinence from cocaine self-administration for oligonucleotide array analysis. A “half-moon” outer punch of NAc shell was collected with a 12-gauge tissue punch. Each punch was taken from chilled brain slices immediately following sacrifice. The anatomical plates illustrate the posterior side of each 1.2-1.5 mm thick brain slice. For the current study, only the NAc shell was used. Other brain regions shown were also dissected but will be used for later studies.

FIG. 7. GABA-B receptor subunit gb2 protein levels are increased by extinction training in the NAc shell as measured by Western Blot. Values are expressed as a percentage of the mean of the control group.

FIGS. 8-10. Cannabinoid receptor CB1 protein levels are increased by cocaine withdrawal in the NAc shell as measured by Western Blot. Three different bands specific for CB1 were detected and quantitated separately: FIG. 8, 70 kDa glycosylated species; FIG. 9, upper 50 kDa nonglycosylated species; and FIG. 10, lower 50 kDAa glycosylated species.

### DETAILED DESCRIPTION OF THE INVENTION

All patent applications, patents and literature references cited herein are hereby incorporated by reference in their entirety.

The present invention relates to the identification of genes that are up- or down-regulated in particular regions of the brain of rats undergoing cocaine withdrawal compared with rats that are free from cocaine addiction (control) as shown below (see Tables 1-16.

As used herein, the term “up-regulated” with respect to these genes means that the expression of these genes is higher in rats undergoing cocaine withdrawal compared with rats that are free from cocaine addition. Such up-regulation refers to at least about a two-fold change.

As used herein, the term “down-regulated” with respect to these genes means that the expression of these genes is lower in cocaine-addicted rats undergoing withdrawal compared with rats that are free from cocaine addiction. Such down-regulation refers to at least about a two-fold changed.

Importantly, as shown in Table 1 the up- or down-regulation of many of these genes observed in the brain tissue of cocaine-addicted rats undergoing withdrawal is reversed upon subjecting these rats to extinction training. These results indicate a causal relationship between extinction-induced neuroadaptations in these genes and the propensity for behavior associated with cocaine addiction, particularly cocaine-seeking behavior. Accordingly, these differentially expressed genes can form the basis for novel agents useful in the treatment of cocaine addiction and in reducing, inhibiting or preventing addiction-related behavior in individuals suffering from cocaine addiction. In addition, these differentially expressed genes can be utilized to identify agents that inhibit or prevent behavior associated with cocaine addiction. Gene expression is typically assessed about 1-2 weeks after withdrawal.

The complete sequences of the genes listed in Tables 1-15 are available from GenBank database using the assigned accession numbers (as in Table 1) or part of the probe set identification numbers which indicate the accession numbers of the genes. For example, Probe set identification number “rc_AA875032_at listed in Table 3 corresponds to GenBank Accession No. AA875032. The sequences of these genes in GenBank, and their probe identification and accession numbers are expressly incorporated herein by reference.

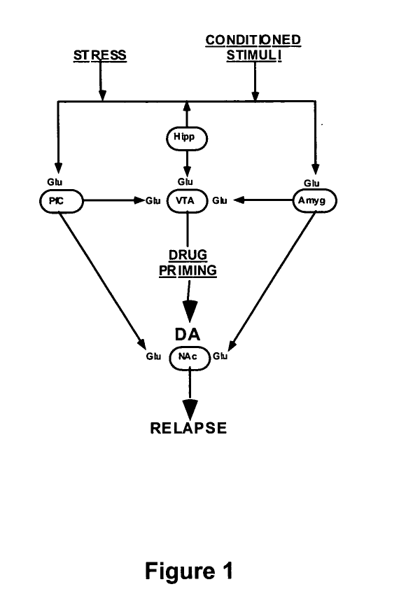

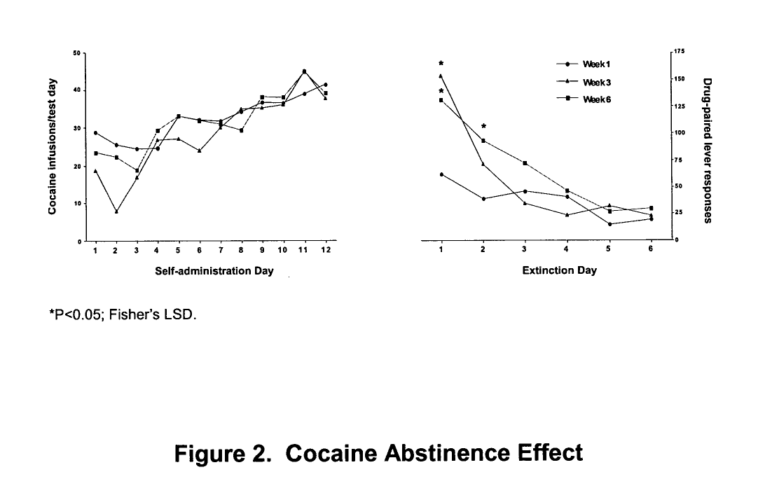

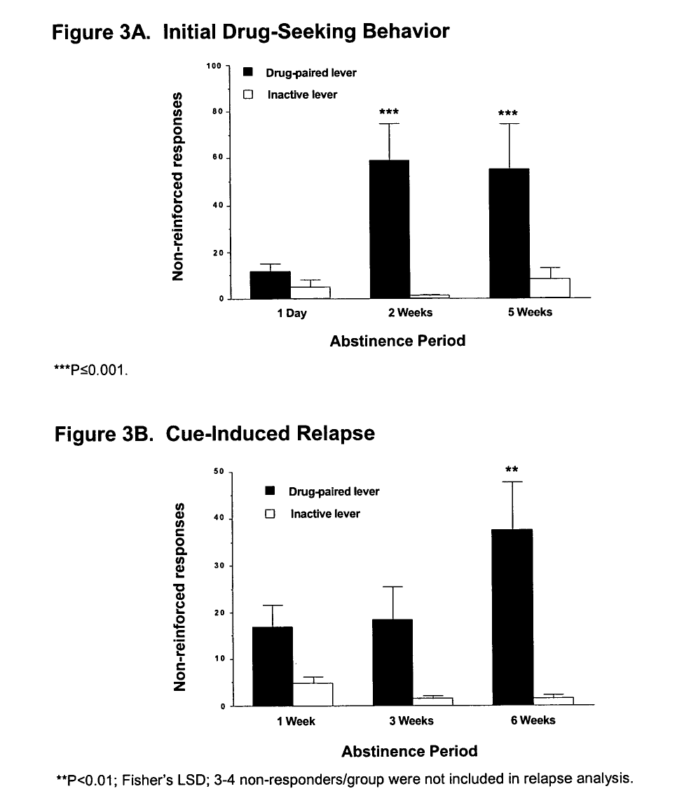

The brain regions where these genes are differentially expressed include the nucleus accumbens shell (Nac shell), the nucleus accumbens core (Nac core), the central nucleus of the amygdala (CeA), the ventral tegmental area (VTA) and the medial prefrontal cortex (mPFC). Evidence linking behavior associated with cocaine addiction to the aforementioned brain regions further support the involvement of the aforementioned genes expressed in these brain regions in such behavior. As stated above, although it has not been clearly resolved, cue- and stress-induced reinstatement of drug-seeking behavior may involve both dopamine-dependent and dopamine-independent neural substrates (reviewed by Self et al., supra). However, the basolateral amygdala (BLA), as well the CeA and related extended amygdala structures recently have been implicated in cue-and stress-induced reinstatement of drug-seeking behavior (see Meil et al, “Lesions of the Basolateral Amygdala a Bolish of the Ability of Drug Associated Cues to Reinstate Responding During Withdrawal from Self-Administered Cocaine”, Behav., Brain Res., Vol. 87, pp. 139-148 (1997); and Erb et al., “A Role for the Bed Nucleus of the Stria Terminalis, but Not the Amygdala, in the Effects of Corticotroopin-Releasing Factor on Stress-Induced Reinstatement of Cocaine Seeking”, J. Neurosci., Vol. 19, pp. C1-C6 (1999)). The CeA sends a direct excitatory projection to VTA neurons (see Gonzales et al., “Amydalonigral Pathway: An Anterograde Study in the Rat with Phaseolus Vulgaris Leucoagglutinin”, J. Comp. Neurol., Vol. 297, pp. 182-200 (1990); and Wallace et al., “Organization of Amygdaloid Projections to Brainstem Dopaminergic, Noradrenergic, and Adrenergic Cell Groups in the Rat”, Brain Res. Bull., Vol. 28, pp. 447-454 (1992)), which could mediate dopamine release in response to cues and stress. Other brain regions involved in relapse may include the PfC, where excitatory projections to dopamine neurons in the VTA activate dopamine release in the NAc (see Moghaddam, “Stress Preferentially Increases Extraneuronal Levels of Excitatory Amino Acids in the Prefrontal Cortex: Comparison to Hippocampus and Basal Ganglia”, J. Neurochem., Vol. 60, pp. 1650-1657 (1993); Taber, Das and Fibiger, “Cortical Regulation of Subcortical Dopamine Release: Mediation Via the Ventral Tegmental Area”, J. Neurochem., Vol. 65, pp. 1407-1410 (1995); and Karreman et al., “The Prefrontal Cortex Regulates the Basal Release of Dopamine in the Limbic Striatum: An Effect Mediated by Ventral Tegmental Area”, J. Neurochem., Vol. 66, pp. 589-598 (1996)). Similarly, recent studies have found that electrical stimulation of hippocampal-subiculuar outputs elevates dopamine levels in the NAc via excitatory inputs to the VTA (see Legault et al., “Chemical Stimulation of the Ventral Hippocampus Elevates Nucleus Accumbens Dopamine by Activating Dopaminergic Neurons of the Ventral Tegmental Area”, J. Neurosci., Vol. 20, pp. 1635-1642 (2000)), and also reinstates cocaine-seeking behavior (see Vorel et al., “Electrical Stimulation of Ventral Subiculum Induced Relapse to Cocaine Self-Administration”, Soc. Neurosci. Abstr., p. 2170 (1998). Another area of excitatory convergence is the NAc, where excitatory inputs from these the PfC, B1A and subiculum innervate medium spiny neurons receiving dopamine inputs from the VTA. Excitatory neurotransmission in the NAc also has been implicated in reinstatement of cocaine-seeking behavior (see Cornish et al., supra). Together, these brain regions all form a complex circuit with primary sites of convergence in both the VTA and NAc of the mesolimbic dopamine system, as depicted in FIG. 1.

Any selection of at least one of the genes listed in Tables 1-15 can be utilized as a therapeutic target for inhibiting or preventing behavior associated with cocaine addiction. Preferably at least one of the genes is identified in Tables 1, 5, 8, 11 and 14, and more preferably at least one gene is identified in Table 1. In particularly useful embodiments, a plurality of these genes, i.e. two or more, can be selected and their expression monitored simultaneously to provide expression profiles for use in various aspects. For example, expression profiles of these genes can provide valuable molecular tools for rapidly identifying agents that alter these expression profiles. Particularly preferred genes from Tables 1-15 that are useful as therapeutic targets include those listed in Table 16.

In one aspect, methods of treating addiction-related behavior in a subject, e.g., a human or animal, suffering from cocaine addiction are provided which involve preventing or inhibiting cocaine-addiction related behavior utilizing various therapeutics that modulate the transcription/translation of these differentially expressed genes or that modulate the activity of proteins encoded by these genes. As used herein, cocaine refers to cocaine itself and derivatives of cocaine, e.g., crack. As used herein the term “addiction-related behavior” refers to behavior resulting from cocaine use and is characterized by apparent total dependency on cocaine. Symptomatic of such behavior is (i) overwhelming involvement with the use of cocaine; (ii) the securing of its supply; and (iii) a high probability of relapse following withdrawal. For example, in cocaine users addiction-related behavior typically includes behavior associated with three stages of drug effects. In the first stage, acute intoxication, “binge”, is euphoric, marked by decreased anxiety, enhanced self-confidence and sexual appetite. In the second stage, the “crash” replaces the euphoric feeling with anxiety, fatigue, irritability and depression. The third stage, “anhedonia” is a time of limited ability to experience pleasure from normal activities and of craving for the euphoric effects of cocaine. In particularly useful embodiments, the cocaine-addiction related behavior is cocaine seeking. As used herein, cocaine seeking which is a behavior measured in cocaine-addicted animals such as rats is assumed to be analogous to the behavior, cocaine craving, that is observed in humans.

Examples of suitable therapeutic agents for inhibiting or preventing cocaine addiction-related behavior include, but are not limited to, antisense sequences, ribozymes, double-stranded RNAs, small inhibitory RNA (siRNA), agonists and antagonists as described in detail below.

As used herein, the term “antisense” refers to nucleotide sequences that are complementary to a portion of an RNA expression product of at least one of the disclosed genes. “Complementary” nucleotide sequences refer to nucleotide sequences that are capable of base-pairing according to the standard Watson-Crick complementarity rules. That is, purines will base-pair with pyrimidine to form combinations of guanine:cytosine and adenine:thymine in the case of DNA, or adenine:uracil in the case of RNA. Other less common bases, e.g., inosine, 5-methylcytosine, 6-methyladenine, hypoxanthine and others may be included in the hybridizing sequences and will not interfere with pairing.

When introduced into a host cell, antisense nucleotide sequences specifically hybridize with the cellular mRNA and/or genomic DNA corresponding to the gene(s) so as to inhibit expression of the encoded protein, e.g., by inhibiting transcription and/or translation within the cell.

The isolated nucleic acid molecule comprising the antisense nucleotide sequence can be delivered, e.g., as an expression vector, which when transcribed in the cell, produces RNA which is complementary to at least a unique portion of the encoded mRNA of the gene(s). Alternatively, the isolated nucleic acid molecule comprising the antisense nucleotide sequence is an oligonucleotide probe which is prepared ex vivo and, which, when introduced into the cell, results in inhibiting expression of the encoded protein by hybridizing with the mRNA and/or genomic sequences of the gene(s).

The oligonucleotide can include artificial internucleotide linkages which render the antisense molecule resistant to exonucleases and endonucleases, and thus are stable in the cell. Examples of modified nucleic acid molecules for use as antisense nucleotide sequences are phosphoramidate, phosporothioate and methylphosphonate analogs of DNA as described, e.g., in U.S. Pat. Nos. 5,176,996; 5,264,564; and 5,256,775. General approaches to preparing oligomers useful in antisense therapy are described, e.g., in Van der Krol., BioTechniques 6:958-976, 1988; and Stein et al., Cancer Res. 48:2659-2668, 1988.

Typical antisense approaches, involve the preparation of oligonucleotides, either DNA or RNA, that are complementary to the encoded mRNA of the gene. The antisense oligonucleotides will hybridize to the encoded mRNA of the gene and prevent translation. The capacity of the antisense nucleotide sequence to hybridize with the desired gene will depend on the degree of complementarity and the length of the antisense nucleotide sequence. Typically, as the length of the hybridizing nucleic acid increases, the more base mismatches with an RNA it may contain and still form a stable duplex or triplex. One skilled in the art can determine a tolerable degree of mismatch by use of conventional procedures to determine the melting point of the hybridized complexes.

Antisense oligonucleotides are preferably designed to be complementary to the 5′ end of the mRNA, e.g., the 5′untranslated sequence up to and including the regions complementary to the mRNA initiation site, i.e., AUG. However, oligonucleotide sequences that are complementary to the 3′ untranslated sequence of mRNA have also been shown to be effective at inhibiting translation of mRNAs as described e.g., in Wagner, Nature 372:333, 1994. While antisense oligonucleotides can be designed to be complementary to the mRNA coding regions, such oligonucleotides are less efficient inhibitors of translation.

Regardless of the mRNA region to which they hybridize, antisense oligonucleotides are generally from about 15 to about 25 nucleotides in length.

The antisense nucleotide can also comprise at least one modified base moiety, e.g., 3-methylcytosine, 5,-methylcytosine, 7-methylguanine, 5-fluorouracil, 5-bromouracil, and may also comprise at least one modified sugar moiety, e.g., rabinose, hexose, 2-fluorarabinose, and xylulose.

In another embodiment, the antisense nucleotide sequence is an alpha-anomeric nucleotide sequence. An alpha-anomeric nucleotide sequence forms specific double stranded hybrids with complementary RNA, in which, contrary to the usual beta-units, the strands run parallel to each other as described e.g., in Gautier et al., Nucl. Acids. Res. 15:6625-6641, 1987.

Antisense nucleotides can be delivered to cells which express the described genes in vivo by various techniques, e.g., injection directly into the prostate tissue site, entrapping the antisense nucleotide in a liposome, by administering modified antisense nucleotides which are targeted to the prostate cells by linking the antisense nucleotides to peptides or antibodies that specifically bind receptors or antigens expressed on the cell surface.

However, with the above-mentioned delivery methods, it may be difficult to attain intracellular concentrations sufficient to inhibit translation of endogenous mRNA. Accordingly, in a preferred embodiment, the nucleic acid comprising an antisense nucleotide sequence is placed under the transcriptional control of a promoter, i.e., a DNA sequence which is required to initiate transcription of the specific genes, to form an expression construct. The use of such a construct to transfect cells results in the transcription of sufficient amounts of single stranded RNAs to hybridize with the endogenous mRNAs of the described genes, thereby inhibiting translation of the encoded mRNA of the gene. For example, a vector can be introduced in vivo such that it is taken up by a cell and directs the transcription of the antisense nucleotide sequence. Such vectors can be constructed by standard recombinant technology methods. Typical expression vectors include bacterial plasmids or phage, such as those of the pUC or Bluescript.™ plasmid series, or viral vectors such as adenovirus, adeno-associated virus, herpes virus, vaccinia virus and retrovirus adapted for use in eukaryotic cells. Expression of the antisense nucleotide sequence can be achieved by any promoter known in the art to act in mammalian cells. Examples of such promoters include, but are not limited to, the promoter contained in the 3′ long terminal repeat of Rous sarcoma virsu as described, e.g., in Yamamoto et al., Cell 22: 787-797, 1980; the herpes thymidine kinase promoter as described e.g., in Wagner et al., Proc. Natl. Acad. Sci. U.S.A. 78:1441-1445, 1981; the SV40 early promoter region as described e.g., in Bernoist and Chambon, Nature 290:304-310, 1981; and the regulatory sequences of the metallothionein gene as described, e.g., in Brinster et al., Nature 296:39-42, 1982.

Ribozymes are RNA molecules that specifically cleave other single-stranded RNA in a manner similar to DNA restriction endonucleases. By modifying the nucleotide sequences encoding the RNAs, ribozymes can be synthesized to recognize specific nucleotide sequences in a molecule and cleave it as described, e.g., in Cech, J. Amer. Med. Assn. 260:3030, 1988. Accordingly, only mRNAs with specific sequences are cleaved and inactivated.

Two basic types of ribozymes include the “hammerhead”-type as described for example in Rossie et al. Pharmac. Ther. 50:245-254, 1991; and the hairpin ribozyme as described, e.g., in Hampel et al, Nucl. Acids Res. 18:299-304, 1999 and U.S. Pat. No. 5,254,678. Intracellular expression of hammerhead and hairpin ribozymes targeted to mRNA corresponding to at least one of the disclosed genes can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozyme sequences can be modified in essentially the same manner as described for antisense nucleotides, e.g., the ribozyme sequence can comprise a modified base moiety.

Double-stranded RNA, i.e., sense-antisense RNA, corresponding to at least one of the disclosed genes, can also be utilized to interfere with expression of at least one of the disclosed genes. Interference with the function and expression of endogenous genes by double-stranded RNA has been shown in various organisms such as C. elegans as described, e.g., in Fire et al., Nature 391:806-811, 1998; drosophilia as described, e.g., in Kennerdell et al., Cell 95(7):1017-26, 1998; and mouse embryos as described, e.g., in Wianni et al., Nat. Cell Biol. 2(2):70-5, 2000. Such double-stranded RNA can be synthesized by in vitro transcription of single-stranded RNA read from both directions of a template and in vitro annealing of sense and antisense RNA strands. Double-stranded RNA can also be synthesized from a cDNA vector construct in which the gene of interest is cloned in opposing orientations separated by an inverted repeat. Following cell transfection, the RNA is transcribed and the complementary strands reanneal. Double-stranded RNA corresponding to at least one of the disclosed genes could be introduced into a prostate cell by cell transfection of a construct such as that described above.

The term “antagonist” refers to a molecule which when bound to the protein encoded by the gene inhibits its activity. Antagonists can include, but are not limited to, peptides, proteins, carbohydrates and small molecules. In a particularly useful embodiment, the antagonist is an antibody specific for the protein expressed by the at least one gene.

The term “agonist” as used herein refers to any natural or synthetic molecule which, when bound to the expressed protein, increases or prolong the duration of the effect of the protein. Agonists can include proteins, nucleic acids, carbohydrates or any other molecules that bind to and modulate the effect of the protein.

In one embodiment, a method of inhibiting addiction-related behavior in a subject suffering from cocaine addiction is provided which comprises administering to the subject a therapeutically effective amount of a therapeutic agent which has the ability to modulate the transcription/translation of at least one gene or the activity of a protein encoded by the genes, wherein the at least one gene is identified in Tables 1, 2 and 4-15. In the case where the therapeutic agent is an antisense sequence, an isolated nucleic acid molecule encoding a ribozyme, or a double stranded RNA, such an agent modulates the transcription/translation of the gene. In the case wherein the therapeutic agent is an antagonist or agonist, such an agent modulates the activity of a protein encoded by the gene.

As used herein, the term “isolated” nucleic acid molecule means that the nucleic acid molecule is removed from its original environment (e.g., the natural environment if it is naturally occurring). For example, a naturallly occurring nucleic acid molecule is not isolated, but the same nucleic acid molecule, separated from some or all of the coexisting materials in the natural system, is isolated, even if subsequently reintroduced into the natural system. Such nucleic acid molecules could be part of a vector or part of a composition and still be isolated, in that such vector or composition is not part of its natural environment.

As used herein, the term “modulate” with respect to transcription/translation refers to the up-or down-regulation of transcription/translation of the gene, i.e., that is “modulate” includes either an increase or a decrease in expression of the at least one gene. The direction of modulation affected by the therapeutic agent depends on which gene is being modulated. For example, the calcitonin receptor gene is upregulated in the Nac Shell of cocaine-addicted rats during cocaine withdrawal. Accordingly, an antisense sequence, a ribozyme, or a double stranded RNA modulates expression of the calcitonin gene by blocking the “up-regulation” of expression of this gene or reversing or “down-regulating” the expression of this gene.

As used herein, the term “modulate” with respect to activity of a protein encoded by the gene, refers to an alteration, i.e., increase or decrease, in the activity of a protein encoded by the gene. For example, the gene encoding malic enzyme is down-regulated in Nac Shell of cocaine-addicted rats during cocaine withdrawal. Accordingly, an agonist that would increase the activity of the malic enzyme can aid in inhibiting addiction-related behavior.

In a preferred embodiment of the method for inhibiting or preventing cocaine-addiction related behavior, the at least one gene identified in Table 1 encodes a polypeptide selected from the group consisting of GABA-B receptor subunit gb2, myelin-associated basic protein, calcitonin receptor, Bos taurus-like neuronal axonal protein, FRA-2, a polypeptide similar to human oxygen-regulated protein, a polypeptide similar to mouse mrg 1 protein, pentraxin, olfactomedin-related protein, arc-growth factor (enriched in dendrites), protein tyrosine phosphatase, melanocortin 4 receptor, ALK-7 kinase, neuritin and CB1 cannabinoid receptor. More preferably, the at least one gene identified in Table 1 encodes GABA-B receptor subunit gb2, FRA-2 and CB1 cannabinoid receptor. In some embodiments of the method for inhibiting or preventing cocaine addiction-related behavior, the at least one gene identified in Table 1 does not encode melanocortin 4 receptor.

In another preferred embodiment of the method for inhibiting or preventing cocaine addiction-related behavior, the at least one gene is identified in Table 2.

In another preferred embodiment of the above method, the at least one gene is identified in Table 4, and more preferably encodes a polypeptide selected from the group consisting of GABAB receptor 1d, tyrosine kinase receptor RET and Neurodap-1.

In another preferred embodiment of this method, the at least one gene is identified in Table 5, and more preferably encodes a polypeptide selected from the group consisting of inhibin alpha-subunit and vesicular transport factor.

In another preferred embodiment of this method, the at least one gene is identified in Table 6, and more preferably encodes a polypeptide selected from the group consisting of GABAB receptor 1c and phosphatidylinositol 4-kinase.

In another preferred embodiment of this method, the at least one gene is identified in Table 7 and more preferably encodes a polypeptide selected from the group consisting of somatostain-14 and kainate receptor submit (ka2).

In another preferred embodiment of this method, the at least one gene is identified in Table 8, and more preferably encodes a polypeptide selected from the group consisting of melanocortin-3 receptor, somatostatin, metabotropic glutamate receptor 3, NCAM polypeptide and synaptic SAPAP-interacting protein.

In another preferred embodiment of this method, the at least one gene is identified in Table 9, and more preferably encodes calpastatin.

In another preferred embodiment of this method, the at least one gene is identified in Table 10, and more preferably encodes a polypeptide selected from the group consisting of RAC protein kinase alpha, alpha-2B-adrenergic receptor and SNAP-25A.

In another preferred embodiment of this method, the at least one gene is identified in Table 11, and more preferably encodes a polypeptide selected from the group consisting of oxytosin/neurophysin, NMDAR2C and GABA-A receptor epsilon.

In another preferred embodiment of thiis method, the at least one gene is identified in Table 12, and preferably encodes a polypeptide selected from the group consisting of phosphodiesterase I, tyrosine phosphatase and dopamine transporter.

In yet another preferred embodiment of this method, the at least one gene is identified in Table 13, and preferably encodes synaptotagmin IV homolog.

In another useful embodiment of this method, the at least one gene is identified in Table 14, and preferably encodes a polypeptide selected from the group consisting of calmodulin, protein kinase rMNK2, phospholipase C-beta1b.

In another useful embodiment of this method, the at least one gene is identified in Table 15, and preferably encodes a polypeptide selected from the group consisting of phosphatidylinositol 4-kinase and protein-tyrosine-phosphatase.

A “therapeutically effective amount” of a therapeutic agent refers to a sufficient amount of the therapeutic agent to prevent or inhibit cocaine addiction-related behavior in a subject suffering from cocaine addiction. The determination of a therapeutically effective amount is well within the capability of those skilled in the art. For any therapeutic, the therapeutically effective dose can be estimated in animal models, usually mice, rats, rabbits, dogs or pigs. The animal model may also be used to determine the appropriate concentration range and route of administration. Such information can then be used to determine useful doses and routes for administration in humans.

Therapeutic efficacy and toxicity may be determined by standard pharmaceutical procedures in experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Antisense nucleotides, ribozymes, double-stranded RNAs, antagonists and agonists, and other therapeutic agents that exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the subject and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight and gender of the subject, diet, time and frequency of administration, drug combination(s), reaction sensitivities and tolerance/response to therapy.

Normal dosage amounts may vary from 0.1-100,000 mg, up to a total dose of about 1 g, depending upon the route of administration. Guidance as to particular dosages and methods of delivery is provided in the literature and generally available to practitioners in the art. Those skilled in the art will employ different formulations for nucleotides than for antagonists.

For therapeutic applications, the therapeutic agents are preferably administered as pharmaceutical compositions containing the therapeutic agent in combination with one or more pharmaceutically acceptable carriers. The compositions may be administered alone or in combination with at least one other agent, such as stabilizing compound, which may be administered in any sterile, biocompatible pharmaceutical carrier, including, but not limited to, saline, buffered saline, dextrose and water. The compositions may be administered to a subject, or in combination with other agents or drugs.

In another aspect, the present invention provides screening methods for identifying agents to be tested for the ability to inhibit or prevent cocaine addiction-related behavior. The screening methods are typically designed to find candidate agents that can interact, i.e., bind, to proteins encoded by these differentially expressed genes, and then these agents can be used in assays that ascertain the ability of the candidate agent to modify the activity of the protein. Such binding and activity assays can be performed in cell-free systems, e.g., in a reconstituted protein mixture or a cell membrane preparation, and in cells, particularly recombinant cells expressing the protein encoded by the gene. In particularly useful embodiments of these screening methods, candidate agents are screened in animal models for their ability to reverse, i.e., either increase or decrease, the expression of at least one of the disclosed genes that are upregulated or down regulated by cocaine withdrawal.

As used herein, the term “candidate agent” refers to any molecule that is capable of interacting, i.e., binding to, and/or increasing or decreasing the activity of, a protein encoded by one of the disclosed genes. The candidate agent can modify the structure of the encoded protein to thereby alter the activity of the protein. The candidate agent also refers to any molecule that is capable of increasing/decreasing the level of mRNA corresponding to or protein encoded by at least one of the disclosed genes. The candidate agent can be natural or synthetic molecules such as proteins or fragments thereof, antibodies, nucleic acid molecules, e.g., antisense nucleotides, ribozymes, double-stranded RNAs, organic and inorganic compounds and the like.

In one embodiment, cell-free assays for identifying such candidate agents comprise combining in a reaction mixture, i.e, a cell-free system or cell-based system, a candidate agent with a protein encoded by one of the disclosed genes in Tables 1-15 and determining the interaction, i.e., binding, of the candidate agent to the protein or modulation of the activity of the protein. In other embodiments, a fragment of the protein encoded by the disclosed gene can be combined with the candidate agent. Preferred proteins include those encoded by genes identified in Tables 1, 5, 8, 11 and 14. More preferred proteins are those encoded by the preferred listed genes for each of Tables 1, 2, and 4-15, and preferably Table 1 as described above in the methods for inhibiting addiction-related behavior. In some embodiments of this cell-free assay, the gene identified in Table 1 does not encode CB1 cannibinoid receptor.

In a particularly useful embodiment, the protein encoded by the disclosed gene or the candidate agent is immobilized to an insoluble support to facilitate separation of complexes of the protein/candidate agent from uncomplexed forms of the protein and automation of the assay. The insoluble support may be solid or porous and possess any shape. Examples of suitable solid supports include, but are not limited to, microtitre plates and arrays, micro-centrifuge tubes, test tubes, membranes and beads. Particularly useful methods of binding include, but are not limited to, the use of antibodies, direct binding to ionic supports, and chemical crosslinking. Subsequent to binding of the protein or agent to the support, unbound material is removed by washing.

In a preferred embodiment, the protein encoded by the gene is bound to the insoluble support, and the candidate agent is then added. Alternatively, the candidate agent is bound to the solid support and the protein encoded by the gene is added.

Determination of the binding of the candidate agent to the encoded protein can be carried out by standard methods. For example, the candidate agent can be labeled, and binding determined by, e.g, attaching the protein or fragment thereof to the insoluble support, adding the labeled candidate agent, washing off unbound candidate agent, and determining whether any label is bound to the support.

The term “labeled” means that the candidate agent or protein is either directly or indirectly labeled with a label to provide a detectable signal, e.g., enzymes, antibodies, radioisotopes, fluorescers, chemiluminescers, or specific binding molecule pairs such as biotin and streptavidin. For example, the protein can be biotinylated using biotin NHS(N-hydroxysuccinimide), using well-known techniques and immobilized in the well of streptavidin-coated plates.

Interaction (binding) between molecules can also be assessed by using real-time BIA (Biomolecular Interaction Analysis, Pharmacia Biosensor AB), which detects surface plasmon resonance, an optical phenomenon. Detection depends on changes in the mass concentration of mass macromolecules at the biospecific interface and does not require labeling of the molecules. In one useful embodiment, a library of candidate agents, such as organic compounds, can be immobilized on a sensor surface, e.g., a wall of a micro-flow cell. A solution containing the protein or functional fragment thereof is then continuously circulated over the sensor surface. An alteration in the resonance angle, as indicated on a signal recording, indicates the occurrence of an interaction. This technique is described in more detail in BIAtechnology Handbook by Pharmacia.

In another embodiment, the binding of the candidate agent to the protein encoded by the gene can be determined using competitive binding assays wherein a competitor, i.e., a substance known to bind to the encoded protein such as an antibody, ligand, peptide, etc., is combined with the encoded protein, either prior to or subsequent to combining the protein with the candidate agent. For example, the competitor can be added to the protein followed by the candidate agent. Displacement of the competitor indicates that the candidate agent is binding to the encoded protein. In this embodiment, the candidate agent or competitor can be labeled. Accordingly, if a labeled competitor is used, the presence of the label in the wash removed from the insoluble support, indicates displacement by the candidate agent. Alternatively, if the candidate agent is labeled, the presence of the label on the insoluble support indicates displacement of the competitor.

Cell-free assays can also be used to identify agents which interact with a protein encoded by one of the disclosed genes and modulate the activity of this protein. In one embodiment, the protein encoded by one of the disclosed genes is incubated with a candidate agent, such as an organic compound and the catalytic activity of the protein is determined.

In another aspect, a cell-based assay is provided for screening candidate agents that bind to a protein encoded by one of the disclosed genes. The method comprises providing a recombinant cell expressing a protein encoded by one of the genes identified in Tables 1-15, contacting the cell with a candidate agent; and determining the binding of the candidate agent to the protein. As used herein, the term “recombinant cell” refers to a cell that has been transfected by one of the disclosed genes, wherein the cell expresses the gene. The recombinant cell is preferably a mammalian cell, an insect cell, a xenopus cell or an oocyte. Cells used as controls include cells that are substantially identical to the recombinant cells, but do not express the proteins encoded by the disclosed genes. The binding of the candidate agent to the protein expressed by the cell can be determined by e.g., detecting a signal in the cell, e.g., alterations in second messengers which are sensitive to binding of the candidate agent. Such a recombinant cell further comprises a reporter gene operatively linked to a transcriptional control sequence which is responsive to an intracellular signal, i.e., a second messenger, transduced by interaction of the candidate agent with the protein expressed by the recombinant cell. For example, cyclic AMP accumulation induced by CB1 activation can be measured using a cyclic AMP response element (CRE) reporter assay. Candidate agents that enhance or suppress expression of the reporter interact with either CB1 or its signal transduction system.

The term “transcriptional control sequence” refers to DNA sequences, such as initiator sequences, enhancer sequences and promoter sequences, which induce, repress or ortherwise control the transcription of protein encoding nucleic acid sequence to which they are operatively linked. Upon induction of the transcriptional control sequence by the second messenger, the reporter gene is expressed thereby providing a quantifiable and detectable signal, e.g., color, fluorescence, luminescence, cell growth, drug resistance, etc., that determines binding of the candidate agent to the protein. Examples of such reporter genes include, but are not limited to, luciferase, alkaline phosphatase, chloramphenicol acetyl transferase and betagalactosidase. In some embodiments, the protein encoded by one of the genes identified in Table 1 is not CB1 cannabinoid receptor. In some embodiments, modulation of binding of the protein encoded by one of the disclosed genes to the candidate agent can be determined in the presence of a target protein or target peptide which is known to bind to the a protein encoded by one of the disclosed genes.

In yet another embodiment, the effect of a candidate agent on the transcription of one of the genes disclosed in Tables 1-15 is determined by transfection experiments using a reporter gene operatively linked to at least a portion of a transcriptional control sequence of a gene identified in Tables 1-15.

Assays based on animal models or cells obtained from such animals can also be used to identify agents which modulate the expression of a gene identified in Tables 1-15, that has undergone up- or down-regulation upon cocaine-withdrawal. Accordingly, in one embodiment, a method for identifying an agent to be tested for an ability to prevent or inhibit addiction related-behavior is provided which comprises:
a) exposing a cocaine-addicted subject or brain cells of a cocaine-addicted subject to a candidate agent, wherein the cocaine-addicted subject is undergoing withdrawal;
b) determining a level of expression of at least one gene in the cocaine-addicted subject or brain cells of the cocaine-addicted subject, wherein the at least one gene is identified in Tables 1-15; and
comparing the level of expression of the gene in both the cocaine-addicted subject or brain cells of the cocaine-addicted subject in the presence of the candidate agent with the level of expression of the gene in the cocaine-addicted subject or brain cells of the cocaine-addicted subject in the absence of the candidate agent, wherein a reversal in the level of expression of the gene in the cocaine-addicted subject or brain cells of the cocaine-addicted subject in the presence of the candidate agent relative to the level of expression of the gene in the absence of the candidate agent indicates that the candidate agent is an agent to be tested for the ability to prevent or inhibit addiction related behavior.

In some embodiments of the latter method, if at least one gene is detected the gene does not encode melanocortin 4 receptor.

In another embodiment, a method for identifying an agent to be tested for an ability to prevent or inhibit cocaine addiction-related behavior is provided which comprises:
a) contacting a brain tissue sample from each of a subject having a cocaine addiction-related behavior and a cocaine addiction-free subject;
b) detecting a level of expression of at least one gene in both tissue samples, wherein the gene encodes a polypeptide selected from the group consisting of hypertension-regulated vascular factor, myelin-associated basic protein, PB cadherin, calcitonin receptor, melanocortin 4 receptor, ALK-7 kinase and retroposon;
c) subtracting the level of expression of the gene in the sample obtained from the cocaine addiction-free subject from the level of expression of the gene in the sample obtained from the subject having cocaine addiction-related behavior to provide a first value;
d) administering a candidate agent to each of a subject having a cocaine addiction-related behavior and a cocaine addiction-free subject;
e) detecting a level of expression of the at least one gene in both tissue samples obtained from the subjects treated with the candidate agent;
f) subtracting the level of expression of the gene in the sample obtained from the treated cocaine addiction-free subject from the level of expression of the gene in the sample obtained from the treated subject having the cocaine addiction-related behavior to provide a second value; and
g) comparing the second value with the first value wherein a decreased second value relative to the first value is indicative of an agent useful in preventing or inhibiting the cocaine addiction-related behavior.

In yet another embodiment, a method for identifying an agent to be tested for an abiity to prevent or inhibit cocaine addiction-related behavior is provided which comprises:
a) obtaining a brain tissue sample from each of a subject having a cocaine addiction-related behavior and a cocaine addiction-free subject;
b) detecting a level of expression of at least one gene in both tissue samples, wherein the gene encodes a polypeptide selected from the group consisting of GABA-B receptor subunit gb2, cell adhesion-like molecule, bos taurus-like neuronal axonal protein, similar to mouse chemokine-like factor, FRA-2, similar to human oxygen-regulated protein, similar to mouse mrg1 protein, pentraxin, malic enzyme, olfactomedin-related protein, arc-growth factor enriched in dendrites, protein tyrosine phosphatase, krox, neuritin, microtubule-associated protein 2d and CB1 cannabinoid receptor;
c) subtracting the level of expression of the gene in the sample obtained from the cocaine addiction-free subject from the level of expression of the gene in the sample obtained from the subject having cocaine addiction-related behavior to provide a first value;
d) administering a candidate agent to each of a subject having a cocaine addiction-related behavior and a cocaine addiction-free subject;
e) detecting a level of expression of at least one gene in both tissue samples obtained from the subjects treated with the candidate agent;
f) subtracting the level of expression of the gene in the sample obtained from the treated cocaine addiction-free subject from the level of expression of the gene in the sample obtained from the treated subject having the cocaine addiction-related behavior to provide a second value; and
g) comparing the second value with the first value wherein an increased second value relative to the first value is indicative of an agent useful in preventing or inhibiting the cocaine addiction-related behavior.

The level of expression of at least one of the disclosed genes in the samples obtained from the subject and disease-free subject and brain cells obtained from the subjects can be detected by measuring either the level of mRNA corresponding to the gene or the protein encoded by the gene. RNA can be isolated from the samples by methods well-known to those skilled in the art as described e.g., in Ausubel et al., Current Protocols in Molecular Biology, Vol. 1, pp. 4.1.1-4.2.9 and 4.5.1-4.5.3, John Wiley & Sons, Inc. (1996).

Methods for detecting the level of expression of mRNA are well-known in the art and include, but are not limited to, Northern blotting, reverse transcription PCR, real time quantitative PCR and other hybridization methods.

A particularly useful method for detecting the level of mRNA transcripts obtained from a plurality of the disclosed genes involves hybridization of labeled mRNA to an ordered array of oligonucleotides. Such a method allows the level of transcription of a plurality of these genes, i.e., two or more, to be determined simultaneously to generate gene expression profiles or patterns. The gene expression profile derived from the sample obtained from the subject having the cocaine addiction-related behavior treated with agent can be compared with the gene expression profile derived from the sample obtained from the untreated subject having the cocaine addiction-related behavior to determine whether the genes are up- or down-regulated in the sample from the treated subject relative to the genes in the sample obtained from the untreated subject, and thereby determine whether the agent prevents or inhibits cocaine addition-related behavior.

The oligonucleotides utilized in this hybridization method are bound to a solid support. Examples of solid supports include, but are not limited to, membranes, filters, slides, paper, nylon, wafers, fibers, magnetic or nonmagnetic beads, gels, tubing, polymers, polyvinyl chloride dishes, etc. Any solid surface to which the oligonucletides can be bound, either directly or indirectly, either covalently or non-covalently, can be used. A particularly preferred solid substrate is a high-density array or DNA chip (see “Materials and Methods”; and Example 1). These high density arrays contain a particular oligonucleotide probe in a pre-selected location on the array. Each pre-selected location can contain more than one molecule of the particular probe. Because the oligonucleotides are at specified locations on the substrate, the hybridization patterns and intensities (which together result in a unique expression profile or pattern) can be interpreted in terms of expression levels of particular genes.

The oligonucleotide probes can be labeled with one or more labeling moieties to permit detection of the hybridized probe/target polynucleotide complexes. Label moieties can include compositions that can be detected by spectoscopic, biochemical, photochemical, bioelectronic, immunochemical, electrical optical or chemical means. Examples of labeling moieties include, but are not limited to, radioisotopes, e.g., ³²P, ³³P, ³⁵S, chemiluminescent compounds, labeled binding proteins, heavy metal atoms, spectoscopic markers such as fluorescent markers and dyes, linked enzymes, mass spectrometry tags and magnetic labels.

Oligonucleotide probe arrays for expression monitoring can be prepared and used according to techniques which are well-known to those skilled in the art as described, e.g., in Lockhart et al., Nat. Biotech., Vol. 14, pp. 1675-1680 (1996); McGall et al., Proc. Natl. Acad. Sci. USA, Vol. 93, pp. 13555-13460 (1996); and U.S. Pat. No. 6,040,138.

Expression of the protein encoded by the gene(s) can be detected by a probe which is detectably labeled, or which can be subsequently labeled. Generally, the probe is an antibody or other ligand which recognizes the expressed protein.

As used herein, the term “antibody” includes, but is not limited to, polyclonal antibodies, monoclonal antibodies, humanized or chimeric antibodies, and biologically functional antibody fragments which are those fragments sufficient for binding of the antibody fragment to the protein.

For the production of antibodies to a protein encoded by one of the disclosed genes, various host animals may be immunized by injection with the polypeptide, or a portion thereof. Such host animals may include, but are not limited to, rabbits, mice and rats, to name but a few. Various adjuvants may be used to increase the immunological response, depending on the host species, including but not limited to Freund's (complete and incomplete), mineral gels such as aluminum hydroxide, surface active substances, such as lysolecithin, pluronic polyols, polyanions, peptides, oil emulsions, keyhole limpet hemocyanin, dinitrophenol and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler et al., Nature, Vol. 256, pp. 495-497 (1975) and U.S. Pat. No. 4,376,110, the human B-cell hybridoma technique (see Kosbor et al., Immunology Today, Vol. 4, p. 72 (1983); Cole et al., Proc. Natl. Acad. Sci. USA, Vol. 80, pp. 2026-2030 (1983); and the EBV-hybridoma technique (see Cole et al., Monoclonal Antibodies and Cancer Therapy, Alan R. Liss, Inc., pp. 77-96 (1985)). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985)) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (see U.S. Pat. No. 4,946,778; Bird, Science, Vol. 242, pp. 423-426 (1988); Huston et al., Proc. Natl. Acad. Sci. USA, Vol. 85, pp. 5879-5883 (1988); and Ward et al., Nature, Vol. 334, pp. 544-546 (1989)) can be adapted to produce differentially expressed gene single-chain antibodies. Single-chain antibodies are formed by linking the heavy and light chain fragments of the Fv region via an amino acid bridge, resulting in a single-chain polypeptide.

Most preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments which recognize specific epitopes may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (see Huse et al., Science, Vol. 246, pp. 1275-1281 (1989)) to allow rapid and easy identification of monoclonal Fab fragments with the desired specificity.

The extent to which the known proteins are expressed in the sample is then determined by immunoassay methods which utilize the antibodies described above. Such immunoassay methods include, but are not limited to, dot blotting, Western blotting, competitive and non-competitive protein binding assays, enzyme-linked immunosorbant assays (ELISA), immunohistochemistry, fluorescence-activated cell sorting (FACS) and others commonly used and widely described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well-known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the protein expressed by the gene of interest.

The most commonly used reporter molecules in this type of assay are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of protein which is present in the serum sample. Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well-established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

The following examples are included to demonstrate preferred embodiments of the invention.

### EXAMPLES

Research Design and Methods

Strategy to Identify Changes in Gene Expression in the NAc Shell and other Brain Regions During Prolonged Abstinence

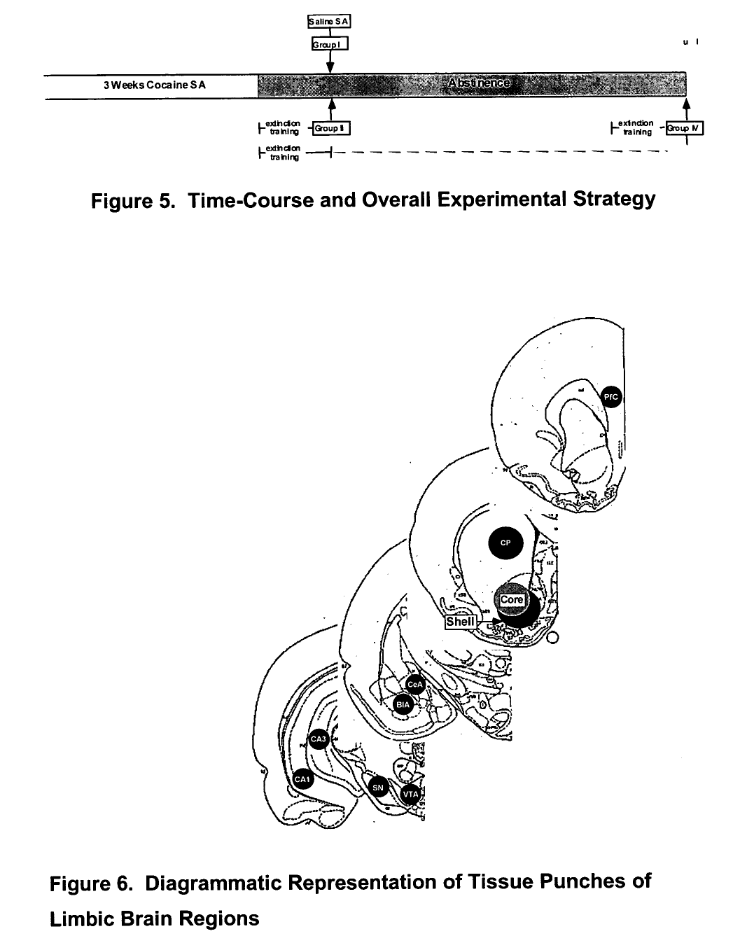

Six groups of rats (n=10/group) underwent 3 weeks (15 days) of daily (6-10 hours) cocaine self-administration, followed by short or long periods of forced abstinence prior to sacrifice. Changes in gene expression that coincide with time-dependent increases in cocaine-seeking behavior were identified by comparing changes in 1-week abstinence and 1-week extinction groups, as illustrated in FIG. 5 below (Groups I and II, respectively). First, a direct comparison between 1-week abstinence and 1-week extinction groups was conducted to identify differences. This allowed detection of genes that correspond to the groups with the greatest differences in drug-seeking behavior. Second, each experimental group (1 week abstinence and extinction) was directly compared to their respective untreated control groups to test whether differences between the groups represent reversals in gene expression between the withdrawal and extinction conditions. Direct comparisons with control groups also allowed detection of genes changed in withdrawal or extinction that might also contribute to drug-seeking behavior though their levels might not necessary be reversed between extinction and withdrawal.

Surgery, Behavioral Testing and Dissection of Specific Brain Regions

Both experimental and control groups consisted of individually housed, male Sprague Dawley rats. Experimental animals were surgically implanted with chronic, indwelling intravenous catheter as follows (see Sutton et al., supra). All surgery were performed under aseptic conditions, in a clean area used solely for surgical procedures. Each surgery was done on a separate, clean sheet of Whatman Benchkote paper. Surgical instruments were autoclaved and cleaned (cleaned and soaked in 70% ethanol between successive surgeries). Rats (at least 300 g) and mice (25-30 g) were anesthetized with an i.p. injection of pentobarbital (1.0 mg/kg; rats) and ketamine/xylazine (10 mL/kg; mice), and penicillin procaine intramuscular (i.m.) (60,000 IU/0.2 mL rats, 6,000 units/0.02 mL mice) was given as a prophylactic. The back area of the animals were shaved and cleaned with 70% ethanol, and 2 incisions were made, one on the back (2 cm), and one on the neck (1 cm). The jugular vein was isolated and a sterile Silastic catheter was inserted to the level sinus just outside the right atrium, and mounted in place with surgical mesh. The remaining catheter was pulled from the neck area subcutaneously back incision. Then the catheter exited via 22-gauge stainless steel tubing cemented into place with dental cement and skull screws on a plastic back mount. The incisions were sutured closed with silk surgical thread and the wounds treated with topical antibiotic, and the animal were given an i.m. injection of penicillin G procaine i.m. (60,000 IU/0.2 mL).

Rats implanted with intravenous (i.v.) catheter recovered from surgery on a warming pad. The rats were not used for experimentation for at least 4 days. During this time, each animal was monitored for distress or infection, and the catheter was flushed daily with 0.2 mL of heparinized saline (20 IU/mL/kg). Because prior exposure to analgesics can alter subsequent behavioral responses to drugs of abuse, rats did not receive post-operative analgesics. Controls remained in their home cages with frequent handling throughout the experiment. Experimental rats were allowed to self-administer cocaine by lever pressing (Fixed-ratio 1: Time-out 10 seconds, 0.5 mg/kg/injection) during their dark cycle 5 days/week for 3 weeks. Each cocaine infusion was delivered over 1.25 seconds concurrent with a cue light, and followed by a 10-second time-out period. The house-light was extinguished during the injection time-out period; together these stimuli constituted the cocaine cue used in reinstatement below. The experimental animals self-administered cocaine in contextually distinct operant chambers located in testing rooms outside the animal colony. During the first week, rats self-administered cocaine for 10 hours/day to hasten acquisition and accustom them to high levels of cocaine exposure. During the second and third weeks, animals self-administered cocaine 6 hours/day. These conditions typically produced self-regulated levels of cocaine intake of 50-60 mg/kg/6-hour test session at the end of self-administration testing, and more precisely mimic daily patterns of cocaine binges in humans.

Following 3 weeks of cocaine self-administration, animals were divided into experimental groups with equivalent mean levels of cocaine intake, and important factor that determines the propensity for cocaine-seeking during abstinence. Experimental Group II underwent extinction training for 5 days during the first week of abstinence for 6 hours/day, beginning 3 days after their final self-administration test session. Experimental Group IV underwent extinction training for 5 days during their sixth week of abstinence. Responding at both drug-paired and inactive lever were recorded during this time. On the last hour of the final extinction test session, cue-induced reinstatement of cocaine-seeking behavior was tested. During this hour, cues specifically associated with prior cocaine infusions during self-administration (house light off/cue light on) were presented every 2 minutes, and responding at the drug-paired and inactive levers were recorded. Experimental Group I remained in their home cages until sacrifice. Three more experimental groups underwent saline self-administration for 3 weeks and were sacrificed along with Groups I, II and IV. Each group consisted of 6-14 animals to reduce the effects of variability from individuals or dissection procedures on array profiling.

Animals undergoing extinction training were sacrificed 3 days after their last extinction training session; animals remaining in their home cages were sacrificed at similar times during abstinence. Animal were removed from their home cages and immediately sacrificed by decapitation. Brains were rapidly dissected and chilled slices in ice-cold artificial cerebral spinal fluid for 2 minutes. Tissue punches (12- to 16-gauge) were collected from serial coronal brain slices (1.2-1.5 mm thick) based on the locations depicted in FIG. 6. A 14-gauge punch was used to collect NAc core samples, and a 12-gauge punch was used to collect a “half moon” slice of the remaining NAc shell tissue, both yielding about 8-10 mg tissue/punch. Punches were rapidly frozen on dry ice, and stored at −80° C. until shipped to GNF for the GeneChip studies.

Isolation of Total RNA and Synthesis of cRNA Samples

Total RNA was isolated from pooled tissue samples using Trizol reagent (1 mL Trizol per 50 mg tissue) (Gibco BRL) and a homogenizer (Polytron, Kinematica) run at maximum speed for 90 seconds. The standard Trizol procedure was used, and RNA after ethanol precipitation was further purified with Rneasy columns (Qiagen). Quality of total RNA was assessed by agarose gel electrophoresis and quantity by spectrophotometer in water and Tris, pH 7.5. Yields were lower than expected and ranged from 4-20 μg. After gel electrophoresis and quantitation, the amount of the limiting sample was 3 μg. Due to the low yield, 250 nanogram aliquots were removed as a preventative measure in case cRNA yields were inadequate and a double amplification of the total RNA was needed. Complementary DNA (cDNA) was synthesized from 3 mg total RNA (corresponding to the amount of the sample with lowest yield) using a T7 promotor/oligo dT primer which allows for subsequent linear amplification of the resulting cDNA (see Van Gelder et al., “Amplified RNA Synthesized From Limited Quantities of Heterogeneous cDNA”, Proc. Natl. Acad. Sci. USA, Vol. 87, pp. 1663-1667 (1990)). This procedure results in cDNA and cRNA populations that accurately and reproducibly represent the total RNA of origin (see Lipshutz et al., “High Density Synthetic Oligonucleotide Arrays”, Nature Gen., Vol. 21, pp. 20-24 (1999); Lockhart et al., “Expression Monitoring by Hybridization to High-Density Oligonucleotide Arrays”, Nature Biotech., Vol. 14, pp. 1675-1680 (1996); and Wodicka et al., “Genome-Wide Expression Monitoring in Saccharomyces cerevisiae”, Nature Biotech., Vol. 15, pp. 1359-1367 (1997)). Briefly, 3 μg total RNA was used to make first strand cDNA using the Superscript Choice system (Gibco BRL) and a T7 promotor/oligodT primer (Gibco). Second strand cDNA was made with the Superscript Choice system. All of the resulting cDNA, after phenol:chloroform purification and ammonium acetate precipitation, was used as a template to make biotinylated amplified antisense cRNA using T7 RNA polymerase (Enzo kit, Affymetrix). Twenty micrograms cRNA was fragmented to a target range of 20-100 bases in length using fragmentation buffer (200 mM Tris-acetate, pH 8.1, 500 mM KOAc, 150 mM MgOAc) and heating for 35 minutes at 94° C. This procedure both reduces secondary structure of cRNA and prevents it from hybridizing to adjacent DNA probes on the array (Lockhart et al., supra and Southern et al., “Molecular Interactions on Microarrays”, Nature Gen., Vol. 21, pp. 5-9 (1999)). Quality of cRNA and size distribution of fragmented cRNA was examined by both agarose and polyacrylamide gel electrophoresis. It was determined that fragmentation did not yield the expected size range, and further fragmentation resulted in loss of sample. For this reason, the double amplification protocol was used.

Amplification and Labeling of Small Amounts of mRNA

Occasionally, yields of total RNA from small amounts of dissected brain regions is poor in quantity and yet of high quality. Thus, we used double linear amplification procedure as described (see Luo et al., “Gene Expression Profiles of Laser-Captured Adjacent Neuronal Subtypes”, [published erratum appears in Nat. Med., Vol. 5, No. 3, p. 355 (1999)] Nat. Med., Vol. 5, pp. 117-122 (1999)) and modified for use in our laboratory. First and second stranded cDNA was synthesized as described above using 50 ng starting total RNA, but first, unlabeled cRNA was made using the Megascript kit (Ambion). cRNA was purified with a microcon-50 column (Millipore) and cDNA was again made with random primers and Superscript II (GibcoBRL) at 37° C. for 1 hour, incubated at 37° C. in the presence of RNAse H (GibcoBRL) for 20 minutes. After heat denaturing the enzymes, a T7-oligo dT primer was added to the mixture and second strand cDNA was made with DNA polymerase I and then T4 DNA polymerase (GibcoBRL). cDNA was purified with microcon-50 columns (Millipore) and a second round of cRNA amplification was performed using the Enzo kit (Affymetrix). Unlike amplification by PCR, this method results in a linear amplification of the total RNA (above references). Between 39 and 84 μg of labeled cRNA was made from 50 ng starting total RNA. Twenty μg cRNA was fragmented as described above, fragmention was successful as determined by gel electrophoresis, and 15 μg fragmented cRNA was added to Affymetrix Gene Chip® Rat Genome U34 arrays with 1×MES hybridization buffer using standard protocols outlined in the Gene Chip® Expression Analysis Technical Manual (Affymetrix). Hybridization was for 16 hours at 45° C. The same hybridization samples were then removed from the chips and re-hybridized to identical arrays to make duplicates of each sample.

Washing, Staining and Scanning Arrays

Following hybridization of sample to arrays, sample was removed and arrays were washed to remove excess sample. Biotinylated cRNA that is specifically hybridized to the array was stained first with streptavidin phycoerythrin (SAPE, Molecular Probes), then with biotinylated anti-streptavidin antibody, and again with SAPE using standard protocols outlined in the Gene Chip® Expression Analysis Technical Manual (Affymetrix). Following washing, arrays were scanned with a laser scanner (Agilent). After scanning, Gene Chip® software aligns a grid to the image so that individual probe sets can be identified. The quantitative assessment of “present” or “absent” probe sets is based on the number of instances in which the PM signal is significantly larger than the MM signal across the redundant set of probes for each gene. This array design and analysis scheme is essentially a “voting” scheme. Determination of quantitative RNA abundance is made from the average of the pairwise differences (PM minus MM) across the set of probes for each RNA (average difference value). In order to compare average difference values for each RNA between different arrays, intensity values are scaled (normalized) using intensity values taken over the entire array. The Gene Chip® software makes qualitative calls of “Increase” or “Decrease” and quantitative assessments of the absolute size (“fold change”) of any differences. In order to increase confidence in the results, all experiments were performed using duplicate hybridizations. Only differences between duplicates are considered (see below).

Data Filtering to Find Differentially Expressed Genes (Primary Screen)

We have developed a Web-based software tool at our institute for gene expression array data filtering. This tool allows us to filter data with user-defined criteria. For example, if one is comparing gene expression changes between arrays A and B, fold changes are first made between A and B. Fold changes are also measured between duplicate arrays A′ and B′. Gene expression changes that are common between the duplicate comparisons are then selected. The criteria for valid differences are as follows:
Genes scored as “Increased”/“Moderately Increased” or “Decreased”/“Moderately Decreased” (by the standard Affymetrix algorithm) in both comparisons.
Genes with a minimum 2-fold change in both comparisons, and a minimum absolute change of 50 units in both comparisons.
Genes scored as “present” in the experimental file or “present” or “moderate” in the baseline file of at least one of the two comparisons.

This software tool can rapidly and accurately manage thousands of potentially regulated genes with a variety of filter settings. The stringency of the filter can be varied depending on the number of potentially regulated genes found. This same data filtering tool can also be used to examine the consistency of the duplicate arrays by finding the number of genes that are significantly “different” between duplicates.

A different data filtering approach was used to find differentially expressed genes in the NAc core, CeA, mPFC and VTA. The reasons for the change in the approach are that the new methods are easily adaptable to our gene expression database and they do not rely on “Increase, Decrease, Absence or Presence” calls generated by the Affymetrix algorithm. The Web-based tool used for finding gene expression changes in the NAc shell is less practical to use.

Two different filters were used to generate data for the NAc core, CeA, mPFC and VTA. The sum of the findings from both filters were used to generate the final gene lists, with redundant entries collapsed to generate one entry per probe set. The first filter used was a one-way ANOVA. Values less than a value of 20 were first forced to a value of 20, then ANOVA was performed.

Probe sets were retained in the gene lists only after they met the following criteria:
1. P-value less than 0.01.
2. Fold change difference between statistical groups at least 1.7.
3. Maximum intensity (average difference value) across the group of at least a value of 200.

The second filter used to generate data for the NAc core, CeA, mPFC and VTA avoided the potential problems of using ANOVA for small sample sizes. First, all values less than a value of 200 were forced to a value of 200. Then, mean values of the groups, standard deviations within the groups, and fold change differences between the groups were calculated and probe sets were retained only if they met the following criteria:
1. Fold change difference between groups at least 1.7.
2. The standard deviation of the group divided by the mean of the same group must have been a value of 0.25 or less for both groups.

Tissue Dissection/Western Blot Procedures

Rats were removed from their homecages and immediately decapitated in a separate room; the brains were rapidly dissected and chilled in ice-cold physiological buffer (5 mM KCl, 126 mM NaCl, 1.25 mM NaH₂PO₄, 10 mM D-glucose, 25 mM NaHCO₃, 2 mM CaCl₂, 2 mM MgSO₄, pH 7.4). NAc core samples were obtained with a 14-gauge punch from chilled coronal brain slices (0.7-2.2 mM anterior to bregma; Paxinos et al. (1998)), and immediately frozen and stored at −80° C. Half moon-shaped NAc shell samples were obtained with a 12-gauge punch of the remaining ventral-medial shell tissue.

Tissue samples were homogenized by sonication in 350 μL (NAc) of 1% SDS. Protein concentrations were determined (Lowry et al. (1951)), and 10 μg protein/sample was subjected to SDS-polyacrylamide gel electrophoresis (7.5-10% acrylamide/0.12% bisacrylamide), followed by electrophoretic transfer to nitrocellulose (Bio-Rad, Hercules, Calif.). Proteins were immunolabeled overnight at 4× in blocking buffer consisting of 5% non-fat dried milk powder in PBST (10 mM sodium phosphate, pH 7.4, 0.9% NaCl, 0.1% Tween-20). Following incubation with the primary antibody, blots were washed with blocking buffer, and incubated for 2 hours at 20° C. with horseradish peroxidase-conjugated goat anti-rabbit IgG (1:2000; Chemicon, Temecula, Calif.) in PBST. The blots were washed again in PBST, and immunoreactivity visualized using enhanced chemiluminescense for peroxidase labeling (New England Nuclear, Boston, Mass.). Protein immunoreactivity was quantified by densitometric analysis using NIH Image 1.57 (National Institute of Health, Bethesda, Md.). TH immunoreactivity was linear over a 4-fold range of tissue concentrations under these conditions.

Data Analysis

Each gel contained 7-11 control samples alternating with samples from experimental animals. To normalize data from different gels, protein immunoreactivity for each control and experimental sample was expressed as a percentage of the mean control value for that particular gel. For statistical analysis, age- and batch-matched control values were pooled into a single group, and compared with 2 cocaine-trained groups with 1-way ANOVA. Post-hoc comparisons were made among control and cocaine-trained groups with Newman Keuls tests.

Analysis of data from nucleus accumbens core, central nucleus of the amygdala, medial prefrontal cortex, and ventral tegmental area indicated that the 1 week withdrawal control and 1 week extinction control groups were not equivalent. Therefore pooling all of the control values into a single control group was not valid for these comparisons. Instead, extinction and withdrawal groups were compared directly or to their respective controls.

### Example 1

### Identification of Extinction/Withdrawal Differences in Gene Expression in the Nac Shell and Other Brain Regions During Prolonged Abstinence Using Gene Expression Profiling

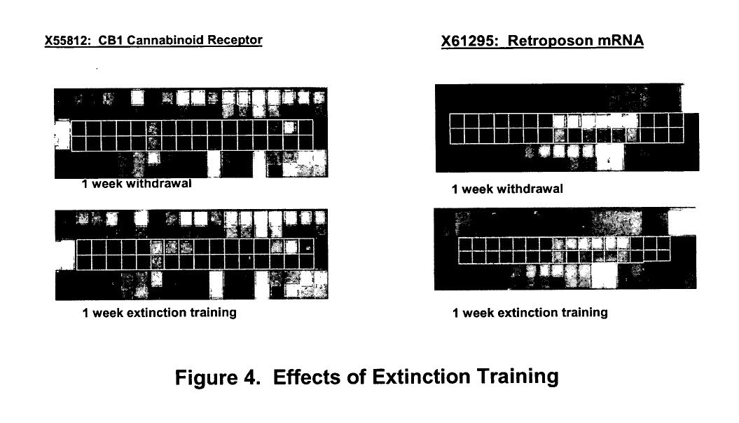

The advent of oligonucleotide arrays increases the feasibility of forward genetic approaches to identify gene regulation in studies of complex behaviors. This technology replaces more cumbersome methods of subtraction hybridization and differential display with the advantage of profiling thousands of genes simultaneously. FIG. 4 illustrates 2 candidate genes identified in our preliminary studies from contralateral NAc shell tissue samples taken from animals used in the extinction studies described above. These genes were selected by comparing 1-week extinction training and 1-week withdrawal groups according to stringent criteria described in the Research Design and Methods section. The top panel illustrates a 3.7-fold difference in expression of a retroviral derived gene retroposon (see Table 1). This gene is over-expressed in withdrawal from cocaine self-administration (88%), but down-regulated (49%) in animals that experienced extinction training when compared to untreated age- and batch-matched controls. In contrast, expression of the CB1 cannabinoid receptor gene is reduced (53%) in withdrawal, but normalized to near control levels following extinction training. Tables 1-15 contain all of the genes selected by both primary and secondary screening procedures for this comparison (see “Methods”). This procedure employs control/control comparisons to eliminate false positives, in addition to the gene filtering software-based selection procedure. As shown in Table 1, there are several genes for structural proteins (i.e., PB cadherin, microtubule-associated protein) suggesting neuroplasticity in neuronal contacts (dendritic spines and arborization). There are also 4 gene candidates (highlighted in bold) that already are implicated in drug reward and addiction. For example, GABA B receptor agonists have been proposed as a possible pharmacotherapy for cocaine addiction, and CB1 cannabinoid receptors mediate central effects of cannabis, and can modulate dopaminergic responses in striatum. Similarly, FRA2, is a Fos-Related Antigen like ΔFosB, which has been implicated in sensitivity to cocaine (see Kelz et al., “Expression of the Transcription Factor ΔFosB in the Brain Controls Sensitivity to Cocaine”, Nature, Vol. 401, pp. 272-276 (1999). The melanocortin receptor MC4 has recently been shown to be up-regulated during withdrawal from repeated cocaine treatments, and intra-NAc infusions of an MC4 antagonist reverse the rewarding effects of cocaine to produce a cocaine aversion instead in a place preference paradigm (see Taylor et al., “Role of Melanocortin in Drug Reward”, submitted).

TABLE 1
Effects of Extinction Training on Gene Expression in the NAc Shell
Following 1 Week Withdrawal from Cocaine Self-Administration

| | | | Extinction | Genbank |
|---|---|---|---|---|
| | 1 Week | 1 Week | vs. | Accession |
| Gene Name | Withdrawal* | Extinction* | Withdrawal | No. |
| GABA-B receptor subunit gb2 | ↓ 30% | ↑ 48% | 2.12-fold Δ | AJ011318.1 |
| Hypertension-regulated vascular factor | ↑ from 0 | 0 | Normalized | AF055714 |
| Myelin-associated basic protein | ↑ 140% | ↓ 7% | 2.59-fold Δ | X87900.1 |
| PB cadherin | ↑ 17% | ↓ 56% | 2.17-fold Δ | D83349.1 |
| Calcitonin receptor | ↑ 33% | ↓ 80% | 6.58-fold Δ | L13041.1 |
| Cell adhesion-like molecule | ↓ 88% | ↑ 6% | 8.92-fold Δ | M88709.1 |
| Bos taurus-like neuronal axonal protein | ↓ 36% | ↑ 34% | 2.08-fold Δ | U92535.1 |
| Similar to mouse chemokine-like factor | ↓ 47% | ↑ 65% | 2.08-fold Δ | AF144754.1 |
| FRA-2 | ↓ 66% | ↑ 41% | 3.21-fold Δ | X98051.1 |
| Similar to human oxygen regulated | ↓ 37% | ↑ 46% | 2.32-fold Δ | AI009098 |
| protein | | | | |
| Similar to mouse mrg1 protein | ↓ 48% | ↑ 87% | 3.62-fold Δ | AI014091 |
| Pentraxin | ↓ 70% | ↑ 63% | 5.41-fold Δ | U18772 |
| Malic enzyme | ↓ 33% | ↑ 61% | 2.39-fold Δ | M26594.1 |
| Olfactomedin related protein | ↓ 38% | ↑ 48% | 2.40-fold Δ | U03414 |
| Arc - growth factor enriched in | ↓ 45% | ↑ 21% | 2.18-fold Δ | U19866.1 |
| dendrites | | | | |
| Protein tyrosine phosphatase | ↓ 55% | ↑ 13% | 2.49-fold Δ | U28938 |
| Melanocortin 4 receptor | ↑ 272% | ↓ 35% | 4.21-fold Δ | U67863.1 |
| ALK-7 kinase | ↑ 44% | ↓ 44% | 2.57-fold Δ | U69702.1 |
| Krox | ↓ 47% | ↑ 15% | 2.19-fold Δ | U75397 |
| Neuritin | ↓ 87% | ↑ 28% | 10.1-fold Δ | U88958.1 |
| Microtubule-associated protein 2d | ↓ 17% | ↑ 67% | 2.02-fold Δ | X74211.1 |
| CB1 cannabinoid receptor | ↓ 53% | ↑ 19% | 2.52-fold Δ | X55812.1 |
| Retroposon | ↑ 88% | ↓ 49% | 3.70-fold Δ | U83119.1 |
| *Expressed as % Δ from mean control value for both groups (n = 5-8 pooled samples/group). Genes selected according to procedure described in Research Design and Methods. Gene names in bold indicate gene products in the NAc implicated in drug reward or addiction. | | | | |
| # Only changes in known genes are shown. Genes are selected based on criteria (see Methods) where both duplicate comparisons between extinction and withdrawal groups | | | | |
| # exceed 2-fold and are directionally similar. Base on this primary selection procedure, a secondary selection procedure eliminates genes when average duplicate values from both control groups vary more than 20% from the overall mean of the control groups. For genes expressed in | | | | |
| # low levels (<100 densitometric units), all control values must lie within 25 units of the overall mean. Average difference values for all groups and their respective control groups are shown in the Appendix tables. | | | | |

Thus, this latter neuroadaptation represents one difference replicated by alternative means (in situ). Several other genes regulated by withdrawal but not modified by extinction, and by extinction training alone are shown in Tables 2-16 below. These results demonstrate oligonucleotide detection of extinction/withdrawal differences.

TABLE 2
Average Difference Values for 1-Week Extinction Versus 1-Week Extinction Controls

| | | 1-Week | |
|---|---|---|---|
| | | Extinction | 1-Week |
| Probe Set | Gene Name | Control | Extinction |
| AF050659UTR#1_at | Activity and neurotransmitter-induced early 7 mRNA | 269 | 114 |
| AF050659UTR#1_at | | 347 | 132 |
| AJ000485_at | CLIP-115 protein | 95 | 168 |
| AJ000485_at | | 40 | 153 |
| AJ006971_g_at | DAP-like kinase | 184 | 545 |
| AJ006971_g_at | | 209 | 641 |
| D83348_at | Long-type PB cadherin | 113 | 285 |
| D83348_at | | 135 | 298 |
| K02248cds_s_at | Somatostatin-14 gene | 69 | 365 |
| K02248cds_s_at | | 132 | 460 |
| M13100cds#3_f_at | Long interspersed repetitive DNA sequence | 730 | 348 |
| M13100cds#3_f_at | | 938 | 474 |
| M16410_at | Neurokinin B precursor | 117 | 262 |
| M16410_at | | 110 | 241 |
| M32062_at | Fcgamma receptor | −19 | 96 |
| M32062_at | | 20 | 75 |
| M55015cds_s_at | Nucleolin gene | 49 | 154 |
| M55015cds_s_at | | 66 | 147 |
| M89646_g_at | Ribosomal protein S24 | 665 | 1466 |
| M89646_g_at | | 765 | 1370 |
| rc_AA799406_at | Genes for 18S, 5.8S and 28S ribosomal rRNAs | 244 | 683 |
| rc_AA799406_at | | −42 | 577 |
| rc_M800039_s_at | Unknown | 346 | 667 |
| rc_M800039_s_at | | 264 | 667 |
| rc_AA866419_at | Unknown | 59 | 150 |
| rc_AA866419_at | | −26 | 109 |
| rc_AA875268_at | Similar to B. taurus PSST subunit | 683 | 1332 |
| rc_AA875268_at | of NADH: ubiquinone oxidoreduc | 655 | 1361 |
| rc_AA891727_g_at | Unknown | 250 | 542 |
| rc_AA891727_g_at | | 285 | 576 |
| rc_AA891796_at | 1-cys peroxiredoxin; | 412 | 889 |
| rc_AA891796_at | thiol-specific antioxidant | 557 | 1180 |
| | protein | | |
| rc_AA892041_at | Homo sapiens over-expressed breast tumor | 768 | 1481 |
| rc_AA892041_at | protein mRNA | 788 | 1482 |
| rc_AA892123_at | Ribosomal protein L36 | 280 | 708 |
| rc_AA892123_at | | 378 | 761 |
| rc_AA892864_at | Unknown | 54 | 264 |
| rc_AA892864_at | | −2 | 259 |
| rc_AA924772_at | Growth inhibitory factor-metallothionein homolog | 1533 | 2979 |
| rc_AA924772_at | | 1577 | 3108 |
| rc_AI010581_at | 11 Kd diazepam binding inhibitor | 249 | 569 |
| rc_AI010581_at | | 246 | 570 |
| rc_AI014135_g_at | CDK103 | 822 | 340 |
| rc_AI014135_g_at | | 916 | 317 |
| rc_AI171844_at | F1-aTPase epsilon subunit | 563 | 1232 |
| rc_AI171844_at | | 564 | 1345 |
| rc_AI176460_s_at | 32S pre-rRNA 5′ terminal part with 28S rRNA sequence | 1640 | 3545 |
| rc_AI176460_s_at | | 1728 | 3573 |
| rc_AI227887_at | Similar to Mus musculus CDC42 mRNA | 304 | 7 |
| rc_AI227887_at | | 334 | 119 |
| rc_AI639367_at | Unknown | 574 | 63 |
| rc_AI639367_at | | 605 | 81 |
| rc_AI639521_at | Unknown | 141 | 2 |
| rc_AI639521_at | | 109 | 21 |
| U75392_s_at | B-cell receptor associated protein 37 | 191 | 516 |
| U75392_s_at | | 190 | 488 |
| X02002_at | Thy-1 gene for cell surface glycoprotein | 197 | 482 |
| X02002_at | | 251 | 498 |
| X05472cds#1_s_at | 2.4 Kb repeat DNA right terminal region | 428 | 169 |
| X05472cds#1_s_at | | 311 | 123 |
| X14671cds_s_at | Liver mRNA for ribosomal protein L26 | 871 | 1811 |
| X14671cds_s_at | | 1050 | 1949 |
| X53581cds#5_f_at | Long interspersed repetitive DNA sequence | 460 | 125 |
| X53581cds#5_f_at | | 425 | 109 |
| X55153mRNA_s_at | RP2 gene for ribosomal protein P2 | 723 | 1603 |
| X55153mRNA_s_at | | 596 | 1639 |
| X56325mRNA_s_at | Alpha-1 globin gene | 1886 | 3834 |
| X56325mRNA_s_at | | 1848 | 4115 |
| X61295cds_s_at | L1 retroposon mRNA | 1299 | 635 |
| X61295cds_s_at | | 1080 | 529 |
| X62952_at | Vimentin | −60 | 117 |
| X62952_at | | 26 | 117 |
| X63594cds_g_at | RL/IF-1 | −32 | 121 |
| X63594cds_g_at | | 48 | 194 |
| X68283_at | Ribosomal protein L29 | 703 | 1462 |
| X68283_at | | 524 | 1271 |
| Y13714_at | Osteonectin | 174 | 531 |
| Y13714_at | | 187 | 505 |

Genes that passed the filtering criteria outlined above for the nucleus accumbens shell are listed. Average difference values (from GeneChip version 3.2) are listed for each gene from each duplicate chip from both the 1 week extinction and 1 week extinction control groups. Affymetrix probe set numbers are listed along with the common name of the genes, if known.

TABLE 3
Average Difference Values for 1-Week Extinction, 1-Week
Withdrawal and Their Corresponding Control Groups

| | | | | 1-week | 1-week |
|---|---|---|---|---|---|
| | | 1-week | 1-week | extinction | withdrawal |
| Probe set no. | Gene name | extinction | withdrawal | control | control |
| AF055714UTR#1_at | Hypertension-regulated | −14 | 63 | −22 | −22 |
| AF055714UTR#1_at | vascular factor | −14 | 55 | −23 | −3 |
| AF058795_at | GABA-B receptor subunit gb2 | 621 | 309 | 432 | 468 |
| AF058795_at | | 695 | 311 | 456 | 421 |
| D28111_at | Myelin-associated basic | 995 | 2551 | 879 | 1037 |
| D28111_at | protein | 835 | 2186 | 783 | 1243 |
| D83349_at | PB cadherin | 1709 | 3903 | 2960 | 3704 |
| D83349_at | | 1798 | 3703 | 2580 | 3759 |
| L13040_s_at | Calcitonin receptor | 11 | 149 | 124 | 83 |
| L13040_s_at | | 32 | 134 | 129 | 91 |
| M13100cds#1_at | Long repetitive sequence | 895 | 2774 | 237 | 1335 |
| M13100cds#1_at | | 910 | 3278 | 414 | 1220 |
| M13100cds#1_g_at | Long repetitive sequence | 117 | 297 | 1564 | 270 |
| M13100cds#1_g_at | | 84 | 293 | 1181 | 375 |
| M13100cds#2_s_at | Long repetitive sequence | 177 | 892 | 195 | 408 |
| M13100cds#2_s_at | | 181 | 781 | 174 | 355 |
| M13100cds#3_f_at | Long repetitive sequence | 348 | 926 | 547 | 1001 |
| M13100cds#3_f_at | | 474 | 1206 | 483 | 974 |
| M13100cds#4_f_at | Long repetitive sequence | 153 | 547 | 730 | 229 |
| M13100cds#4_f_at | | 89 | 420 | 938 | 192 |
| M13100cds#5_s_at | Long repetitive sequence | 212 | 802 | 249 | 390 |
| M13100cds#5_s_at | | 157 | 765 | 175 | 342 |
| M13100cds#6_f_at | Long repetitive sequence | 273 | 916 | 425 | 766 |
| M13100cds#6_f_at | | 185 | 626 | 384 | 771 |
| M13101cds_f_at | Unknown | 57 | 371 | 746 | 307 |
| M13101cds_f_at | | 153 | 588 | 612 | 439 |
| M88709_at | Cell adhesion-like molecule | 341 | 65 | 262 | 264 |
| M88709_at | | 230 | −1 | 212 | 339 |
| rc_AA799423_at | Unknown | 79 | 292 | 172 | 168 |
| rc_AA799423_at | | 71 | 201 | 193 | 244 |
| rc_AA799448_g_at | Unknown | 470 | 90 | 392 | 385 |
| rc_AA799448_g_at | | 462 | 218 | 489 | 347 |
| rc_AA799594_at | Unknown | 1974 | 3970 | 1692 | 1572 |
| rc_AA799594_at | | 1497 | 3251 | 2238 | 2138 |
| rc_AA859536_at | Similar to Bos taurus neuronal | 3524 | 1672 | 2666 | 2273 |
| rc_AA859536_at | axonal membrane protein | 3517 | 1707 | 2885 | 2675 |
| rc_AA874803_g_at | Similar to mouse chemokine- | 1515 | 488 | 896 | 802 |
| rc_AA874803_g_at | like factor | 1485 | 479 | 1023 | 907 |
| rc_AA875001_at | Unknown | 255 | −72 | 213 | 221 |
| rc_AA875001_at | | 270 | 24 | 224 | 278 |
| rc_AA875032_at | FRA-2 | 285 | 102 | 193 | 222 |
| rc_AA875032_at | | 344 | 94 | 263 | 214 |
| rc_AI009098_at | Highly similar to human | 612 | 292 | 491 | 390 |
| rc_AI009098_at | oxygen-regulated protein | 537 | 204 | 344 | 346 |
| rc_AI014091_at | Highly similar to mouse mrg1 | 231 | 84 | 36 | 195 |
| rc_AI014091_at | protein (a cytokine-inducible | 269 | 54 | 138 | 165 |
| | transcr. | | | | |
| rc_AI014135_g_at | CDK103 | 340 | −20 | 822 | 332 |
| rc_AI014135_g_at | | 317 | 27 | 916 | 368 |
| rc_AI072943_at | Pentraxin | 167 | 48 | 68 | 51 |
| rc_AI072943_at | | 55 | −7 | 51 | 103 |
| rc_AI073204_at | 14-33 protein epsilon | 1793 | 561 | 1398 | 440 |
| rc_AI073204_at | | 1535 | 587 | 1340 | 448 |
| rc_AI171506_at | Malic enzyme | 95 | 28 | 82 | 79 |
| rc_AI171506_at | | 118 | 61 | 54 | 50 |
| rc_AI176710_at | Nuclear orphan receptor | 358 | 62 | 144 | 272 |
| rc_AI176710_at | | 305 | 54 | 163 | 252 |
| rc_AI231445_at | Lysosomal glycoprotein | −80 | 17 | 2 | 17 |
| rc_AI231445_at | | −12 | 39 | 31 | 4 |
| rc_AI233362_at | Unknown | 919 | 2280 | 1405 | 1088 |
| rc_AI233362_at | | 1045 | 2321 | 1359 | 1073 |
| rc_AI639088_s_at | Unknown | 116 | 377 | 353 | 290 |
| rc_AI639088_s_at | | 92 | 350 | 267 | 251 |
| rc_AI639118_at | Unknown | 143 | 70 | 98 | 119 |
| rc_AI639118_at | | 130 | 43 | 94 | 128 |
| rc_AI639226_at | Unknown | 28 | 91 | 65 | 81 |
| rc_AI639226_at | | 17 | 73 | 90 | 80 |
| rc_AI639367_at | Unknown | 63 | 530 | 574 | 553 |
| rc_AI639367_at | | 81 | 453 | 605 | 405 |
| rc_AI639484_at | Unknown | 1520 | 509 | 1243 | 1265 |
| rc_AI639484_at | | 1539 | 612 | 1194 | 1385 |
| rc_AI639521_at | Alpha beta crystalline gene | 2 | 99 | 141 | 103 |
| rc_AI639521_at | | 21 | 84 | 109 | 141 |
| rc_H31118_at | Unknown | 1247 | 430 | 1152 | 810 |
| rc_H31118_at | | 1227 | 492 | 1176 | 842 |
| U03414_s_at | Olfactomedin-related protein | 1183 | 534 | 797 | 785 |
| U03414_s_at | | 1194 | 458 | 907 | 731 |
| U03416_at | Olfactomedin-related protein | 1184 | 471 | 803 | 737 |
| U03416_at | | 1186 | 508 | 846 | 859 |
| U19866_at | Arc - a growth factor enriched | 815 | 403 | 683 | 594 |
| U19866_at | in dendrites | 627 | 257 | 549 | 557 |
| U28938_at | Protein tyrosine phosphatase | 461 | 184 | 320 | 416 |
| U28938_at | | 440 | 178 | 426 | 435 |
| U67863_at | Melanocortin 4 receptor | 14 | 125 | 21 | 38 |
| U67863_at | | 39 | 98 | 56 | 49 |
| U69702_at | ALK-7 kinase | 67 | 188 | 140 | 128 |
| U69702_at | | 80 | 190 | 125 | 132 |
| U75397UTR#1_s_at | Krox | 1077 | 461 | 964 | 983 |
| U75397UTR#1_s_at | | 1010 | 494 | 887 | 793 |
| U83119_f_at | Repetitive DNA sequence | 68 | 393 | 314 | 730 |
| U83119_f_at | | 38 | 426 | 379 | 484 |
| U88958_at | Neuritin | 260 | 40 | 244 | 216 |
| U88958_at | | 257 | 11 | 158 | 192 |
| U95920_at | Precentriolar material | 107 | 233 | 157 | 161 |
| U95920_at | | 102 | 200 | −32 | 129 |
| X01118_at | Atrial natriuretic polypeptie | 109 | −15 | −34 | 40 |
| X01118_at | | 124 | −17 | 10 | 12 |
| X05472cds#1_s_at | Repeat DNA | 169 | 624 | 428 | 422 |
| X05472cds#1_s_at | | 123 | 633 | 311 | 317 |
| X05472cds#2_at | Repeat DNA | 660 | 1396 | 931 | 630 |
| X05472cds#2_at | | 630 | 1412 | 807 | 627 |
| X05472cds#3_f_at | Repeat DNA | 133 | 968 | 213 | 188 |
| X05472cds#3_f_at | | 100 | 878 | 210 | 195 |
| X07686cds_s_at | Repeat DNA | 58 | 291 | 121 | 135 |
| X07686cds_s_at | | 28 | 275 | 112 | 112 |
| X17682_s_at | Microtubule-associated | 649 | 319 | 352 | 414 |
| X17682_s_at | protein | 596 | 298 | 335 | 388 |
| X53455cds_s_at | Microtubule-associated | 225 | 33 | 126 | 217 |
| X53455cds_s_at | protein | 299 | 76 | 53 | 161 |
| X53581cds#5_f_at | Repeat DNA | 125 | 366 | 460 | 411 |
| X53581cds#5_f_at | | 109 | 471 | 425 | 768 |
| X55812complete_seq_at | CB1 Cannabinoid receptor | 294 | 99 | 208 | 251 |
| X55812complete_seq_at | | 268 | 124 | 247 | 240 |
| X61295cds_s_at | Retroposon | 635 | 2177 | 1299 | 1181 |
| X61295cds_s_at | | 529 | 2128 | 1080 | 1022 |

Genes that passed the filtering criteria outlined above for the nucleus accumbens shell are listed. Average difference values (from GeneChip version 3.2) are listed for each gene from each duplicate chip from all groups. Affymetrix probe set numbers are listed along with the common name of the genes, if known.

TABLE 4
CeA 1-Week Extinction to Control

| | | | | | 1-week | | | | |
|---|---|---|---|---|---|---|---|---|---|
| | | | 1-week | 1-week | with- | | | | |
| | | 1-Week | with- | extinction | drawal | Mean | Mean | | Fold |
| Probe set no. | Description | extinction | drawal | control | control | control | extinction | Ratio | change |
| AB016161cds_i_at | AB016161cds Rattus | 352 | 460 | 50 | 234 | 406 | 142 | 0.349754 | −2.9 |
| | norvegicus mRNA for | | | | | | | | |
| | GABAB receptor 1d, | | | | | | | | |
| | complete cds | | | | | | | | |
| AF010466_s_at | AF010466 Rattus | 13 | −24 | 298 | 411 | −5.5 | 354.5 | −64.4545 | at least |
| | norvegicus interferon | | | | | | | | 2-fold |
| | gamma (IFN-gamma) | | | | | | | | |
| | mRNA, complete cds | | | | | | | | |
| AF031430_at | AF031430 Rattus | 227 | 229 | 103 | 119 | 228 | 111 | 0.486842 | −2.1 |
| | norvegicus syntaxin 7 | | | | | | | | |
| | mRNA, complete cds | | | | | | | | |
| AF042830_at | AF042830 Rattus | 433 | 361 | 253 | 206 | 397 | 229.5 | 0.578086 | −1.7 |
| | norvegicus proto-oncogene | | | | | | | | |
| | tyrosine kinase receptor | | | | | | | | |
| | Ret (c-ret) mRNA, partial | | | | | | | | |
| | cds | | | | | | | | |
| AF102552_s_at | AF102552 Rattus | 416 | 499 | 216 | 238 | 457.5 | 227 | 0.496175 | −2.0 |
| | norvegicus 270 kDa | | | | | | | | |
| | ankyrin G isoform mRNA, | | | | | | | | |
| | partial cds | | | | | | | | |
| D13962_g_at | D13962 RATGLUT3 Rat | 358 | 341 | 177 | 149 | 349.5 | 163 | 0.466381 | −2.1 |
| | mRNA for neuron glucose | | | | | | | | |
| | transporter | | | | | | | | |
| D17711cds_s_at | D17711cds RATCSBP Rat | 301 | 296 | 159 | 149 | 298.5 | 154 | 0.515913 | −1.9 |
| | mRNA for dC-stretch | | | | | | | | |
| | binding protein (CSBP), | | | | | | | | |
| | complete cds | | | | | | | | |
| D21800_g_at | D21800 RATPSRC10 Rat | 110 | 114 | 269 | 252 | 112 | 260.5 | 2.325893 | 2.3 |
| | mRNA for proteasome | | | | | | | | |
| | subunit RC10-II, complete | | | | | | | | |
| | cds | | | | | | | | |
| D26154UTR#1_at | D26154UTR#1 RATRB109 | 532 | 427 | 286 | 220 | 479.5 | 253 | 0.527633 | −1.9 |
| | Rat mRNA for RB109 | | | | | | | | |
| | (brain specific protein), | | | | | | | | |
| | complete cds | | | | | | | | |
| D26500_at | D26500 RATDLP9A Rat | 277 | 271 | 137 | 161 | 274 | 149 | 0.543796 | −1.8 |
| | mRNA for dynein-like | | | | | | | | |
| | protein 9A, partial cds | | | | | | | | |
| D82071_at | D82071 Rattus norvegicus | 207 | 196 | 94 | 81 | 201.5 | 87.5 | 0.434243 | −2.3 |
| | mRNA for hematopoietic | | | | | | | | |
| | prostaglandin D synthase, | | | | | | | | |
| | complete cds/cds = 192,791/ | | | | | | | | |
| | gb = D82071/gi = 2558504/ | | | | | | | | |
| | ug = Rn.10837/len = 1004 | | | | | | | | |
| E13644cds_s_at | E13644cds cDNA | 313 | 292 | 151 | 165 | 302.5 | 158 | 0.522314 | −1.9 |
| | encoding Neurodap-1 | | | | | | | | |
| | which is located at the | | | | | | | | |
| | post-synaptic membrane | | | | | | | | |
| | thickening regions of | | | | | | | | |
| | neurons and contains | | | | | | | | |
| | RING-H2 finger motif | | | | | | | | |
| J00771_at | J00771 RATPRNASE Rat | 173 | 139 | 430 | 353 | 156 | 391.5 | 2.509615 | 2.5 |
| | pancreatic ribonuclease | | | | | | | | |
| | mRNA | | | | | | | | |
| L07398_at | L07398 RATIGVCL Rattus | 670 | 687 | 290 | 280 | 678.5 | 285 | 0.420044 | −2.4 |
| | norvegicus (hybridoma | | | | | | | | |
| | 56R-3) immunoglobulin | | | | | | | | |
| | rearranged gamma-chain | | | | | | | | |
| | mRNA variable (V) region, | | | | | | | | |
| | partial cds | | | | | | | | |
| M12112mRNA#3_s_at | M12112mRNA#3 | 347 | 244 | 502 | 657 | 295.5 | 579.5 | 1.961083 | 2.0 |
| | RATANGA2 Rat | | | | | | | | |
| | angiotensinogen mRNA, | | | | | | | | |
| | 3′ flank | | | | | | | | |
| M34331_at | M34331 Rat 60S ribosomal | 733 | 704 | 1155 | 1508 | 718.5 | 1331.5 | 1.853166 | 1.9 |
| | subunit protein L35 mRNA, | | | | | | | | |
| | complete cds/cds = 47,418/ | | | | | | | | |
| | gb = M34331/gi = 206729/ | | | | | | | | |
| | ug = Rn.3458/len = 451 | | | | | | | | |
| rc_AI639304_at | Rat mixed-tissue library | 542 | 524 | 301 | 325 | 533 | 313 | 0.587242 | −1.7 |
| | Rattus norvegicus cDNA | | | | | | | | |
| | clone rx00157 3′, mRNA | | | | | | | | |
| | sequence [Rattus | | | | | | | | |
| | norvegicus] | | | | | | | | |
| rc_AA799489_g_at | rc_AA799489 EST188986 | 108 | −84 | 373 | 483 | 12 | 428 | 35.66667 | 35.7 |
| | Rattus norvegicus cDNA, | | | | | | | | |
| | 3′ end/clone = RHEAB66/ | | | | | | | | |
| | clone_end = 3′/ | | | | | | | | |
| | gb = AA799489/gi = 2862444/ | | | | | | | | |
| | ug = Rn.6193/len = 646 | | | | | | | | |
| rc_AA799498_at | rc_AA799498 EST188995 | 375 | 495 | 44 | 47 | 435 | 45.5 | 0.104598 | −9.6 |
| | Rattus norvegicus cDNA, | | | | | | | | |
| | 3′ end/clone = RHEAB76/ | | | | | | | | |
| | clone_end = 3′/ | | | | | | | | |
| | gb = AA799498/gi = 2862453/ | | | | | | | | |
| | ug = Rn.3835/len = 683 | | | | | | | | |
| rc_AA800549_at | rc_AA800549 EST190046 | 275 | 316 | 461 | 655 | 295.5 | 558 | 1.888325 | 1.9 |
| | Rattus norvegicus cDNA, | | | | | | | | |
| | 3′ end/clone = RLUAB29/ | | | | | | | | |
| | clone_end = 3′/ | | | | | | | | |
| | gb = AA800549/gi = 2863504/ | | | | | | | | |
| | ug = Rn.22957/len = 491 | | | | | | | | |
| rc_AA800882_g_at | rc_AA800882 EST190379 | 204 | 166 | 436 | 403 | 185 | 419.5 | 2.267568 | 2.3 |
| | Rattus norvegicus cDNA, | | | | | | | | |
| | 3′ end/clone = RLUAM60/ | | | | | | | | |
| | clone_end = 3′/ | | | | | | | | |
| | gb = AA800882/gi = 2863837/ | | | | | | | | |
| | ug = Rn.24136/len = 379 | | | | | | | | |
| rc_AA818114_at | rc_AA818114 UI-R-A0-am- | 210 | 227 | 107 | 101 | 218.5 | 104 | 0.475973 | −2.1 |
| | g-03-0-UI.s1 Rattus | | | | | | | | |
| | norvegicus cDNA, 3′ end/ | | | | | | | | |
| | clone = UI-R-A0-am-g-03- | | | | | | | | |
| | 0-UI/clone_end = 3′/ | | | | | | | | |
| | gb = AA818114/gi = 2887994/ | | | | | | | | |
| | ug = Rn.7181/len = 556 | | | | | | | | |
| rc_AA851403_at | rc_AA851403 EST194171 | 474 | 453 | 296 | 209 | 463.5 | 252.5 | 0.544768 | −1.8 |
| | Rattus norvegicus cDNA, | | | | | | | | |
| | 3′ end/clone = RPLAG17/ | | | | | | | | |
| | clone_end = 3′/ | | | | | | | | |
| | gb = AA851403/gi = 2938943/ | | | | | | | | |
| | ug = Rn.3383/len = 393 | | | | | | | | |
| rc_AA859643_at | rc_AA859643 UI-R-E0-bs- | 597 | 468 | 243 | 137 | 532.5 | 190 | 0.356808 | −2.8 |
| | a-08-0-UI.s1 Rattus | | | | | | | | |
| | norvegicus cDNA, 3′ end/ | | | | | | | | |
| | clone = UI-R-E0-bs-a-08-0- | | | | | | | | |
| | UI/clone_end = 3′/ | | | | | | | | |
| | gb = AA859643/gi = 2949163/ | | | | | | | | |
| | ug = Rn.32/len = 482 | | | | | | | | |
| rc_AA875659_s_at | rc_AA875659 UI-R-E0-ct- | 157 | 285 | 390 | 485 | 221 | 437.5 | 1.979638 | 2.0 |
| | h-07-0-UI.s1 Rattus | | | | | | | | |
| | norvegicus cDNA, 3′ end/ | | | | | | | | |
| | clone = UI-R-E0-ct-h-07-0- | | | | | | | | |
| | UI/clone_end = 3′/ | | | | | | | | |
| | gb = AA875659/gi = 2980607/ | | | | | | | | |
| | ug = Rn.10966/len = 424 | | | | | | | | |
| rc_AA891222_at | rc_AA891222 EST195025 | 380 | 322 | 150 | 98 | 351 | 124 | 0.353276 | −2.8 |
| | Rattus norvegicus cDNA, | | | | | | | | |
| | 3′ end/clone = RHEAQ71/ | | | | | | | | |
| | clone_end = 3′/ | | | | | | | | |
| | gb = AA891222/gi = 3018101/ | | | | | | | | |
| | ug = Rn.1014/len = 568 | | | | | | | | |
| rc_AA891940_at | rc_AA891940 EST195743 | 52 | 212 | 385 | 426 | 132 | 405.5 | 3.07197 | 3.1 |
| | Rattus norvegicus cDNA, | | | | | | | | |
| | 3′ end/clone = RKIAI82/ | | | | | | | | |
| | clone_end = 3′/ | | | | | | | | |
| | gb = AA891940/gi = 3018819/ | | | | | | | | |
| | ug = Rn.3508/len = 523 | | | | | | | | |
| rc_AA894292_at | rc_AA894292 EST198095 | 441 | 319 | 215 | 222 | 380 | 218.5 | 0.575 | −1.7 |
| | Rattus norvegicus cDNA, | | | | | | | | |
| | 3′ end/clone = RSPAW06/ | | | | | | | | |
| | clone_end = 3′/ | | | | | | | | |
| | gb = AA894292/gi = 3021171/ | | | | | | | | |
| | ug = Rn.19450/len = 599 | | | | | | | | |
| rc_AA924772_at | rc_AA924772 UI-R-A1-eb- | 98 | 266 | 499 | 691 | 182 | 595 | 3.269231 | 3.3 |
| | f-02-0-UI.s1 Rattus | | | | | | | | |
| | norvegicus cDNA, 3′ end/ | | | | | | | | |
| | clone = UI-R-A1-eb-f-02-0- | | | | | | | | |
| | UI/clone_end = 3′/ | | | | | | | | |
| | gb = AA924772/gi = 3071908/ | | | | | | | | |
| | ug = Rn.11325/len = 372 | | | | | | | | |
| rc_AI070108_at | rc_AI070108 UI-R-Y0-Iu-a- | 377 | 336 | 164 | 121 | 356.5 | 142.5 | 0.399719 | −2.5 |
| | 09-0-UI.s1 Rattus | | | | | | | | |
| | norvegicus cDNA, 3′ end/ | | | | | | | | |
| | clone = UI-R-Y0-Iu-a-09-0- | | | | | | | | |
| | UI/clone_end = 3′/ | | | | | | | | |
| | gb = AI070108/ug = Rn.16863/ | | | | | | | | |
| | len = 529 | | | | | | | | |
| rc_AI137421_at | rc_AI137421 UI-R-C2p-ok- | 163 | 193 | 442 | 479 | 178 | 460.5 | 2.587079 | 2.6 |
| | c-12-0-UI.s1 Rattus | | | | | | | | |
| | norvegicus cDNA, 3′ end/ | | | | | | | | |
| | clone = UI-R-C2p-ok-c-12-0- | | | | | | | | |
| | UI/clone_end = 3′/ | | | | | | | | |
| | gb = AI137421/ug = Rn.1485/ | | | | | | | | |
| | len = 556 | | | | | | | | |
| U04934_s_at | U04934 RNU04934 Rattus | 435 | 421 | 147 | 284 | 428 | 215.5 | 0.503505 | −2.0 |
| | norvegicus Sprague- | | | | | | | | |
| | Dawley (CD-1) clone Kc1 | | | | | | | | |
| | Na-Ca exchanger mRNA, | | | | | | | | |
| | partial cds | | | | | | | | |
| U75899mRNA_g_at | U75899mRNA RNU75899 | 791 | 664 | 378 | 462 | 727.5 | 420 | 0.57732 | −1.7 |
| | Rattus norvegicus HSPB2 | | | | | | | | |
| | gene, complete cds | | | | | | | | |
| X58830_at | X58830 Rat vgr mRNA/ | 503 | 484 | 275 | 277 | 493.5 | 276 | 0.559271 | −1.8 |
| | cds = 0.623/gb = X58830/ | | | | | | | | |
| | gi = 57475/ug = Rn.10436/ | | | | | | | | |
| | len = 1241 | | | | | | | | |
| Z50052_at | Z50052 R. norvegicus | 214 | 232 | 71 | 69 | 223 | 70 | 0.313901 | −3.2 |
| | mRNA for C4BP beta chain | | | | | | | | |
| | protein/cds = 265.1041/ | | | | | | | | |
| | gb = Z50052/gi = 899381/ | | | | | | | | |
| | ug = Rn.11151/len = 1091 | | | | | | | | |

Genes that passed the filtering criteria outlined above for differential expression between 1 week extinction and its corresponding control group in the CeA. Average difference values (from GeneChip version 3.2) are listed for each gene from all groups. Affymetrix probe set numbers are listed along with the common name of the genes, if known.

TABLE 5
CeA 1-Week Extinction to Withdrawal

| | | | | | 1-week | | | | |
|---|---|---|---|---|---|---|---|---|---|
| | | | 1-week | 1-week | with- | | | | |
| | | 1-week | with- | extinction | drawal | Mean | Mean | | Fold |
| Probe set no. | Description | extinction | drawal | control | control | control | extinction | Ratio | change |
| AB016161cds_i_at | AB016161cds Rattus | 352 | 460 | 50 | 234 | 406 | 142 | 0.349754 | −2.9 |
| | norvegicus mRNA for | | | | | | | | |
| | GABAB receptor 1d, | | | | | | | | |
| | complete cds | | | | | | | | |
| AB000517_s_at | AB000517 Rattus sp. | 300 | 387 | 162 | 146 | 343.5 | 154 | 0.448326 | −2.2 |
| | mRNA for CDP- | | | | | | | | |
| | diacylglycerol synthase, | | | | | | | | |
| | complete cds | | | | | | | | |
| AF015304_at | AF015304 Rattus | 434 | 419 | 201 | 200 | 426.5 | 200.5 | 0.470106 | −2.1 |
| | norvegicus equilbrative | | | | | | | | |
| | nitrobenzylthioinosine- | | | | | | | | |
| | sensitive nucleoside | | | | | | | | |
| | transporter mRNA, | | | | | | | | |
| | complete cds/cds = 4.1377/ | | | | | | | | |
| | gb = AF015304/gi = 2656136/ | | | | | | | | |
| | ug = Rn.5814/len = 1766 | | | | | | | | |
| AF041373_s_at | AF041373 Rattus | 468 | 405 | 190 | 8 | 436.5 | 99 | 0.226804 | −4.4 |
| | norvegicus clathrin | | | | | | | | |
| | assembly protein short form | | | | | | | | |
| | (CALM) mRNA, complete | | | | | | | | |
| | cds/cds = 25.1818/ | | | | | | | | |
| | gb = AF041373/gi = 2792499/ | | | | | | | | |
| | ug = Rn.10888/len = 1921 | | | | | | | | |
| AF064856_at | AF064856 Rattus sp. | 332 | 244 | 514 | 540 | 288 | 527 | 1.829861 | 1.8 |
| | 7acomp protein mRNA, | | | | | | | | |
| | complete cds | | | | | | | | |
| E00775cds_s_at | E00775cds cDNA encoding | 223 | 261 | −84 | −133 | 242 | −108.5 | −0.44835 | 2.2 |
| | rat cardionatrin precursor | | | | | | | | |
| J00771_at | J00771 RATPRNASE Rat | −50 | −13 | 430 | 353 | −31.5 | 391.5 | −12.4286 | at least |
| | pancreatic ribonuclease | | | | | | | | 2 fold |
| | mRNA | | | | | | | | |
| J05167_at | J05167 Rat band 3 Cl-/ | 512 | 412 | 164 | 159 | 462 | 161.5 | 0.349567 | −2.9 |
| | HCO₃ exchanger (B3RP3) | | | | | | | | |
| | mRNA, complete cds/ | | | | | | | | |
| | cds = 34.3717/gb = J05167/ | | | | | | | | |
| | gi = 203088/ug = Rn.9859/ | | | | | | | | |
| | len = 3877 | | | | | | | | |
| K00996mRNA_s_at | K00996mRNA RATCYP45E | 200 | 236 | 386 | 368 | 218 | 377 | 1.729358 | 1.7 |
| | Rat cytochrome p-450e | | | | | | | | |
| | (phenobarbital-induced) | | | | | | | | |
| | mRNA, 3′ end | | | | | | | | |
| L07380_g_at | L07380 RATGHRFRG | 375 | 435 | 236 | 227 | 405 | 231.5 | 0.571605 | −1.7 |
| | Rattus rattus (clone pGR2) | | | | | | | | |
| | growth hormone-releasing | | | | | | | | |
| | factor receptor mRNA | | | | | | | | |
| | sequence | | | | | | | | |
| L07398_at | L07398 RATIGVCL Rattus | 723 | 611 | 290 | 280 | 667 | 285 | 0.427286 | −2.3 |
| | norvegicus (hybridoma | | | | | | | | |
| | 56R-3) immunoglobulin | | | | | | | | |
| | rearranged gamma-chain | | | | | | | | |
| | mRNA variable (V) region, | | | | | | | | |
| | partial cds | | | | | | | | |
| M10140_at | M10140 Rat skeletal muscle | 43 | 81 | 345 | 410 | 62 | 377.5 | 6.08871 | 6.1 |
| | creatine kinase composite | | | | | | | | |
| | mRNA, complete cds/ | | | | | | | | |
| | cds = 69.1214/gb = M10140/ | | | | | | | | |
| | gi = 203477/ug = Rn.10756/ | | | | | | | | |
| | len = 1410 | | | | | | | | |
| M32754cds_s_at | M32754cds RATINHBAB1 | 297 | 256 | 578 | 655 | 276.5 | 616.5 | 2.229656 | 2.2 |
| | Rat inhibin alpha-subunit | | | | | | | | |
| | gene, exon 1 | | | | | | | | |
| M80826_at | M80826 Rat intestinal trefoil | 790 | 787 | 70 | −10 | 788.5 | 30 | 0.038047 | −26.3 |
| | protein mRNA, complete | | | | | | | | |
| | cds/cds = 17.262/ | | | | | | | | |
| | gb = M80826/gi = 207446/ | | | | | | | | |
| | ug = Rn.9960/len = 431 | | | | | | | | |
| rc_AI639304_at | Rat mixed-tissue library | 573 | 503 | 301 | 325 | 538 | 313 | 0.581784 | −1.7 |
| | Rattus norvegicus cDNA | | | | | | | | |
| | clone rx00157 3′, mRNA | | | | | | | | |
| | sequence [Rattus | | | | | | | | |
| | norvegicus] | | | | | | | | |
| rc_AA799581_at | rc_AA799581 EST189078 | 429 | 462 | 209 | 179 | 445.5 | 194 | 0.435466 | −2.3 |
| | Rattus norvegicus cDNA, 3′ | | | | | | | | |
| | end/clone = RHEAC77/ | | | | | | | | |
| | clone_end = 3′/ | | | | | | | | |
| | gb = AA799581/gi = 2862536/ | | | | | | | | |
| | ug = Rn.6207/len = 569 | | | | | | | | |
| rc_AA800211_at | rc_AA800211 EST189708 | 164 | 224 | 326 | 400 | 194 | 363 | 1.871134 | 1.9 |
| | Rattus norvegicus cDNA, 3′ | | | | | | | | |
| | end/clone = RHEAM49/ | | | | | | | | |
| | clone_end = 3′/ | | | | | | | | |
| | gb = AA800211/gi = 2863166/ | | | | | | | | |
| | ug = Rn.6299/len = 740 | | | | | | | | |
| rc_AA800549_at | rc_AA800549 EST190046 | 306 | 333 | 461 | 655 | 319.5 | 558 | 1.746479 | 1.7 |
| | Rattus norvegicus cDNA, 3′ | | | | | | | | |
| | end/clone = RLUAB29/ | | | | | | | | |
| | clone_end = 3′/ | | | | | | | | |
| | gb = AA800549/gi = 2863504/ | | | | | | | | |
| | ug = Rn.22957/len = 491 | | | | | | | | |
| rc_AA800749_at | rc_AA800749 EST190246 | 532 | 392 | 234 | 193 | 462 | 213.5 | 0.462121 | −2.2 |
| | Rattus norvegicus cDNA, 3′ | | | | | | | | |
| | end/clone = RLUAL02/ | | | | | | | | |
| | clone_end = 3′/ | | | | | | | | |
| | gb = AA800749/gi = 2863704/ | | | | | | | | |
| | ug = Rn.1897/len = 637 | | | | | | | | |
| rc_AA859680_g_at | rc_AA859680 UI-R-E0-bs-d- | 2002 | 1841 | 944 | 761 | 1921.5 | 852.5 | 0.443664 | −2.3 |
| | 12-0-UI.s1 Rattus | | | | | | | | |
| | norvegicus cDNA, 3′ end/ | | | | | | | | |
| | clone = UI-R-E0-bs-d-12-0- | | | | | | | | |
| | UI/clone_end = 3′/ | | | | | | | | |
| | gb = AA859680/gi = 2949200/ | | | | | | | | |
| | ug = Rn.22632/len = 437 | | | | | | | | |
| rc_AA874874_at | rc_AA874874 UI-R-E0-ci-d- | 761 | 632 | 1099 | 1322 | 696.5 | 1210.5 | 1.737976 | 1.7 |
| | 12-0-UI.s1 Rattus | | | | | | | | |
| | norvegicus cDNA, 3′ end/ | | | | | | | | |
| | clone = UI-R-E0-ci-d-12-0- | | | | | | | | |
| | UI/clone_end = 3′/ | | | | | | | | |
| | gb = AA874874/gi = 2979822/ | | | | | | | | |
| | ug = Rn.3157/len = 513 | | | | | | | | |
| rc_AA874919_at | rc_AA874919 UI-R-E0-ck-g- | 541 | 490 | 216 | 226 | 515.5 | 221 | 0.42871 | −2.3 |
| | 09-0-UI.s1 Rattus | | | | | | | | |
| | norvegicus cDNA, 3′ end/ | | | | | | | | |
| | clone = UI-R-E0-ck-g-09-0- | | | | | | | | |
| | UI/clone_end = 3′/ | | | | | | | | |
| | gb = AA874919/gi = 2979867/ | | | | | | | | |
| | ug = Rn.3174/len = 542 | | | | | | | | |
| rc_AA875127_g_at | rc_AA875127 UI-R-E0-bu- | 395 | 382 | 208 | 199 | 388.5 | 203.5 | 0.52381 | −1.9 |
| | d-05-0-UI.s2 Rattus | | | | | | | | |
| | norvegicus cDNA, 3′ end/ | | | | | | | | |
| | clone = UI-R-E0-bu-d-05-0- | | | | | | | | |
| | UI/clone_end = 3′/ | | | | | | | | |
| | gb = AA875127/gi = 2980075/ | | | | | | | | |
| | ug = Rn.18698/len = 579 | | | | | | | | |
| rc_AA891690_at | rc_AA891690 EST195493 | 167 | 189 | 391 | 335 | 178 | 363 | 2.039326 | 2.0 |
| | Rattus norvegicus cDNA, 3′ | | | | | | | | |
| | end/clone = RKIAF58/ | | | | | | | | |
| | clone_end = 3′/ | | | | | | | | |
| | gb = AA891690/gi = 3018569/ | | | | | | | | |
| | ug = Rn.22701/len = 446 | | | | | | | | |
| rc_AA891940_at | rc_AA891940 EST195743 | 109 | 29 | 385 | 426 | 69 | 405.5 | 5.876812 | 5.9 |
| | Rattus norvegicus cDNA, 3′ | | | | | | | | |
| | end/clone = RKIAI82/ | | | | | | | | |
| | clone_end = 3′/ | | | | | | | | |
| | gb = AA891940/gi = 3018819/ | | | | | | | | |
| | ug = Rn.3508/len = 523 | | | | | | | | |
| rc_AA892378_g_at | rc_AA892378 EST196181 | 959 | 890 | 1732 | 1866 | 924.5 | 1799 | 1.945917 | 1.9 |
| | Rattus norvegicus cDNA, 3′ | | | | | | | | |
| | end/clone = RKIAP70/ | | | | | | | | |
| | clone_end = 3′/ | | | | | | | | |
| | gb = AA892378/gi = 3019257/ | | | | | | | | |
| | ug = Rn.1298/len = 589 | | | | | | | | |
| rc_AA944423_at | rc_AA944423 EST199922 | 435 | 376 | 255 | 200 | 405.5 | 227.5 | 0.561036 | −1.8 |
| | Rattus norvegicus cDNA, 3′ | | | | | | | | |
| | end/clone = REMAJ02/ | | | | | | | | |
| | clone_end = 3′/ | | | | | | | | |
| | gb = AA944423/gi = 3104339/ | | | | | | | | |
| | ug = Rn.6165/len = 670 | | | | | | | | |
| rc_AA946384_at | rc_AA946384 EST201883 | 464 | 624 | 352 | 278 | 544 | 315 | 0.579044 | −1.7 |
| | Rattus norvegicus cDNA, 3′ | | | | | | | | |
| | end/clone = RLUBH49/ | | | | | | | | |
| | clone_end = 3′/ | | | | | | | | |
| | gb = AA946384/gi = 3106300/ | | | | | | | | |
| | ug = Rn.11301/len = 576 | | | | | | | | |
| rc_AI102868_g_at | rc_AI102868 EST212157 | 1431 | 1441 | 702 | 953 | 1436 | 827.5 | 0.576253 | −1.7 |
| | Rattus norvegicus cDNA, 3′ | | | | | | | | |
| | end/clone = REMBT90/ | | | | | | | | |
| | clone_end = 3′/gb = AI102868/ | | | | | | | | |
| | ug = Rn.221/len = 489 | | | | | | | | |
| rc_AI228599_at | rc_AI228599 EST225294 | 295 | 395 | 68 | 42 | 345 | 55 | 0.15942 | −6.3 |
| | Rattus norvegicus cDNA, 3′ | | | | | | | | |
| | end/clone = RBRCW95/ | | | | | | | | |
| | clone_end = 3′/gb = AI228599/ | | | | | | | | |
| | ug = Rn.3877/len = 572 | | | | | | | | |
| rc_AI236484_at | rc_AI236484 EST233046 | 124 | 115 | 247 | 263 | 119.5 | 255 | 2.133891 | 2.1 |
| | Rattus norvegicus cDNA, 3′ | | | | | | | | |
| | end/clone = ROVDG74/ | | | | | | | | |
| | clone_end = 3′/gb = AI236484/ | | | | | | | | |
| | ug = Rn.3924/len = 474 | | | | | | | | |
| rc_H31351_at | rc_H31351 EST105310 | 437 | 382 | 265 | 188 | 409.5 | 226.5 | 0.553114 | −1.8 |
| | Rattus norvegicus cDNA, 3′ | | | | | | | | |
| | end/clone = RPCAH85/ | | | | | | | | |
| | clone_end = 3′/ | | | | | | | | |
| | gb = H31351/gi = 976768/ | | | | | | | | |
| | ug = Rn.14564/len = 352 | | | | | | | | |
| S70803_g_at | S70803 clone p10.15 | 584 | 699 | 147 | 199 | 641.5 | 173 | 0.26968 | −3.7 |
| | product [rats, osteosarcoma | | | | | | | | |
| | ROS17/2.8, mRNA, 737 nt] | | | | | | | | |
| U01146_s_at | U01146 RRU01146 Rattus | 432 | 367 | 586 | 799 | 399.5 | 692.5 | 1.733417 | 1.7 |
| | rattus Sprague Dawley | | | | | | | | |
| | nuclear orphan receptor | | | | | | | | |
| | HZF-3 (HZF-3) mRNA, | | | | | | | | |
| | complete cds | | | | | | | | |
| U14192complete_seq_at | U14192completeSeq Rattus | 311 | 292 | 163 | 168 | 301.5 | 165.5 | 0.548922 | −1.8 |
| | norvegicus general | | | | | | | | |
| | vesicular transport factor | | | | | | | | |
| | p115 mRNA, complete cds/ | | | | | | | | |
| | cds = 11.2890/ | | | | | | | | |
| | gb = U14192/gi = 538152/ | | | | | | | | |
| | ug = Rn.4746/len = 2891 | | | | | | | | |
| X03347cds_g_at | X03347cds REMSVFBR | 232 | 304 | 461 | 496 | 268 | 478.5 | 1.785448 | 1.8 |
| | FBR-murine osteosarcoma | | | | | | | | |
| | provirus genome | | | | | | | | |
| X12554cds_s_at | X12554cds RNCOX6AH | 269 | 222 | 401 | 470 | 245.5 | 435.5 | 1.773931 | 1.8 |
| | Rat mRNA for heart | | | | | | | | |
| | cytochrome c oxidase | | | | | | | | |
| | subunit Via | | | | | | | | |
| X63446_at | X63446 R. norvegicus | 520 | 388 | 248 | 249 | 454 | 248.5 | 0.547357 | −1.8 |
| | mRNA for fetuin/ | | | | | | | | |
| | cds = 31,1089/gb = X63446/ | | | | | | | | |
| | gi = 56139/ug = Rn.3880/ | | | | | | | | |
| | len = 1456 | | | | | | | | |

Genes that passed the filtering criteria outlined above for differential expression between 1 week extinction and 1 week withdrawal in the CeA. Average difference values (from GeneChip version 3.2) are listed for each gene from all groups. Affymetrix probe set numbers are listed along with the common name of the genes, if known.

TABLE 6
CeA 1-Week Withdrawal to Control

| | | | | | 1-week | | | | |
|---|---|---|---|---|---|---|---|---|---|
| | | | 1-week | 1-week | with- | | | | |
| | | 1-week | with- | extinction | drawal | Mean | Mean | | Fold |
| Probe set no. | Description | extinction | drawal | control | control | control | extinction | Ratio | change |
| AB003753cds#1_at | AB003753cds#1 Rattus | 373 | 366 | 82 | 126 | 369.5 | 104 | 0.281461 | −3.6 |
| | norvegicus genes for | | | | | | | | |
| | high sulfur protein B2E | | | | | | | | |
| | and high sulfur protein | | | | | | | | |
| | B2F, complete cds | | | | | | | | |
| AB015433_s_at | AB015433 Rattus | 269 | 253 | 609 | 524 | 261 | 566.5 | 2.170498 | 2.2 |
| | norvegicus mRNA for 4F2 | | | | | | | | |
| | heavy chain (4F2hc), | | | | | | | | |
| | complete cds | | | | | | | | |
| AB016160_g_at | AB016160 Rattus | 414 | 318 | 148 | 150 | 366 | 149 | 0.407104 | −2.5 |
| | norvegicus mRNA for | | | | | | | | |
| | GABAB receptor 1c, | | | | | | | | |
| | complete cds | | | | | | | | |
| AF063302mRNA#3_s_at | AF063302mRNA#3 Rattus | 421 | 395 | 140 | −4 | 408 | 68 | 0.166667 | −6.0 |
| | norvegicus carnitine | | | | | | | | |
| | palmitoyltransferase | | | | | | | | |
| | Ibeta 1, carnitine | | | | | | | | |
| | palmitoyltransferase | | | | | | | | |
| | Ibeta 2, and carnitine | | | | | | | | |
| | palmitoyltransferase | | | | | | | | |
| | Ibeta 3 gene, nuclear | | | | | | | | |
| | gene encoding mito- | | | | | | | | |
| | chondrial proteins, | | | | | | | | |
| | alternatively spliced | | | | | | | | |
| | products, partial cds | | | | | | | | |
| AF064856_at | AF064856 Rattus sp. | 561 | 529 | 332 | 244 | 545 | 288 | 0.52844 | −1.9 |
| | 7acomp protein mRNA, | | | | | | | | |
| | complete cds | | | | | | | | |
| AF081144_s_at | AF081144 Rattus | 288 | 202 | 495 | 578 | 245 | 536.5 | 2.189796 | 2.2 |
| | norvegicus CL1AA mRNA, | | | | | | | | |
| | complete cds | | | | | | | | |
| D10853_at | D10853 RATATR Rat | 240 | 226 | 119 | 115 | 233 | 117 | 0.502146 | −2.0 |
| | mRNA for amidophos- | | | | | | | | |
| | phoribosyltransferase | | | | | | | | |
| D13309_s_at | D13309 RATRDBPB Rat | 626 | 625 | 348 | 359 | 625.5 | 353.5 | 0.565148 | −1.8 |
| | mRNA for DNA-binding | | | | | | | | |
| | protein B | | | | | | | | |
| D64085_at | D64085 RATORFA1 Rat | 443 | 344 | 114 | 245 | 393.5 | 179.5 | 0.456163 | −2.2 |
| | mRNA for fibroblast | | | | | | | | |
| | growth factor FGF-5, | | | | | | | | |
| | complete cds | | | | | | | | |
| D83538_g_at | D83538 Rat mRNA for | 178 | 202 | 386 | 470 | 190 | 428 | 2.252632 | 2.3 |
| | 230 kDa phosphatidyli- | | | | | | | | |
| | nositol 4-kinase, | | | | | | | | |
| | complete cds/ | | | | | | | | |
| | cds = 391.6516/gb = D83538/ | | | | | | | | |
| | gi = 1339965/ug = Rn.11015/ | | | | | | | | |
| | len = 6857 | | | | | | | | |
| J00771_at | J00771 RATPRNASE Rat | 262 | 238 | −50 | −13 | 250 | −31.5 | −0.126 | 7.9 |
| | pancreatic ribo- | | | | | | | | |
| | nuclease mRNA | | | | | | | | |
| L07398_at | L07398 RATIGVCL | 305 | 311 | 723 | 611 | 308 | 667 | 2.165584 | 2.2 |
| | Rattus norvegicus | | | | | | | | |
| | (hybridoma 56R-3) | | | | | | | | |
| | immunoglobulin re- | | | | | | | | |
| | arranged gamma-chain | | | | | | | | |
| | mRNA variable (V) | | | | | | | | |
| | region, partial cds | | | | | | | | |
| L19699_at | L19699 Rat GTP-binding | 331 | 276 | 657 | 711 | 303.5 | 684 | 2.253707 | 2.3 |
| | protein (ral B) mRNA, | | | | | | | | |
| | complete cds/ | | | | | | | | |
| | cds = 64.684/gb = L19699/ | | | | | | | | |
| | gi = 310211/ug = Rn.4586/ | | | | | | | | |
| | len = 2074 | | | | | | | | |
| L40364_f_at | L40364 Rattus | 177 | 129 | 475 | 403 | 153 | 439 | 2.869281 | 2.9 |
| | norvegicus MHC class | | | | | | | | |
| | I RT1.O type - 149 | | | | | | | | |
| | processed pseudogene | | | | | | | | |
| | mRNA/cds = UNKNOWN/ | | | | | | | | |
| | gb = L40364/gi = 992568/ | | | | | | | | |
| | ug = Rn.3577/len = 1602 | | | | | | | | |
| M55050_at | M55050 Rattus norwegicus | 533 | 378 | 231 | 237 | 455.5 | 234 | 0.513721 | −1.9 |
| | interleukin-2 receptor | | | | | | | | |
| | beta chain (p70/75) | | | | | | | | |
| | mRNA, complete cds/ | | | | | | | | |
| | cds = 111,1724/ | | | | | | | | |
| | gb = M55050/gi = 204913/ | | | | | | | | |
| | ug = Rn.5832/len = 2598 | | | | | | | | |
| M81639_at | M81639 Rattus norvegicus | 292 | 316 | 474 | 592 | 304 | 533 | 1.753289 | 1.8 |
| | stannin mRNA/ | | | | | | | | |
| | cds = UNKNOWN/ | | | | | | | | |
| | gb = M81639/gi = 207078/ | | | | | | | | |
| | ug = Rn.6147/len = 2897 | | | | | | | | |
| rc_AI639096_at | Rat mixed-tissue library | 111 | 239 | 392 | 383 | 175 | 387.5 | 2.214286 | 2.2 |
| | Rattus norvegicus cDNA | | | | | | | | |
| | clone rx00904 3′, mRNA | | | | | | | | |
| | sequence [Rattus | | | | | | | | |
| | norvegicus] | | | | | | | | |
| rc_AI639391_at | Rat mixed-tissue library | 982 | 1009 | 284 | 334 | 995.5 | 309 | 0.310397 | −3.2 |
| | Rattus norvegicus cDNA | | | | | | | | |
| | clone rx02754 3′, mRNA | | | | | | | | |
| | sequence [Rattus | | | | | | | | |
| | norvegicus] | | | | | | | | |
| rc_AI638980_at | Rat mixed-tissue library | 631 | 601 | 277 | 221 | 616 | 249 | 0.404221 | −2.5 |
| | Rattus norvegicus cDNA | | | | | | | | |
| | clone rx03968 3′, mRNA | | | | | | | | |
| | sequence [Rattus | | | | | | | | |
| | norvegicus] | | | | | | | | |
| rc_AI639195_r_at | Rat mixed-tissue library | 822 | 933 | 519 | 393 | 877.5 | 456 | 0.519658 | −1.9 |
| | Rattus norvegicus cDNA | | | | | | | | |
| | clone rx04881 3′, mRNA | | | | | | | | |
| | sequence [Rattus | | | | | | | | |
| | norvegicus] | | | | | | | | |
| rc_AA799421_at | rc_AA799421 EST188918 | 359 | 319 | 479 | 675 | 339 | 577 | 1.702065 | 1.7 |
| | Rattus norvegicus | | | | | | | | |
| | cDNA, 3′ end/ | | | | | | | | |
| | clone = RHEAA87/ | | | | | | | | |
| | clone_end = 3′/ | | | | | | | | |
| | gb = AA799421/gi = 2862376/ | | | | | | | | |
| | ug = Rn.19951/len = 570 | | | | | | | | |
| rc_AA799449_g_at | rc_AA799449 EST188946 | 262 | 327 | 470 | 670 | 294.5 | 570 | 1.935484 | 1.9 |
| | Rattus norvegicus | | | | | | | | |
| | cDNA, 3′ end/ | | | | | | | | |
| | clone = RHEAB19/ | | | | | | | | |
| | clone_end = 3′/ | | | | | | | | |
| | gb = AA799449/gi = 2862404/ | | | | | | | | |
| | ug = Rn.3286/len = 553 | | | | | | | | |
| rc_AA799671_at | rc_AA799671 EST189168 | 421 | 529 | 249 | 297 | 475 | 273 | 0.574737 | −1.7 |
| | Rattus norvegicus | | | | | | | | |
| | cDNA, 3′ end/ | | | | | | | | |
| | clone = RHEAD82/ | | | | | | | | |
| | clone_end = 3′/ | | | | | | | | |
| | gb = AA799671/gi = 2862626/ | | | | | | | | |
| | ug = Rn.6219/len = 328 | | | | | | | | |
| rc_AA799899_i_at | rc_AA799899 EST189396 | 4497 | 3805 | 6266 | 7851 | 4151 | 7058.5 | 1.700434 | 1.7 |
| | Rattus norvegicus | | | | | | | | |
| | cDNA, 3′ end/ | | | | | | | | |
| | clone = RHEAG67/ | | | | | | | | |
| | clone_end = 3′/ | | | | | | | | |
| | gb = AA799899/gi = 2862854/ | | | | | | | | |
| | ug = Rn.5974/len = 505 | | | | | | | | |
| rc_AA859680_g_at | rc_AA859680 UI-R-E0-bs- | 731 | 959 | 2002 | 1841 | 845 | 1921.5 | 2.273964 | 2.3 |
| | d-12-0-UI.s1 Rattus | | | | | | | | |
| | norvegicus cDNA, 3′ end/ | | | | | | | | |
| | clone = UI-R-E0-bs-d-12- | | | | | | | | |
| | 0-UI/clone_end = 3′/ | | | | | | | | |
| | gb = AA859680/gi = 2949200/ | | | | | | | | |
| | ug = Rn.22632/len = 437 | | | | | | | | |
| rc_AA875054_at | rc_AA875054 UI-R-E0- | 779 | 581 | 320 | 455 | 680 | 387.5 | 0.569853 | −1.8 |
| | cb-e-04-0-UI.s1 Rattus | | | | | | | | |
| | norvegicus cDNA, 3′ end/ | | | | | | | | |
| | clone = UI-R-E0-cb-e-04- | | | | | | | | |
| | 0-UI/clone_end = 3′/ | | | | | | | | |
| | gb = AA875054/gi = 2980002/ | | | | | | | | |
| | ug = Rn.24874/len = 485 | | | | | | | | |
| rc_AA891438_g_at | rc_AA891438 EST195241 | 557 | 438 | 58 | 235 | 497.5 | 146.5 | 0.294472 | −3.4 |
| | Rattus norvegicus | | | | | | | | |
| | cDNA, 3′ end/ | | | | | | | | |
| | clone = RHEAU25/ | | | | | | | | |
| | clone_end = 3′/ | | | | | | | | |
| | gb = AA891438/gi = 3018317/ | | | | | | | | |
| | ug = Rn.22406/len = 397 | | | | | | | | |
| rc_AA891690_at | rc_AA891690 EST195493 | 316 | 308 | 167 | 189 | 312 | 178 | 0.570513 | −1.8 |
| | Rattus norvegicus | | | | | | | | |
| | cDNA, 3′ end/ | | | | | | | | |
| | clone = RKIAF58/ | | | | | | | | |
| | clone_end = 3′/ | | | | | | | | |
| | gb = AA891690/gi =3018569/ | | | | | | | | |
| | ug = Rn.22701/len = 446 | | | | | | | | |
| rc_AA892859_at | rc_AA892859 EST196662 | 236 | 225 | −51 | −31 | 230.5 | −41 | −0.17787 | 5.6 |
| | Rattus norvegicus | | | | | | | | |
| | cDNA, 3′ end/ | | | | | | | | |
| | clone = RKIAY19/ | | | | | | | | |
| | clone_end = 3′/ | | | | | | | | |
| | gb = AA892859/gi = 3019738/ | | | | | | | | |
| | ug = Rn.8137/len = 568 | | | | | | | | |
| rc_AA899106_at | rc_AA899106 UI-R-E0-cw- | 550 | 698 | 170 | 185 | 624 | 177.5 | 0.284455 | −3.5 |
| | d-04-0-UI.s1 Rattus | | | | | | | | |
| | norvegicus cDNA, 3′ end/ | | | | | | | | |
| | clone = UI-R-E0-cw-d-04- | | | | | | | | |
| | 0-UI/clone_end = 3′/ | | | | | | | | |
| | gb = AA899106/gi = 3034460/ | | | | | | | | |
| | ug = Rn.6031/len = 523 | | | | | | | | |
| rc_AA944422_at | rc_AA944422 EST199921 | 109 | 240 | 382 | 519 | 174.5 | 450.5 | 2.581662 | 2.6 |
| | Rattus norvegicus | | | | | | | | |
| | cDNA, 3′ end/ | | | | | | | | |
| | clone = REMAJ01/ | | | | | | | | |
| | clone_end = 3′/ | | | | | | | | |
| | gb = AA944422/gi = 3104338/ | | | | | | | | |
| | ug = Rn.871/len = 641 | | | | | | | | |
| rc_AI060085_s_at | rc_AI060085 UI-R-C1-Ii- | 263 | 258 | 137 | 117 | 260.5 | 127 | 0.487524 | −2.1 |
| | c-08-0-UI.s1 Rattus | | | | | | | | |
| | norvegicus cDNA, 3′ end/ | | | | | | | | |
| | clone = UI-R-C1-Ii-c-08- | | | | | | | | |
| | 0-UI/clone_end = 3′/ | | | | | | | | |
| | gb = AI060085/ug = Rn.9967/ | | | | | | | | |
| | len = 315 | | | | | | | | |
| rc_AI138143_at | rc_AI138143 UI-R-C0-if- | 219 | 210 | 119 | 101 | 214.5 | 110 | 0.512821 | −2.0 |
| | e-07-0-UI.s1 Rattus | | | | | | | | |
| | norvegicus cDNA, 3′ end/ | | | | | | | | |
| | clone = UI-R-C0-if-e-07- | | | | | | | | |
| | 0-UI/clone_end = 3′/ | | | | | | | | |
| | gb = AI138143/ug = Rn.10708/ | | | | | | | | |
| | len = 343 | | | | | | | | |
| rc_AI170212_s_at | rc_AI170212 EST216137 | 271 | 280 | 552 | 626 | 275.5 | 589 | 2.137931 | 2.1 |
| | Rattus norvegicus cDNA, 3′ | | | | | | | | |
| | end/clone = RLUCF03/ | | | | | | | | |
| | clone_end = 3′/ | | | | | | | | |
| | gb = AI170212/gi = 3710252/ | | | | | | | | |
| | ug = Rn.11007/len = 322 | | | | | | | | |
| rc_AI170268_at | rc_AI170268 EST216194 | 361 | 290 | 492 | 620 | 325.5 | 556 | 1.708141 | 1.7 |
| | Rattus norvegicus cDNA, 3′ | | | | | | | | |
| | end/clone = RLUCG30/ | | | | | | | | |
| | clone_end = 3′/ | | | | | | | | |
| | gb = AI170268/gi = 3710308/ | | | | | | | | |
| | ug = Rn.1868/len = 577 | | | | | | | | |
| rc_AI176488_at | rc_AI176488 EST220073 | 300 | 391 | 188 | 27 | 345.5 | 107.5 | 0.311143 | −3.2 |
| | Rattus norvegicus cDNA, 3′ | | | | | | | | |
| | end/clone = ROVBS47/ | | | | | | | | |
| | clone_end = 3′/gb = AI176488/ | | | | | | | | |
| | ug = Rn.9909/len = 650 | | | | | | | | |
| rc_AI228599_at | rc_AI228599 EST225294 | −79 | −37 | 295 | 395 | −58 | 345 | −5.94828 | at least |
| | Rattus norvegicus cDNA, 3′ | | | | | | | | 2-fold |
| | end/clone = RBRCW95/ | | | | | | | | |
| | clone_end = 3′/gb = AI228599/ | | | | | | | | |
| | ug = Rn.3877/len = 572 | | | | | | | | |
| rc_AI231519_at | rc_AI231519 EST228207 | 175 | 180 | 403 | 361 | 177.5 | 382 | 2.152113 | 2.2 |
| | Rattus norvegicus cDNA, 3′ | | | | | | | | |
| | end/clone = REMDL26/ | | | | | | | | |
| | clone_end = 3′/gb = AI231519/ | | | | | | | | |
| | ug = Rn.6602/len = 482 | | | | | | | | |
| Rc_H33651_at | rc_H33651 EST109846 | 406 | 309 | 216 | 189 | 357.5 | 202.5 | 0.566434 | −1.8 |
| | Rattus norvegicus cDNA, 3′ | | | | | | | | |
| | end/clone = RPNAV67/ | | | | | | | | |
| | clone_end = 3′/ | | | | | | | | |
| | gb = H33651/gi = 979068/ | | | | | | | | |
| | ug = Rn.14654/len = 447 | | | | | | | | |
| U14414_at | U14414 Rattus norvegicus | 281 | 294 | 126 | 129 | 287.5 | 127.5 | 0.443478 | −2.3 |
| | P2x receptor mRNA, | | | | | | | | |
| | complete cds/cds = 36,1454/ | | | | | | | | |
| | gb = U14414/gi = 558830/ | | | | | | | | |
| | ug = Rn.10991/len = 1831 | | | | | | | | |
| U70270UTR#1_f_at | U70270UTR#1 RNMUD402 | 537 | 468 | −153 | 66 | 502.5 | −43.5 | −0.08657 | 11.6 |
| | Rattus norvegicus mud-4 | | | | | | | | |
| | mRNA, 3′ UTR | | | | | | | | |
| U75921UTR#1_at | U75921UTR#1 RNAPCBP3 | 412 | 388 | 122 | 181 | 400 | 151.5 | 0.37875 | −2.6 |
| | Rattus norvegicus APC | | | | | | | | |
| | binding protein EB1 | | | | | | | | |
| | mRNA, 3′ untranslated | | | | | | | | |
| | region, partial sequence | | | | | | | | |
| X03347cds_at | X03347cds REMSVFBR | 463 | 513 | 252 | 117 | 488 | 184.5 | 0.378074 | −2.6 |
| | FBR-murine osteosarcoma | | | | | | | | |
| | provirus genome | | | | | | | | |
| X12554cds_s_at | X12554cds RNCOX6AH | 544 | 449 | 269 | 222 | 496.5 | 245.5 | 0.494461 | −2.0 |
| | Rat mRNA for heart | | | | | | | | |
| | cytochrome c oxidase | | | | | | | | |
| | subunit VIa | | | | | | | | |
| X15679_at | X15679 Rat mRNA for | 707 | 595 | 371 | 362 | 651 | 366.5 | 0.56298 | −1.8 |
| | preprotrypsinogen IV (EC | | | | | | | | |
| | 3.4.21.4)/cds = 14,757/ | | | | | | | | |
| | gb = X15679/gi = 56813/ | | | | | | | | |
| | ug = Rn.10387/len = 862 | | | | | | | | |
| X60651mRNA_s_at | X60651mRNA RNSYNDCN | 407 | 374 | 169 | 191 | 390.5 | 180 | 0.460948 | −2.2 |
| | Rat mRNA for syndecan | | | | | | | | |
| X73579_at | X73579 R. norvegicus CD23 | −43 | 23 | 466 | 604 | −10 | 535 | −53.5 | at least |
| | mRNA/cds = 0.929/ | | | | | | | | 2-fold |
| | gb = X73579/gi = 313672/ | | | | | | | | |
| | ug = Rn.10326/len = 1146 | | | | | | | | |

Genes that passed the filtering criteria outlined above for differential expression between 1 week withdrawal and its corresponding control in the CeA. Average difference values (from GeneChip version 3.2) are listed for each gene from all groups. Affymetrix probe set numbers are listed along with the common name of the genes, if known.

TABLE 7
Core 1-Week Extinction to Control

| | | 1-week | 1-week | 1-week | 1-week | |
|---|---|---|---|---|---|---|
| | | extinction | extinction | extinction | extinction | Fold |
| Experiment | Description | control core | control core | core | core | change |
| K02248cds_s_at | K02248cds RATSOM141 Rat | 575 | 528 | 342 | 274 | −1.8 |
| | somatostatin-14 gene, complete cds | | | | | |
| M55534mRNA_s_at | M55534mRNA Rat alpha-crystallin B | 167 | 264 | 416 | 414 | 1.8 |
| | chain mRNA, complete cds/ | | | | | |
| | cds = UNKNOWN/gb = M55534/ | | | | | |
| | gi = 203609/ug = Rn.832/len = 1247 | | | | | |
| Rc_AA894296_at | rc_AA894296 EST198099 Rattus | 209 | 217 | 436 | 362 | 1.9 |
| | norvegicus cDNA, 3′ end/ | | | | | |
| | clone = RSPAW17/clone_end = 3′/ | | | | | |
| | gb = AA894296/gi = 3021175/ | | | | | |
| | ug = Rn.3760/len = 600 | | | | | |
| Rc_AI058941_s_at | rc_AI058941 UI-R-C1-Ir-b-07-0-UI.s1 | 222 | −3 | 389 | 372 | 1.8 |
| | Rattus norvegicus cDNA, 3′ end/ | | | | | |
| | clone = UI-R-C1-Ir-b-07-0-UI/ | | | | | |
| | clone_end = 3′/gb = AI058941/ | | | | | |
| | ug = Rn.4231/len = 476 | | | | | |
| X15679_at | X15679 Rat mRNA for | 353 | 365 | 201 | 120 | −1.8 |
| | preprotrypsinogen IV (EC 3.4.21.4)/ | | | | | |
| | cds = 14.757/gb = X15679/ | | | | | |
| | gi = 56813/ug = Rn.10387/len = 862 | | | | | |
| X95990exon_s_at | X95990exon RNC5ARECP | 645 | 544 | 328 | 360 | −1.7 |
| | R. norvegicus mRNA for C5a | | | | | |
| | anaphylatoxin receptor | | | | | |
| Z11581_at | Z11581 R. norvegicus mRNA for | 683 | 724 | 357 | 460 | −1.7 |
| | kainate receptor subunit (ka2)/ | | | | | |
| | cds = 202.3141/gb = Z11581/gi = 56509/ | | | | | |
| | ug = Rn.10053/len = 3702 | | | | | |
| U05013_at | U05013 Rattus norvegicus Sprague- | 209 | 241 | 48 | 53 | 4.4 |
| | Dawley heme oxygenase-2 non- | | | | | |
| | reducing isoform gene, complete cds/ | | | | | |
| | cds = 177.1124/gb = U05013/ | | | | | |
| | gi = 501034/ug = Rn.10241/len = 1815 | | | | | |
| M64785_g_at | M64785 RATVAS Rat vasopressin (VP) | 200 | 211 | 116 | 110 | 1.8 |
| | mRNA | | | | | |

Genes that passed the filtering criteria outlined above for differential expression between 1 week extinction and its corresponding control in the nucleus accumbens core. Average difference values (from GeneChip version 3.2) are listed for each gene from all groups. Affymetrix probe set numbers are listed along with the common name of the genes, if known.

TABLE 8
Core 1-Week Extinction to Withdrawal

| | | 1-week | 1-week | 1-week | 1-week | |
|---|---|---|---|---|---|---|
| | | withdrawal | withdrawal | extinction | extinction | Fold |
| Experiment | Description | B | A | B | A | change |
| AF055714UTR#1_at | AF055714UTR#1 Rattus norvegicus | 481 | 466 | 2 | −17 | −2.4 |
| | hypertension-regulated vascular factor- | | | | | |
| | 1C-4 mRNA, 3′ UTR | | | | | |
| AF102855_at | AF102855 Rattus norvegicus synaptic | 238 | 264 | 110 | 109 | 2 |
| | SAPAP-interacting protein Synamon | | | | | |
| | mRNA, complete cds | | | | | |
| M11071_f_at | M11071 Rat MHC class I cell surface | 1021 | 897 | 2204 | 1642 | 2.0 |
| | antigen mRNA/cds = 0,330/gb = M11071/ | | | | | |
| | gi = 205414/ug = Rn.11168/len = 824 | | | | | |
| M25890_at | M25890 Rat somatostatin mRNA, | 875 | 668 | 1269 | 1448 | 1.8 |
| | complete cds/cds = 60.410/gb = M25890/ | | | | | |
| | gi = 207030/ug = Rn.540/len = 564 | | | | | |
| M92076_at | M92076 RATMGLURC Rat | 256 | 359 | 709 | 668 | 2.2 |
| | metabotropic glutamate receptor 3 | | | | | |
| | Mrna, primary transcript | | | | | |
| M95591_g_at | M95591 RATSST Rattus rattus hepatic | 472 | 494 | 141 | 235 | −2.2 |
| | squalene synthetase mRNA, complete | | | | | |
| | cds | | | | | |
| M96626_g_at | M96626 RAT plasma membrane CA2+− | 206 | 222 | 96 | 76 | 2 |
| | ATPase isoform 3 mRNA, partial cds/ | | | | | |
| | cds = 0.346/gb = M96626/gi = 203212/ | | | | | |
| | ug = Rn.11053/len = 609 | | | | | |
| rc_AI638989_at | Rat mixed-tissue library Rattus | 168 | 135 | 451 | 368 | 2.0 |
| | norvegicus cDNA clone rx01268 3′, | | | | | |
| | mRNA sequence [Rattus norvegicus] | | | | | |
| rc_AA819776_f_at | rc_AA819776 UI-R-A0-ap-h-07-0-UI.s1 | 56 | −42 | 471 | 384 | 2.1 |
| | UI-R-A0 Rattus norvegicus cDNA clone | | | | | |
| | UI-R-A0-ap-h-07-0-UI 3′ similar to | | | | | |
| | gb\|J04633\|MUSHSP86A Mouse heat | | | | | |
| | shock protein 86 mRNA, complete cds, | | | | | |
| | and 28S ribosomal RNA, partial | | | | | |
| | sequence, mRNA sequence [Rattus | | | | | |
| | norvegicus] | | | | | |
| rc_AA858621_g_at | rc_AA858621 UI-R-E0-bq-b-10-0-UI.s1 | 439 | 335 | 691 | 870 | 2.0 |
| | Rattus norvegicus cDNA, 3′ end/ | | | | | |
| | clone = UI-R-E0-bq-b-10-0-UI/ | | | | | |
| | clone_end = 3′/gb = AA858621/ | | | | | |
| | gi = 2948961/ug = Rn.3551/len = 550 | | | | | |
| rc_AA859520_at | rc_AA859520 UI-R-E0-br-b-02-0-UI.s1 | 230 | 297 | 535 | 507 | 2.0 |
| | Rattus norvegicus cDNA, 3′ end/ | | | | | |
| | clone = UI-R-E0-br-b-02-0-UI/ | | | | | |
| | clone_end = 3′/gb = AA859520/ | | | | | |
| | gi = 2949040/ug = Rn.23034/len = 453 | | | | | |
| rc_AA859966_i_at | rc_AA859966 UI-R-E0-ca-g-03-0-UI.s1 | −129 | −223 | 5469 | 5453 | 27.3 |
| | Rattus norvegicus cDNA, 3′ end/ | | | | | |
| | clone = UI-R-E0-ca-g-03-0-UI/ | | | | | |
| | clone_end = 3′/gb = AA859966/ | | | | | |
| | gi = 2949486/ug = Rn.861/len = 392 | | | | | |
| rc_AA875103_at | rc_AA875103 UI-R-E0-cf-h-04-0-UI.s1 | 299 | 266 | −20 | −49 | 14 |
| | Rattus norvegicus cDNA, 3′ end/ | | | | | |
| | clone = UI-R-E0-cf-h-04-0-UI/ | | | | | |
| | clone_end = 3′/gb = AA875103/ | | | | | |
| | gi = 2980051/ug = Rn.22643/len = 606 | | | | | |
| rc_AA875131_at | rc_AA875131 UI-R-E0-bu-e-03-0-UI.s2 | 381 | 429 | 186 | 231 | −1.9 |
| | Rattus norvegicus cDNA, 3′ end/ | | | | | |
| | clone = UI-R-E0-bu-e-03-0-UI/ | | | | | |
| | clone_end = 3′/gb = AA875131/ | | | | | |
| | gi = 2980079/ug = Rn.2801/len = 575 | | | | | |
| rc_AA891721_at | rc_AA891721 EST195524 Rattus | 342 | 417 | 166 | 170 | −1.9 |
| | norvegicus cDNA, 3′ end/ | | | | | |
| | clone = RKIAF94/clone_end = 3′/ | | | | | |
| | gb = AA891721/gi = 3018600/ | | | | | |
| | ug = Rn.14709/len = 454 | | | | | |
| rc_AA893065_at | rc_AA893065 EST196868 Rattus | 225 | 254 | 516 | 489 | 2.1 |
| | norvegicus cDNA, 3′ end/ | | | | | |
| | clone = RKIBB69/clone_end = 3′/ | | | | | |
| | gb = AA893065/gi = 3019944/ | | | | | |
| | ug = Rn.13472/len = 410 | | | | | |
| rc_AA893612_at | rc_AA893612 EST197415 Rattus | 517 | 514 | 942 | 919 | 1.8 |
| | norvegicus cDNA, 3′ end/ | | | | | |
| | clone = RPLAC57/clone_end = 3′/ | | | | | |
| | gb = AA893612/gi = 3020491/ | | | | | |
| | ug = Rn.14814/len = 265 | | | | | |
| rc_AA893870_g_at | rc_AA893870 EST197673 Rattus | 46 | 62 | 308 | 316 | 6 |
| | norvegicus cDNA, 3′ end/ | | | | | |
| | clone = RPLAM86/clone_end = 3′/ | | | | | |
| | gb = AA893870/gi = 3020749/ | | | | | |
| | ug = Rn.11229/len = 417 | | | | | |
| rc_AA945054_s_at | rc_AA945054 EST200553 Rattus | 449 | 573 | 801 | 975 | 1.7 |
| | norvegicus cDNA, 3′ end/ | | | | | |
| | clone = RLIAF82/clone_end = 3′/ | | | | | |
| | gb = AA945054/ug = Rn.1055/len = 565 | | | | | |
| rc_AA955983_at | rc_AA955983 UI-R-E1-fb-e-12-0-UI.s1 | 579 | 704 | 351 | 398 | −1.7 |
| | Rattus norvegicus cDNA, 3′ end/ | | | | | |
| | clone = UI-R-E1-fb-e-12-0-UI/ | | | | | |
| | clone_end = 3′/gb = AA955983/ | | | | | |
| | ug = Rn.7854/len = 542 | | | | | |
| rc_AI008863_at | rc_AI008863 EST203314 Rattus | 322 | 450 | 196 | 245 | −1.7 |
| | norvegicus cDNA, 3′ end/ | | | | | |
| | clone = REMBE50/clone_end = 3′/ | | | | | |
| | gb = AI008863/ug = Rn.1893/len = 401 | | | | | |
| rc_AI013194_at | rc_AI013194 EST207869 Rattus | 217 | 251 | 584 | 531 | 2 |
| | norvegicus cDNA, 3′ end/ | | | | | |
| | clone = RSPBH90/clone_end = 3′/ | | | | | |
| | gb = AI013194/ug = Rn.3506/len = 464 | | | | | |
| rc_AI014135_g_at | rc_AI014135 EST207690 Rattus | 1499 | 1401 | 567 | 444 | 3 |
| | norvegicus cDNA, 3′ end/ | | | | | |
| | clone = RSPBF48/clone_end = 3′/ | | | | | |
| | gb = AI014135/ug = Rn.4229/len = 410 | | | | | |
| rc_AI102103_at | rc_AI102103 EST211392 Rattus | 1193 | 1211 | 698 | 655 | −1.8 |
| | norvegicus cDNA, 3′ end/ | | | | | |
| | clone = RBRBY91/clone_end = 3′/ | | | | | |
| | gb = AI102103/gi = 3706936/ | | | | | |
| | ug = Rn.14991/len = 611 | | | | | |
| rc_AI172097_g_at | rc_AI172097 EST218092 Rattus | 274 | 323 | 541 | 556 | 1.8 |
| | norvegicus cDNA, 3′ end/ | | | | | |
| | clone = RMUBU88/clone_end = 3′/ | | | | | |
| | gb = AI172097/gi = 3712137/ | | | | | |
| | ug = Rn.20418/len = 570 | | | | | |
| rc_H31982_at | rc_H31982 EST106584 Rattus | 354 | 431 | 170 | 175 | −2.0 |
| | norvegicus cDNA, 3′ end/ | | | | | |
| | clone = RPCBE17/clone_end = 3′/ | | | | | |
| | gb = H31982/gi = 977399/ug = Rn.7138/ | | | | | |
| | len = 363 | | | | | |
| U62897_at | U62897 Rattus norvegicus | 183 | 216 | 344 | 435 | 1.9 |
| | carboxypeptidase D precursor (Cpd) | | | | | |
| | mRNA, complete cds/cds = 45.4181/ | | | | | |
| | gb = U62897/gi = 2406562/ | | | | | |
| | ug = Rn.4093/len = 4377 | | | | | |
| U67995_s_at | U67995 Rattus norvegicus stearyl-CoA | 1336 | 1291 | 780 | 591 | −1.9 |
| | desaturase 2 mRNA, partial cds/ | | | | | |
| | cds = 0.92/gb = U67995/gi = 1763026/ | | | | | |
| | ug = Rn.10650/len = 315 | | | | | |
| U77931_at | U77931 RNU77931 Rattus norvegicus | 836 | 912 | 2166 | 1850 | 2.3 |
| | unknown mRNA | | | | | |
| X05472cds#2_at | X05472cds#2 RNREP24R Rat 2.4 kb | 4218 | 4342 | 945 | 541 | 6 |
| | repeat DNA right terminal region | | | | | |
| X06564_at | X06564 Rat mRNA for 140-kD NCAM | 47 | 28 | 309 | 281 | 8 |
| | polypeptide/cds = 208.2784/ | | | | | |
| | gb = X06564/gi = 56736/ug = Rn.11283/ | | | | | |
| | len = 3170 | | | | | |
| X12744_at | X12744 Rat mRNA for c-erb-A thyroid | 255 | 252 | 499 | 442 | 1.9 |
| | hormone receptor/cds = 0.1198/ | | | | | |
| | gb = X12744/gi = 55931/ug = Rn.11307/ | | | | | |
| | len = 1775 | | | | | |
| X15679_at | X15679 Rat mRNA for | 377 | 403 | 120 | 201 | −1.9 |
| | preprotrypsinogen IV (EC 3.4.21.4)/ | | | | | |
| | cds = 14.757/gb = X15679/ | | | | | |
| | gi = 56813/ug = Rn.10387/len = 862 | | | | | |
| X70667cds_at | X70667cds RRMC3RA R. rattus mRNA | 221 | 249 | 426 | 508 | 2.0 |
| | for melanocortin-3 receptor | | | | | |
| AFFX_rat_5S_rRNA_at | X83747 Rattus norvegicus 5S rRNA | 348 | 357 | 154 | 146 | 2 |
| | gene (clone pRA5S2). | | | | | |

Genes that passed the filtering criteria outlined above for differential expression between 1 week extinction and 1 week withdrawal in the nucleus accumbens core. Average difference values (from GeneChip version 3.2) are listed for each gene from all groups. Affymetrix probe set numbers are listed along with the common name of the genes, if known.

TABLE 9
Core 1-Week Withdrawal to Control

| | | 1-week | 1-week | | | |
|---|---|---|---|---|---|---|
| | | withdrawal | withdrawal | 1-week | 1-week | |
| | | control | control | withdrawal | withdrawal | Fold |
| Experiment | Description | A | B | A | B | change |
| AB008424_s_at | AB008424 Rattus norvegicus mRNA for | 376 | 453 | 180 | 153 | −2.1 |
| | CYP2D3, complete cds | | | | | |
| AF069525_at | AF069525 Rattus norvegicus 190 kDa | 275 | 234 | 504 | 424 | 1.8 |
| | ankyrin isoform mRNA, complete cds/ | | | | | |
| | cds = 84.5372/gb = AF069525/ | | | | | |
| | gi = 3202045/ug = Rn.236/len = 6184 | | | | | |
| AF077354_g_at | AF077354 Rattus norvegicus ischemia | 61 | 81 | 244 | 251 | 3.5 |
| | responsive 94 kDa protein (irp94) | | | | | |
| | mRNA, complete cds | | | | | |
| AJ005425_at | AJ005425 RNAJ5425 Rattus | 86 | 22 | 373 | 394 | 1.9 |
| | norvegicus mRNA for MEF2D protein | | | | | |
| L07398_at | L07398 RATIGVCL Rattus norvegicus | 437 | 308 | 231 | 156 | −1.7 |
| | (hybridoma 56R-3) immunoglobulin | | | | | |
| | rearranged gamma-chain mRNA | | | | | |
| | variable (V) region, partial cds | | | | | |
| M80826_at | M80826 Rat intestinal trefoil protein | 334 | 322 | 112 | 102 | 3.1 |
| | mRNA, complete cds/cds = 17.262/ | | | | | |
| | gb = M80826/gi = 207446/ | | | | | |
| | ug = Rn.9960/len = 431 | | | | | |
| Rc_AI639392_at | Rat mixed-tissue library Rattus | 291 | 393 | 96 | 89 | −1.7 |
| | norvegicus cDNA clone rx02714 3′, | | | | | |
| | mRNA sequence [Rattus norvegicus] | | | | | |
| Rc_AA875131_at | rc_AA875131 UI-R-E0-bu-e-03-0-UI.s2 | 201 | 260 | 429 | 381 | 1.8 |
| | Rattus norvegicus cDNA, 3′ end/ | | | | | |
| | clone = UI-R-E0-bu-e-03-0-UI/ | | | | | |
| | clone_end = 3′/gb = AA875131/ | | | | | |
| | gi = 2980079/ug = Rn.2801/len = 575 | | | | | |
| Rc_AA899106_at | rc_AA899106 UI-R-E0-cw-d-04-0-UI.s1 | 105 | 120 | 252 | 273 | 2.3 |
| | Rattus norvegicus cDNA, 3′ end/ | | | | | |
| | clone = UI-R-E0-cw-d-04-0-UI/ | | | | | |
| | clone_end = 3′/gb = AA899106/ | | | | | |
| | gi = 3034460/ug = Rn.6031/len = 523 | | | | | |
| Rc_AI230778_at | rc_AI230778 EST227473 Rattus | 341 | 359 | 142 | 122 | −1.8 |
| | norvegicus cDNA, 3′ end/ | | | | | |
| | clone = REMDB16/clone_end = 3′/ | | | | | |
| | gb = AI230778/ug = Rn.3659/len = 560 | | | | | |
| Rc_AI230778_at | rc_AI230778 EST227473 Rattus | 359 | 341 | 122 | 142 | 2.7 |
| | norvegicus cDNA, 3′ end/ | | | | | |
| | clone = REMDB16/clone_end = 3′/ | | | | | |
| | gb = AI230778/ug = Rn.3659/len = 560 | | | | | |
| U38180_at | U38180 Rattus norvegicus reduced | 124 | 110 | 277 | 253 | 2.3 |
| | folate carrier membrane glycoprotein | | | | | |
| | mRNA, complete cds/cds = 248.1786/ | | | | | |
| | gb = U38180/gi = 1022954/ug =Rn.9042/ | | | | | |
| | len = 2410 | | | | | |
| U70268UTR#1_at | U70268UTR#1 RNMUD702 Rattus | 670 | 600 | 317 | 363 | −1.9 |
| | norvegicus mud-7 mRNA, 3′ UTR | | | | | |
| X56729mRNA_at | X56729mRNA RSCALPST Rat mRNA | 324 | 322 | 64 | 64 | 5.1 |
| | for calpastatin | | | | | |

Genes that passed the filtering criteria outlined above for differential expression between 1 week withdrawal and its corresponding control in the nucleus accumbens core. Average difference values (from GeneChip version 3.2) are listed for each gene from all groups. Affymetrix probe set numbers are listed along with the common name of the genes, if known.

TABLE 10
mPFC F Id Change 1-Week Extinction to Control

| | | 1-week | 1-week | | | |
|---|---|---|---|---|---|---|
| | | withdrawal | withdrawal | 1-week | 1-week | |
| | | control | control | withdrawal | withdrawal | Fold |
| Experiment | Description | A | B | A | B | change |
| AB002393_at | AB002393 Rattus norvegicus mRNA | 230 | 198 | −38 | −45 | −10.7 |
| | for histidase, partial cds | | | | | |
| AB012234_g_at | AB012234 Rattus norvegicus mRNA | 719 | 751 | 440 | 358 | −1.8 |
| | for NF1-X1, partial cds/cds = 0.535/ | | | | | |
| | gb = AB012234/gi = 2982735/ | | | | | |
| | ug = Rn.9647/len = 601 | | | | | |
| AF050663UTR#1_at | AF050663UTR#1 Rattus norvegicus | 492 | 471 | 200 | 173 | −2.6 |
| | activity and neurotransmitter-induced | | | | | |
| | early gene 11 (ania-11) mRNA, | | | | | |
| | 3′ UTR | | | | | |
| AF081204_s_at | AF081204 Rattus norvegicus small | 414 | 402 | 220 | 212 | −1.9 |
| | intestine sodium dependent | | | | | |
| | multivitamin transporter (SMVT) | | | | | |
| | mRNA, complete cds | | | | | |
| AF102854_at | AF102854 Rattus norvegicus | 458 | 430 | 190 | 123 | −2.2 |
| | membrane-associated guanylate | | | | | |
| | kinase-interacting protein 2 Maguin-2 | | | | | |
| | mRNA, complete cds | | | | | |
| AJ005113_g_at | AJ005113 RNAJ5113 Rattus | 447 | 469 | 232 | 258 | −1.9 |
| | norvegicus mRNA for SMC-protein | | | | | |
| | Molecular characterization of a rat | | | | | |
| | heterochromatin associated SMC- | | | | | |
| | protein | | | | | |
| AJ011115_at | AJ011115 RNO011115 Rattus | 425 | 315 | 83 | 129 | −1.9 |
| | norvegicus mRNA for endothelial nitric | | | | | |
| | oxide synthase, 5′ region, partial | | | | | |
| AJ012603UTR#1_at | AJ012603UTR#1 RNO012603 Rattus | 520 | 442 | 211 | 237 | −2.1 |
| | norvegicus mRNA for TNF-alpha | | | | | |
| | converting enzyme (TACE) | | | | | |
| D00512_g_at | D00512 RATACAL Rattus sp. mRNA | 464 | 365 | 203 | 173 | −2.1 |
| | for mitochondrial acetoacetyl-CoA | | | | | |
| | thiolase precursor, complete cds | | | | | |
| D30040_at | D30040 Rat mRNA for RAC protein | 206 | 229 | 383 | 450 | 1.9 |
| | kinase alpha, complete cds/ | | | | | |
| | cds = 42.1484/gb = D30040/gi = 485402/ | | | | | |
| | ug = Rn.11422/len = 1617 | | | | | |
| E01415cds_s_at | E01415cds cDNA encoding rat | 975 | 687 | 501 | 460 | −1.7 |
| | glutathione S transferase | | | | | |
| J02592_s_at | J02592 Rat glutathione S-transferase | 1022 | 746 | 265 | 347 | −2.9 |
| | Y-b subunit mRNA, 3′ end/cds = 0.560/ | | | | | |
| | gb = J02592/gi = 204498/ug = Rn.625/ | | | | | |
| | len = 909 | | | | | |
| J05155_at | J05155 Rat phospholipase C type IV | 228 | 222 | 72 | 88 | −2.8 |
| | mRNA, complete cds/cds = 200.3997/ | | | | | |
| | gb = J05155/gi = 206242/ug = Rn.9751/ | | | | | |
| | len = 4321 | | | | | |
| K01701_at | K01701 Rat oxytocin/neurophysin | 150 | 162 | 418 | 508 | 2.3 |
| | (Oxt) gene, complete gene, complete | | | | | |
| | cds/cds = 41.418/gb = K01701/ | | | | | |
| | gi = 205899/ug = Rn.11315/len = 530 | | | | | |
| L37971mRNA_at | L37971 mRNA RATTCRAP Rattus | 349 | 340 | 171 | 203 | −1.7 |
| | norvegicus T-cell receptor alpha-chain | | | | | |
| | mRNA | | | | | |
| L38482_at | L38482 Rattus norvegicus serine | 253 | 355 | 687 | 603 | 2.1 |
| | protease gene, complete cds/ | | | | | |
| | cds = 0.401/gb = L38482/gi = 1020080/ | | | | | |
| | ug = Rn.2427/len = 402 | | | | | |
| M22756_at | M22756 Rat 24-kDa subunit of | 1247 | 1029 | 604 | 651 | −1.8 |
| | mitochondrial NADH dehydrogenase | | | | | |
| | mRNA, 3′ end/cds = 0.725/ | | | | | |
| | gb = M22756/gi = 205627/ug = Rn.11092/ | | | | | |
| | len = 771 | | | | | |
| M25804_g_at | M25804 Rat Rev-ErbA-alpha protein | 58 | 161 | 418 | 365 | 2.0 |
| | mRNA, complete cds/cds = 501.2027/ | | | | | |
| | gb = M25804/gi = 514963/ug = Rn.10105/ | | | | | |
| | len = 2297 | | | | | |
| M27886exon_g_at | M27886exon RAT6PF2KFR Rattus | 308 | 301 | 72 | 72 | −4.2 |
| | norvegicus bifunctional enzyme 6- | | | | | |
| | phosphofructo-2-kinase/fructose | | | | | |
| | 2,6-bisphosphatase (6-PF2-K/ | | | | | |
| | Fru-2,6-P-2-ase) gene, exon 1 | | | | | |
| M31032cds#1_s_at | M31032cds#1 RATCRP01 Rat | 426 | 354 | 180 | 176 | −2.0 |
| | contiguous repeat polypeptides (CRP) | | | | | |
| | mRNA, complete cds | | | | | |
| M32061_at | M32061 Rat alpha-2B-adrenergic | 158 | 236 | 508 | 481 | 2.3 |
| | receptor (RNG-alpha-2) mRNA, | | | | | |
| | complete cds/cds = 365.1726/ | | | | | |
| | gb = M32061/gi = 202589/ug = Rn.10296/ | | | | | |
| | len = 2319 | | | | | |
| M76535cds_at | M76535cds RATCXN40A Rat gap | 734 | 746 | 353 | 278 | −2.4 |
| | junction structural protein, connexin | | | | | |
| | (CXN-40) gene, complete cds | | | | | |
| M77245_at | M77245 R. norvegicus beta′-chain | 23 | 162 | 415 | 512 | 2.3 |
| | clathrin associated protein complex | | | | | |
| | AP-1 mRNA, complete cds/cds = 39.2888/ | | | | | |
| | gb = M77245/gi = 203112/ug = Rn.9466/ | | | | | |
| | len = 3663 | | | | | |
| M77246_at | M77246 R. norvegicus beta-chain | 585 | 580 | 1166 | 1215 | 2.0 |
| | clathrin associated protein complex | | | | | |
| | AP-2 mRNA, complete cds/ | | | | | |
| | cds = 139.2994/gb = M77246/gi = 203114/ | | | | | |
| | ug = Rn.1050/len = 5402 | | | | | |
| M97662_at | M97662 Rattus norvegicus beta- | 406 | 425 | 204 | 140 | −2.1 |
| | alanine synthase mRNA, complete | | | | | |
| | cds/cds = 33.1214/gb = M97662/ | | | | | |
| | gi = 203105/ug = Rn.11110/len = 1420 | | | | | |
| rc_AI639272_at | Rat mixed-tissue library Rattus | 248 | 261 | 55 | 73 | −4.0 |
| | norvegicus cDNA clone rx03958 3′, | | | | | |
| | mRNA sequence [Rattus norvegicus] | | | | | |
| rc_AI639313_at | Rat mixed-tissue library Rattus | 576 | 564 | 191 | 154 | −3.3 |
| | norvegicus cDNA clone rx04777 3′, | | | | | |
| | mRNA sequence [Rattus norvegicus] | | | | | |
| rc_AI639195_r_at | Rat mixed-tissue library Rattus | -73 | 84 | 847 | 1043 | 4.7 |
| | norvegicus cDNA clone rx04881 3′, | | | | | |
| | mRNA sequence [Rattus norvegicus] | | | | | |
| rc_AA684641_at | rc_AA684641 EST104995 Rattus | 135 | 197 | 356 | 348 | 1.8 |
| | norvegicus cDNA, 3′ end/ | | | | | |
| | clone = RPCAE71/clone_end = 3′/ | | | | | |
| | gb = AA684641/gi = 2671239/ | | | | | |
| | ug = Rn.14675/len = 249 | | | | | |
| rc_AA799525_at | rc_AA799525 EST189022 Rattus | 682 | 583 | 371 | 370 | −1.7 |
| | norvegicus cDNA, 3′ end/ | | | | | |
| | clone =RHEAC13/clone_end = 3′/ | | | | | |
| | gb = AA799525/gi = 2862480/ | | | | | |
| | ug =Rn.1099/len = 573 | | | | | |
| rc_AA799531_g_at | rc_AA799531 EST189028 Rattus | 586 | 471 | 247 | 329 | −1.8 |
| | norvegicus cDNA, 3′ end/ | | | | | |
| | clone = RHEAC22/clone_end = 3′/ | | | | | |
| | gb = AA799531/gi = 2862486/ | | | | | |
| | ug =Rn.6198/len = 570 | | | | | |
| rc_AA818152_f_at | rc_AA818152 UI-R-A0-am-b-09-0- | 6678 | 7495 | 3932 | 4179 | −1.7 |
| | UI.s1 Rattus norvegicus cDNA, 3′ end/ | | | | | |
| | clone = UI-R-A0-am-b-09-0-UI/ | | | | | |
| | clone_end = 3′/gb = AA818152/ | | | | | |
| | gi = 2888032/ug = Rn.16465/len = 117 | | | | | |
| rc_AA818226_s_at | rc_AA818226 UI-R-A0-ah-g-06-0- | 5530 | 4813 | 2582 | 3212 | −1.8 |
| | UI.s1 Rattus norvegicus cDNA, 3′ end/ | | | | | |
| | clone = UI-R-A0-ah-g-06-0-UI/ | | | | | |
| | clone_end = 3′/gb = AA818226/ | | | | | |
| | gi = 2888106/ug = Rn.2528/len = 609 | | | | | |
| rc_AA851403_g_at | rc_AA851403 EST194171 Rattus | 1728 | 1725 | 781 | 1074 | −1.9 |
| | norvegicus cDNA, 3′ end/ | | | | | |
| | clone = RPLAG17/clone_end = 3′/ | | | | | |
| | gb = AA851403/gi = 2938943/ | | | | | |
| | ug = Rn.3383/len = 393 | | | | | |
| rc_AA851403_at | rc_AA851403 EST194171 Rattus | 291 | 296 | 131 | 127 | −2.3 |
| | norvegicus cDNA, 3′ end/ | | | | | |
| | clone = RPLAG17/clone_end = 3′/ | | | | | |
| | gb = AA851403/gi = 2938943/ | | | | | |
| | ug = Rn.3383/len = 393 | | | | | |
| rc_AA852004_s_at | rc_AA852004 EST194773 Rattus | 780 | 722 | 1393 | 1184 | 1.7 |
| | norvegicus cDNA, 3′ end/ | | | | | |
| | clone = RSPAP38/clone_end = 3′/ | | | | | |
| | gb = AA852004/gi = 2939544/ | | | | | |
| | ug = Rn.2204/len = 368 | | | | | |
| rc_AA859299_at | rc_AA859299 UI-R-E0-cj-b-02-0-UI.s1 | 309 | 302 | 721 | 553 | 2.1 |
| | Rattus norvegicus cDNA, 3′ end/ | | | | | |
| | clone = UI-R-E0-cj-b-02-0-UI/ | | | | | |
| | clone_end = 3′/gb = AA859299/ | | | | | |
| | gi = 2948650/ug = Rn.9517/len = 529 | | | | | |
| rc_AA859837_g_at | rc_AA859837 UI-R-E0-cc-g-09-0- | 3301 | 2600 | 1719 | 1534 | −1.8 |
| | UI.s1 Rattus norvegicus cDNA, 3′ end/ | | | | | |
| | clone = UI-R-E0-cc-g-09-0-UI/ | | | | | |
| | clone_end = 3′/gb = AA859837/ | | | | | |
| | gi = 2949357/ug = Rn.24783/len = 486 | | | | | |
| rc_AA859922_at | rc_AA859922 UI-R-E0-cg-c-04-0- | 657 | 601 | 253 | 321 | −2.2 |
| | UI.s1 Rattus norvegicus cDNA, 3′ end/ | | | | | |
| | clone = UI-R-E0-cg-c-04-0-UI/ | | | | | |
| | clone_end = 3′/gb = AA859922/ | | | | | |
| | gi = 2949442/ug = Rn.819/len = 373 | | | | | |
| rc_AA866477_at | rc_AA866477 UI-R-E-br-h-03-0-UI.s1 | 1136 | 1364 | 601 | 702 | −1.9 |
| | Rattus norvegicus cDNA, 3′ end/ | | | | | |
| | clone = UI-R-E0-br-h-03-0-UI/ | | | | | |
| | clone_end = 3′/gb = AA866477/ | | | | | |
| | gi = 2961938/ug = Rn.2026/len = 488 | | | | | |
| rc_AA875420_at | rc_AA875420 UI-R-E0-cs-e-08-0-UI.s1 | 291 | 339 | 20 | 47 | −9.4 |
| | Rattus norvegicus cDNA, 3′ end/ | | | | | |
| | clone = UI-R-E0-cs-e-08-0-UI/ | | | | | |
| | clone_end = 3′/gb = AA875420/ | | | | | |
| | gi = 2980368/ug = Rn.21413/len = 499 | | | | | |
| rc_AA892006_at | rc_AA892006 EST195809 Rattus | −157 | −160 | 510 | 449 | 23.96 |
| | norvegicus cDNA, 3′ end/ | | | | | |
| | clone = RKIAK60/clone_end = 3′/ | | | | | |
| | gb = AA892006/gi = 3018885/ | | | | | |
| | ug = Rn.11519/len = 443 | | | | | |
| rc_AA892800_at | rc_AA892800 EST196603 Rattus | −203 | −165 | 390 | 313 | 1.8 |
| | norvegicus cDNA, 3′ end/ | | | | | |
| | clone = RKIAX43/clone_end = 3′/ | | | | | |
| | gb = AA892800/gi = 3019679/ | | | | | |
| | ug = Rn.3609/len = 493 | | | | | |
| rc_AA894296_at | rc_AA894296 EST198099 Rattus | 222 | 252 | 457 | 573 | 2.2 |
| | norvegicus cDNA, 3′ end/ | | | | | |
| | clone = RSPAW17/clone_end = 3′/ | | | | | |
| | gb = AA894296/gi = 3021175/ | | | | | |
| | ug = Rn.3760/len = 600 | | | | | |
| rc_AA899106_at | rc_AA899106 UI-R-E0-cw-d-04-0-UI.s1 | 482 | 459 | 292 | 214 | −1.9 |
| | Rattus norvegicus cDNA, 3′ end/ | | | | | |
| | clone = UI-R-E0-cw-d-04-0-UI/ | | | | | |
| | clone_end = 3′/gb = AA899106/ | | | | | |
| | gi = 3034460/ug = Rn.6031/len = 523 | | | | | |
| rc_AA899253_at | rc_AA899253 UI-R-E0-cz-g-07-0-UI.s1 | 832 | 904 | 401 | 537 | −1.9 |
| | Rattus norvegicus cDNA, 3′ end/ | | | | | |
| | clone = UI-R-E0-cz-g-07-0-UI/ | | | | | |
| | clone_end = 3′/gb = AA899253/ | | | | | |
| | gi = 3034607/ug = Rn.9560/len = 410 | | | | | |
| rc_AA945152_s_at | rc_AA945152 EST200651 Rattus | 22042 | 30447 | 12827 | 15228 | −1.9 |
| | norvegicus cDNA, 3′ end/ | | | | | |
| | clone = RLIAH24/clone_end = 3′/ | | | | | |
| | gb = AA945152/ug = Rn.4241/len = 777 | | | | | |
| rc_AI009191_at | rc_AI009191 EST203642 Rattus | 441 | 615 | 981 | 821 | 1.7 |
| | norvegicus cDNA, 3′ end/ | | | | | |
| | clone = REMBK67/clone_end = 3′/ | | | | | |
| | gb = AI009191/ug = Rn.2432/len = 484 | | | | | |
| rc_AI058941_s_at | rc_AI058941 UI-R-C1-Ir-b-07-0-UI.s1 | 570 | 562 | 214 | 252 | −2.4 |
| | Rattus norvegicus cDNA, 3′ end/ | | | | | |
| | clone = UI-R-C1-Ir-b-07-0-UI/ | | | | | |
| | clone_end = 3′/gb = AI058941/ | | | | | |
| | ug = Rn.4231/len = 476 | | | | | |
| rc_AI072770_s_at | rc_AI072770 UI-R-Y0-md-g-02-0-UI.s1 | 330 | 258 | 462 | 566 | 1.7 |
| | Rattus norvegicus cDNA, 3′ end/ | | | | | |
| | clone = UI-R-Y0-md-g-02-0-UI/ | | | | | |
| | clone_end = 3′/gb = AI072770/ | | | | | |
| | ug = Rn.4550/len = 333 | | | | | |
| rc_AI103396_g_at | rc_AI103396 EST212685 Rattus | 26045 | 26104 | 16586 | 12308 | −1.8 |
| | norvegicus cDNA, 3′ end/ | | | | | |
| | clone = REMCB47/clone_end = 3′/ | | | | | |
| | gb = AI103396/gi = 3707945/ | | | | | |
| | ug = Rn.221/len = 443 | | | | | |
| rc_AI137043_at | rc_AI137043 UI-R-C2p-oj-c-01-0-UI.s1 | 371 | 442 | 95 | 35 | −2.0 |
| | Rattus norvegicus cDNA, 3′ end/ | | | | | |
| | clone = UI-R-C2p-oj-c-01-0-UI/ | | | | | |
| | clone_end = 3′/gb = AI137043/ | | | | | |
| | ug = Rn.22168/len = 436 | | | | | |
| rc_AI137856_s_at | rc_AI137856 UI-R-C0-ik-a-10-0-UI.s1 | 510 | 469 | 212 | 219 | −2.3 |
| | Rattus norvegicus cDNA, 3′ end/ | | | | | |
| | clone = UI-R-C0-ik-a-10-0-UI/ | | | | | |
| | clone_end = 3′/gb = AI137856/ | | | | | |
| | ug = Rn.11359/len = 384 | | | | | |
| rc_AI176307_at | rc_AI176307 EST219889 Rattus | 1777 | 2176 | 952 | 906 | −2.1 |
| | norvegicus cDNA, 3′ end/ | | | | | |
| | clone = ROVBP82/clone_end = 3′/ | | | | | |
| | gb = AI176307/ug = Rn.10427/len = 678 | | | | | |
| rc_AI176621_at | rc_AI176621 EST220210 Rattus | 400 | 289 | 196 | 117 | −1.7 |
| | norvegicus cDNA, 3′ end/ | | | | | |
| | clone = ROVBU65/clone_end = 3′/ | | | | | |
| | gb = AI176621/ug = Rn.1979/len = 620 | | | | | |
| rc_AI177503_at | rc_AI177503 EST221135 Rattus | 276 | 273 | 520 | 454 | 1.8 |
| | norvegicus cDNA, 3′ end/ | | | | | |
| | clone = RPLCA81/clone_end = 3′/ | | | | | |
| | gb = AI177503/ug = Rn.11066/len = 575 | | | | | |
| rc_AI232012_at | rc_AI232012 EST228700 Rattus | 1062 | 957 | 575 | 590 | −1.7 |
| | norvegicus cDNA, 3′ end/ | | | | | |
| | clone = RHECR46/clone_end = 3′/ | | | | | |
| | gb = AI232012/ug = Rn.1128/len = 586 | | | | | |
| rc_AI232321_at | rc_AI232321 EST229009 Rattus | 312 | 333 | 177 | 173 | −1.8 |
| | norvegicus cDNA, 3′ end/ | | | | | |
| | clone =RKICA22/clone_end = 3′/ | | | | | |
| | gb = AI232321/ug = Rn.24630/len = 590 | | | | | |
| rc_AI234060_s_at | rc_AI234060 EST230748 Rattus | 119 | 111 | 322 | 302 | 2.71 |
| | norvegicus cDNA, 3′ end/ | | | | | |
| | clone = RLUCU63/clone_end = 3′/ | | | | | |
| | gb = AI234060/ug = Rn.11372/len = 363 | | | | | |
| S74801_s_at | S74801 H(+)-K(+)-ATPase alpha- | 238 | 239 | 101 | 78 | −2.7 |
| | subunit [rats, Sprague-Dawley, | | | | | |
| | kidney, mRNA Partial, 1361 nt] | | | | | |
| U16025_at | U16025 Rattus norvegicus class Ib | 470 | 442 | 267 | 211 | −1.9 |
| | RT1 mRNA, complete cds/cds = 0.1019/ | | | | | |
| | gb = U16025/gi = 717092/ug = Rn.19044/ | | | | | |
| | len = 1311 | | | | | |
| U23769_at | U23769 Rattus norvegicus CLP36 | 172 | 162 | 285 | 284 | 1.7 |
| | (clp36) mRNA, complete cds/ | | | | | |
| | cds = 66.1049/gb = U23769/gi = 1020150/ | | | | | |
| | ug = Rn.11170/len = 1392 | | | | | |
| U32575_g_at | U32575 RNU32575 Rattus norvegicus | 364 | 360 | 32 | 38 | −1.8 |
| | (rsec6) mRNA, complete cds | | | | | |
| U56261_s_at | U56261 RNU56261 Rattus norvegicus | 122 | 144 | 300 | 303 | 2.27 |
| | SNAP-25a mRNA, partial cds | | | | | |
| U70270UTR#1_f_at | U70270UTR#1 RNMUD402 Rattus | 550 | 516 | 340 | 270 | −1.7 |
| | norvegicus mud-4 mRNA, 3′ UTR | | | | | |
| U72995_at | U72995 Rattus norvegicus Rab3 | 273 | 248 | 579 | 497 | 2.1 |
| | GDP/GTP exchange protein mRNA, | | | | | |
| | complete cds/cds = 191.4999/ | | | | | |
| | gb = U72995/gi =1947049/ug = Rn.9786/ | | | | | |
| | len = 5249 | | | | | |
| U89745_at | U89745 Rattus norvegicus unknown | 1075 | 1106 | 654 | 587 | −1.8 |
| | protein mRNA, partial cds/cds = 0.293/ | | | | | |
| | gb = U89745/gi = 1895082/ug = Rn.10720/ | | | | | |
| | len = 1114 | | | | | |
| X53581cds#5_f_at | X53581cds#5 RNLINED R. norvegicus | 1225 | 1155 | 2071 | 2773 | 2.0 |
| | long interspersed repetitive DNA | | | | | |
| | containing 7 ORF's | | | | | |
| X69903_at | X69903 R. norvegicus mRNA for | 491 | 377 | 118 | 146 | −2.2 |
| | interleukin 4 receptor/cds = 9.2411/ | | | | | |
| | gb = X69903/gi = 56390/ug = Rn.10471/ | | | | | |
| | len = 2450 | | | | | |
| Y17048_g_at | Y17048 RNCALDE Rattus norvegicus | 492 | 465 | 912 | 916 | 1.91 |
| | mRNA for caldendrin | | | | | |
| Z50052_at | Z50052 R. norvegicus mRNA for C4BP | 220 | 226 | 45 | 61 | −4.2 |
| | beta chain protein/cds = 265.1041/ | | | | | |
| | gb = Z50052/gi = 899381/ug = Rn.11151/ | | | | | |
| | len = 1091 | | | | | |

Genes that passed the filtering criteria outlined above for differential expression between 1 week extinction and its corresponding control in the medial prefrontal cortex (mPFC). Average difference values (from GeneChip version 3.2) are listed for each gene from all groups. Affymetrix probe set numbers are listed along with the common name of the genes, if known.

TABLE 11
mPFC Fold Change 1-Week Extinction to Withdrawal

| | | 1-week | 1-week | 1-week | 1-week | |
|---|---|---|---|---|---|---|
| | | withdrawal | withdrawal | extinction | extinction | Fold |
| Experiment | Description | B | A | B | A | change |
| AB004559_at | AB004559 Rattus norvegicus mRNA for | 34 | 109 | 393 | 431 | −2.1 |
| | multispecific organic anion transporter, | | | | | |
| | complete cds/cds = 275.1930/ | | | | | |
| | gb = AB004559/gi = 2361034/ | | | | | |
| | ug = Rn.11113/len = 2221 | | | | | |
| AF020618_g_at | AF020618 Rattus norvegicus | 371 | 416 | 205 | 139 | 1.9 |
| | progression elevated gene 3 protein | | | | | |
| | mRNA, complete cds | | | | | |
| AF044201_at | AF044201 Rattus norvegicus neural | 1152 | 974 | 636 | 546 | 1.8 |
| | membrane protein 35 mRNA, complete | | | | | |
| | cds | | | | | |
| AF051526_at | AF051526 Rattus norvegicus class A | 249 | 242 | 98 | 119 | −2.3 |
| | calcium channel variant riA-I (BCCA1) | | | | | |
| | mRNA, partial cds/cds = 0.2375/ | | | | | |
| | gb = AF051526/gi = 2961609/ | | | | | |
| | ug = Rn.11281/len = 2427 | | | | | |
| AF076183_at | AF076183 Rattus norvegicus cytosolic | 405 | 319 | 215 | 177 | 1.7 |
| | sorting protein PACS-1a (PACS-1) | | | | | |
| | mRNA, complete cds | | | | | |
| AF091566_f_at | AF091566 Rattus norvegicus isolate | 303 | 407 | 9 | −44 | 1.8 |
| | HTF-SP1 olfactory receptor mRNA, | | | | | |
| | partial cds | | | | | |
| AF102854_at | AF102854 Rattus norvegicus | 379 | 395 | 123 | 190 | 1.9 |
| | membrane-associated guanylate | | | | | |
| | kinase-interacting protein 2 Maguin-2 | | | | | |
| | mRNA, complete cds | | | | | |
| AFFX_Rat_beta- | V01217 Rat gene encoding cytoplasmic | 1708 | 2336 | 992 | 1100 | 1.9 |
| actin_5_at | beta-actin (_5, _M, _3 represent | | | | | |
| | transcript regions 5 prime, Middle, and | | | | | |
| | 3 prime respectively) | | | | | |
| AJ005113_at | AJ005113 RNAJ5113 Rattus | 299 | 384 | 103 | 195 | 1.7 |
| | norvegicus mRNA for SMC-protein | | | | | |
| | Molecular characterization of a rat | | | | | |
| | heterochromatin associated SMC- | | | | | |
| | protein | | | | | |
| AJ005394_at | AJ005394 RNJ005394 Rattus | 355 | 309 | 103 | 96 | −3.3 |
| | norvegicus mRNA for collagen alpha 1 | | | | | |
| | type V | | | | | |
| AJ011005_at | AJ011005 RNO011005 Rattus | 848 | 988 | 460 | 359 | 2.2 |
| | norvegicus mRNA for Ptx3 protein | | | | | |
| D00512_g_at | D00512 RATACAL Rattus sp. mRNA | 386 | 470 | 173 | 203 | 2.1 |
| | for mitochondrial acetoacetyl-CoA | | | | | |
| | thiolase precursor, complete cds | | | | | |
| D10757_at | D10757 RATPRORR12 Rat mRNA for | 444 | 484 | 230 | 246 | −2.0 |
| | proteasome subunit R-RING12, | | | | | |
| | complete cds | | | | | |
| D13212_s_at | D13212 RATNMDARC Rat mRNA for | 484 | 499 | 251 | 280 | −1.9 |
| | N-methyl-D-aspartate receptor subunit | | | | | |
| | (NMDAR2C) | | | | | |
| D14819_g_at | D14819 RATCBPP23B Rat mRNA for | 679 | 890 | 484 | 392 | 1.8 |
| | calcium-binding protein P23k beta, | | | | | |
| | partial cds | | | | | |
| D30734_at | D30734 RATGAP1M Rat mRNA for | 353 | 383 | 220 | 208 | 1.7 |
| | Ras GTPase-activating protein, | | | | | |
| | complete cds | | | | | |
| J02669_s_at | J02669 Rat cytochrome P-450a (3- | 858 | 1000 | 539 | 530 | 1.7 |
| | methylchlanthrene-inducible; with high | | | | | |
| | testosterone 7-alpha activity), mRNA, | | | | | |
| | complete cds/cds = 19.1497/gb = J02669/ | | | | | |
| | gi = 203766/ug = Rn.10904/len = 1687 | | | | | |
| J05499_at | J05499 Rattus norvegicus L-glutamine | 216 | 215 | 114 | 128 | −1.8 |
| | amidohydrolase mRNA, complete cds/ | | | | | |
| | cds = 131.1738/gb = J05499/gi = 1196813/ | | | | | |
| | ug = Rn.10202/len = 2225 | | | | | |
| K01701_at | K01701 Rat oxytocin/neurophysin (Oxt) | 150 | 131 | 508 | 418 | −2.3 |
| | gene, complete gene, complete cds/ | | | | | |
| | cds = 41.418/gb = K01701/gi = 205899/ | | | | | |
| | ug = Rn.11315/len = 530 | | | | | |
| L07398_at | L07398 RATIGVCL Rattus norvegicus | 189 | 148 | 449 | 527 | −2.4 |
| | (hybridoma 56R-3) immunoglobulin | | | | | |
| | rearranged gamma-chain mRNA | | | | | |
| | variable (V) region, partial cds | | | | | |
| L38482_at | L38482 Rattus norvegicus serine | 290 | 360 | 603 | 687 | −2.0 |
| | protease gene, complete cds/cds = 0.401/ | | | | | |
| | gb = L38482/gi = 1020080/ug = Rn.2427/ | | | | | |
| | len = 402 | | | | | |
| M11071_f_at | M11071 Rat MHC class I cell surface | 3217 | 3367 | 792 | 959 | −3.8 |
| | antigen mRNA/cds = 0.330/gb = M11071/ | | | | | |
| | gi = 205414/ug = Rn.11168/len = 824 | | | | | |
| M20721_f_at | M20721 RATPRPA Rat proline-rich | 282 | 281 | 129 | 128 | −2.2 |
| | protein (PRP-1) mRNA, partial cds | | | | | |
| M25804_g_at | M25804 Rat Rev-ErbA-alpha protein | 175 | 138 | 365 | 418 | −2.0 |
| | mRNA, complete cds/cds = 501.2027/ | | | | | |
| | gb = M25804/gi = 514963/ug = Rn.10105/ | | | | | |
| | len = 2297 | | | | | |
| M27886exon_g_at | M27886exon RAT6PF2KFR Rattus | 223 | 215 | 72 | 72 | −3.0 |
| | norvegicus bifunctional enzyme | | | | | |
| | 6-phosphofructo-2-kinase/fructose | | | | | |
| | 2,6-bisphosphatase (6-PF2-K/Fru-2,6- | | | | | |
| | P-2-ase) gene, exon 1 | | | | | |
| M31018_f_at | M31018 Rattus norvegicus MHC class I | 474 | 454 | 232 | 156 | 2.1 |
| | RT1.Aa alpha-chain precursor mRNA, | | | | | |
| | complete cds/cds = 9.1124/gb = M31018/ | | | | | |
| | gi = 1877415/ug = Rn.3577/len = 1590 | | | | | |
| M77809_at | M77809 Rat betaglycan mRNA, | 378 | 339 | 117 | 120 | −3.0 |
| | complete cds/cds = 334.2895/gb = M77809/ | | | | | |
| | gi = 203137/ug = Rn.9953/len = 3931 | | | | | |
| Rc_AA799467_at | rc_AA799467 EST188964 Rattus | 413 | 486 | 292 | 218 | 1.8 |
| | norvegicus cDNA, 3′ end/ | | | | | |
| | clone = RHEAB38/clone_end = 3′/ | | | | | |
| | gb = AA799467/gi = 2862422/ug = Rn.4036/ | | | | | |
| | len = 568 | | | | | |
| Rc_AA799792_at | rc_AA799792 EST189289 Rattus | 101 | 92 | 261 | 291 | 2.9 |
| | norvegicus cDNA, 3′ end/clone = RHEAF41/ | | | | | |
| | clone_end = 3′/gb = AA799792/ | | | | | |
| | gi = 2862747/ug = Rn.7461/len = 615 | | | | | |
| Rc_AA799964_at | rc_AA799964 EST189461 Rattus | 17 | 3 | 309 | 270 | 14.5 |
| | norvegicus cDNA, 3′ end/clone = RHEAH66/ | | | | | |
| | clone_end = 3′/gb = AA799964/ | | | | | |
| | gi = 2862919/ug = Rn.6261/len = 452 | | | | | |
| Rc_AA800005_at | rc_AA800005 EST189502 Rattus | 328 | 315 | 701 | 636 | 2.1 |
| | norvegicus cDNA, 3′ end/clone = RHEAI20/ | | | | | |
| | clone_end = 3′/gb = AA800005/ | | | | | |
| | gi = 2862960/ug = Rn.1465/len = 628 | | | | | |
| Rc_AA800250_at | rc_AA800250 EST189747 Rattus | 708 | 567 | 912 | 1264 | −1.7 |
| | norvegicus cDNA, 3′ end/clone = RHEAM94/ | | | | | |
| | clone_end = 3′/gb = AA800250/ | | | | | |
| | gi = 2863205/ug = Rn.3593/len = 666 | | | | | |
| Rc_AA800604_g_at | rc_AA800604 EST190101 Rattus | 413 | 396 | 159 | −18 | 2.0 |
| | norvegicus cDNA, 3′ end/clone = RLUAB65/ | | | | | |
| | clone_end = 3′/gb = AA800604/ | | | | | |
| | gi = 2863559/ug = Rn.8590/len = 579 | | | | | |
| Rc_AA800737_at | rc_AA800737 EST190234 Rattus | 219 | 206 | 430 | 322 | −1.8 |
| | norvegicus cDNA, 3′ end/clone = RLUAK84/ | | | | | |
| | clone_end = 3′/gb = AA800737/ | | | | | |
| | gi = 2863692/ug = Rn.6628/len = 626 | | | | | |
| Rc_AA851403_at | rc_AA851403 EST194171 Rattus | 309 | 328 | 131 | 127 | −2.5 |
| | norvegicus cDNA, 3′ end/clone = RPLAG17/ | | | | | |
| | clone_end = 3′/gb = AA851403/ | | | | | |
| | gi = 2938943/ug = Rn.3383/len = 393 | | | | | |
| Rc_AA859585_at | rc_AA859585 UI-R-E0-bv-d-05-0-UI.s1 | 471 | 544 | 176 | 262 | 2.2 |
| | Rattus norvegicus cDNA, 3′ end/ | | | | | |
| | clone = UI-R-E0-bv-d-05-0-UI/ | | | | | |
| | clone_end = 3′/gb = AA859585/ | | | | | |
| | gi = 2949105/ug = Rn.24950/len = 516 | | | | | |
| Rc_AA859722_at | rc_AA859722 UI-R-E0-bx-h-09-0-UI.s1 | 459 | 381 | −1 | 5 | −21.0 |
| | Rattus norvegicus cDNA, 3′ end/ | | | | | |
| | clone = UI-R-E0-bx-h-09-0-UI/ | | | | | |
| | clone_end = 3′/gb = AA859722/ | | | | | |
| | gi = 2949242/ug = Rn.70/len = 460 | | | | | |
| Rc_AA859922_at | rc_AA859922 UI-R-E0-cg-c-04-0-UI.s1 | 615 | 712 | 321 | 253 | 2.3 |
| | Rattus norvegicus cDNA, 3′ end/ | | | | | |
| | clone = UI-R-E0-cg-c-04-0-UI/ | | | | | |
| | clone_end = 3′/gb = AA859922/ | | | | | |
| | gi = 2949442/ug = Rn.819/len = 373 | | | | | |
| Rc_AA874919_at | rc_AA874919 UI-R-E0-ck-g-09-0-UI.s1 | 224 | 221 | 365 | 428 | −1.8 |
| | Rattus norvegicus cDNA, 3′ end/ | | | | | |
| | clone = UI-R-E0-ck-g-09-0-UI/ | | | | | |
| | clone_end = 3′/gb = AA874919/ | | | | | |
| | gi = 2979867/ug = Rn.3174/len = 542 | | | | | |
| Rc_AA875411_s_at | rc_AA875411 UI-R-E0-cs-b-11-0-UI.s1 | 115 | 191 | 425 | 422 | −2.1 |
| | Rattus norvegicus cDNA, 3′ end/ | | | | | |
| | clone = UI-R-E0-cs-b-11-0-UI/ | | | | | |
| | clone_end = 3′/gb = AA875411/ | | | | | |
| | gi = 2980359/ug = Rn.2911/len = 423 | | | | | |
| Rc_AA892006_at | rc_AA892006 EST195809 Rattus | −59 | −76 | 510 | 449 | 24.0 |
| | norvegicus cDNA, 3′ end/ | | | | | |
| | clone = RKIAK60/clone_end = 3′/ | | | | | |
| | gb = AA892006/gi = 3018885/ | | | | | |
| | ug =Rn.11519/len = 443 | | | | | |
| Rc_AA892179_at | rc_AA892179 EST195982 Rattus | 210 | 198 | 421 | 358 | −1.9 |
| | norvegicus cDNA, 3′ end/ | | | | | |
| | clone = RKIAN31/clone_end = 3′/ | | | | | |
| | gb = AA892179/gi = 3019058/ | | | | | |
| | ug = Rn.9031/len = 428 | | | | | |
| Rc_AA892800_at | rc_AA892800 EST196603 Rattus | 35 | −350 | 313 | 390 | −1.8 |
| | norvegicus cDNA, 3′ end/clone = RKIAX43/ | | | | | |
| | clone_end = 3′/gb = AA892800/ | | | | | |
| | gi = 3019679/ug = Rn.3609/len = 493 | | | | | |
| Rc_AA892801_g_at | rc_AA892801 EST196604 Rattus | 497 | 658 | 277 | 354 | 1.8 |
| | norvegicus cDNA, 3′ end/clone = RKIAX44/ | | | | | |
| | clone_end = 3′/gb = AA892801/ | | | | | |
| | gi = 3019680/ug = Rn.3610/len = 528 | | | | | |
| Rc_AA892828_at | rc_AA892828 EST196631 Rattus | 343 | 240 | 444 | 551 | −1.7 |
| | norvegicus cDNA, 3′ end/clone = RKIAX75/ | | | | | |
| | clone_end = 3′/gb = AA892828/ | | | | | |
| | gi = 3019707/ug = Rn.2273/len = 626 | | | | | |
| Rc_AA893210_at | rc_AA893210 EST197013 Rattus | −20 | 28 | 329 | 361 | 17.3 |
| | norvegicus cDNA, 3′ end/clone = RKIBD55/ | | | | | |
| | clone_end = 3′/gb = AA893210/ | | | | | |
| | gi = 3020089/ug = Rn.11141/len = 608 | | | | | |
| Rc_AI009191_at | rc_AI009191 EST203642 Rattus | 512 | 542 | 821 | 981 | −1.7 |
| | norvegicus cDNA, 3′ end/clone = REMBK67/ | | | | | |
| | clone_end = 3′/gb = AI009191/ | | | | | |
| | ug = Rn.2432/len = 484 | | | | | |
| Rc_AI013993_at | rc_AI013993 EST207548 Rattus | 279 | 248 | 100 | 102 | −2.6 |
| | norvegicus cDNA, 3′ end/clone = RSPBC95/ | | | | | |
| | clone_end = 3′/gb = AI013993/ | | | | | |
| | ug = Rn.221/len = 514 | | | | | |
| Rc_AI014094_g_at | rc_AI014094 EST207649 Rattus | 374 | 335 | 195 | 187 | 1.8 |
| | norvegicus cDNA, 3′ end/clone = RSPBE87/ | | | | | |
| | clone_end = 3′/gb = AI014094/ | | | | | |
| | ug = Rn.221/len = 569 | | | | | |
| Rc_AI101320_at | rc_AI101320 EST210609 Rattus | 368 | 341 | 119 | 125 | −2.9 |
| | norvegicus cDNA, 3′ end/clone = RBRBL38/ | | | | | |
| | clone_end = 3′/gb = AI101320/ | | | | | |
| | ug = Rn.22459/len = 616 | | | | | |
| Rc_AI137856_s_at | rc_AI137856 UI-R-C0-ik-a-10-0-UI.s1 | 394 | 392 | 212 | 219 | −1.8 |
| | Rattus norvegicus cDNA, 3′ end/ | | | | | |
| | clone = UI-R-C0-ik-a-10-O-UI/ | | | | | |
| | clone_end = 3′/gb = AI137856/ | | | | | |
| | ug = Rn.11359/len = 384 | | | | | |
| Rc_AI172097_g_at | rc_AI172097 EST218092 Rattus | 533 | 441 | 123 | 200 | 2.4 |
| | norvegicus cDNA, 3′ end/clone = RMUBU88/ | | | | | |
| | clone_end = 3′/gb = AI172097/ | | | | | |
| | gi = 3712137/ug = Rn.20418/len = 570 | | | | | |
| Rc_AI176307_at | rc_AI176307 EST219889 Rattus | 1840 | 1824 | 906 | 952 | −2.0 |
| | norvegicus cDNA, 3′ end/clone = ROVBP82/ | | | | | |
| | clone_end = 3′/gb = AI176307/ | | | | | |
| | ug = Rn.10427/len = 678 | | | | | |
| Rc_AI231213_g_at | rc_AI231213 EST227901 Rattus | 70 | 45 | 213 | 216 | 3.7 |
| | norvegicus cDNA, 3′ end/clone = REMDH23/ | | | | | |
| | clone_end = 3′/gb = AI231213/ | | | | | |
| | ug = Rn.3022/len = 582 | | | | | |
| Rc_AI231472_s_at | rc_AI231472 EST228160 Rattus | 160 | 171 | 384 | 349 | 2.2 |
| | norvegicus cDNA, 3′ end/clone = REMDK57/ | | | | | |
| | clone_end = 3′/gb = AI231472/ | | | | | |
| | ug = Rn.2953/len = 549 | | | | | |
| Rc_AI639197_at | Rat mixed-tissue library Rattus | 706 | 904 | 379 | 388 | 2.1 |
| | norvegicus cDNA clone rx02020 3′, | | | | | |
| | mRNA sequence [Rattus norvegicus] | | | | | |
| Rc_AI639236_at | Rat mixed-tissue library Rattus | 642 | 653 | 232 | 280 | −2.5 |
| | norvegicus cDNA clone rz00757 3′, | | | | | |
| | mRNA sequence [Rattus norvegicus] | | | | | |
| Rc_AI639313_at | Rat mixed-tissue library Rattus | 581 | 667 | 191 | 154 | 3.1 |
| | norvegicus cDNA clone rx04777 3′, | | | | | |
| | mRNA sequence [Rattus norvegicus] | | | | | |
| Rc_H31420_at | rc_H31420 EST105436 Rattus | 649 | 751 | 1229 | 1569 | −2.0 |
| | norvegicus cDNA, 3′ end/clone = RPCAJ34/ | | | | | |
| | clone_end = 3′/gb = H31420/ | | | | | |
| | gi = 976837/ug = Rn.8443/len = 312 | | | | | |
| S54212_at | S54212 ciliary neurotrophic factor | 302 | 414 | 205 | 209 | 1.7 |
| | receptor alpha component [rats, brain, | | | | | |
| | mRNA, 1332 nt] | | | | | |
| U20283_at | U20283 Rattus norvegicus syntaxin | 206 | 149 | 456 | 442 | −2.2 |
| | binding protein Munc18-2 mRNA, | | | | | |
| | complete cds/cds = 6.1790/gb = U20283/ | | | | | |
| | gi = 1022680/ug = Rn.10121/len = 2118 | | | | | |
| U35774_at | U35774 Rattus norvegicus cytosolic | 524 | 396 | 245 | 284 | 1.7 |
| | branch chain aminotransferase mRNA, | | | | | |
| | complete cds/cds = 62.1297/gb = U35774/ | | | | | |
| | gi = 1173633/ug = Rn.8273/len = 1370 | | | | | |
| U36773_at | U36773 RNU36773 Rattus norvegicus | 134 | 143 | 411 | 549 | −2.4 |
| | glycerol-3-phosphate acyltransferase | | | | | |
| | mRNA, nuclear gene encoding | | | | | |
| | mitochondrial protein, partial cds | | | | | |
| U37101_at | U37101 RRU37101 Rattus rattus | 436 | 403 | 59 | 179 | 2.1 |
| | granulocyte colony stimulating factor | | | | | |
| | mRNA, complete cds | | | | | |
| U50185_g_at | U50185 RNU50185 Rattus norvegicus | 345 | 446 | 229 | 197 | 1.8 |
| | kidney protein phosphatase 1 myosin | | | | | |
| | binding subunit mRNA, partial cds | | | | | |
| U84402_at | U84402 RNU84402 Rattus norvegicus | 537 | 611 | 256 | 219 | 2.4 |
| | smoothened mRNA, complete cds | | | | | |
| U92284_at | U92284 Rattus norvegicus GABA-A | 216 | 210 | 78 | 72 | −2.9 |
| | receptor epsilon subunit gene, partial | | | | | |
| | cds/cds = 0.1154/gb = U92284/ | | | | | |
| | gi = 2735328/ug = Rn.10869/len = 1600 | | | | | |
| X14848cds#12_at | X14848cds#12 MIRNXX Rattus | 461 | 379 | 218 | 221 | 1.9 |
| | norvegicus mitochondrial genome | | | | | |
| X56325mRNA_s_at | X56325mRNA RN2A1GL R. norvegicus | 2029 | 1486 | 1069 | 976 | 1.7 |
| | 2-alpha-1 globin gene | | | | | |
| X58294_at | X58294 R. norvegicus mRNA for | 88 | 254 | 387 | 426 | −1.8 |
| | carbonic anhydrase II/cds = 8.790/ | | | | | |
| | gb = X58294/gi = 55837/ug = Rn.3525/ | | | | | |
| | len = 1459 | | | | | |
| X62086mRNA_s_at | X62086 mRNA RNCYP3A1 | 236 | 254 | 433 | 599 | −2.1 |
| | R. norvegicus CYP3A1 gene for | | | | | |
| | cytochrome P450 PCN1 | | | | | |
| X69903_at | X69903 R. norvegicus mRNA for | 417 | 408 | 146 | 118 | −3.1 |
| | interleukin 4 receptor/cds = 9.2411/ | | | | | |
| | gb = X69903/gi = 56390/ug = Rn.10471/ | | | | | |
| | len = 2450 | | | | | |
| X89968_g_at | X89968 RNSNAPGEN Rattus | 471 | 555 | 929 | 1080 | −2.0 |
| | norvegicus mRNA for alpha-soluble | | | | | |
| | NSF attachment protein | | | | | |

Genes that passed the filtering criteria outlined above for differential expression between 1 week extinction and its corresponding control in the medial prefrontal cortex (mPFC). Average difference values (from GeneChip version 3.2) are listed for each gene from all groups. Affymetrix probe set numbers are listed along with the common name of the genes, if known.

TABLE 12
mPFC Fold Change 1-Week Withdrawal to Control

| | | 1-week | 1-week | 1-week | 1-week | |
|---|---|---|---|---|---|---|
| | | withdrawal | withdrawal | withdrawal | withdrawal | Fold |
| Experiment | Description | control A | control B | A | B | change |
| AB006450_at | AB006450 Rattus norvegicus mRNA for | 167 | 198 | 409 | 326 | 1.8 |
| | Tim17, complete cds/cds = 4,519/ | | | | | |
| | gb = AB006450/gi = 2335036/ | | | | | |
| | ug = Rn.2099/len = 944 | | | | | |
| AB020504_at | AB020504 Rattus norvegicus mRNA for | 181 | 202 | 385 | 361 | 2.0 |
| | PMF31, complete cds | | | | | |
| AF001898_at | AF001898 Rattus norvegicus aldehyde | 796 | 993 | 518 | 453 | −1.8 |
| | dehydrogenase (ALDH) mRNA, | | | | | |
| | complete cds/cds = 28,1533/ | | | | | |
| | gb = AF001898/gi = 2183216/ | | | | | |
| | ug = Rn.6132/len = 2095 | | | | | |
| AF091566_f_at | AF091566 Rattus norvegicus isolate | −151 | −48 | 407 | 303 | 1.8 |
| | HTF-SP1 olfactory receptor mRNA, | | | | | |
| | partial cds | | | | | |
| D28111_at | D28111 RATMAOBP2 Rat mRNA for | 796 | 860 | 210 | 220 | 3.9 |
| | MOBP (myelin-associated | | | | | |
| | oligodendrocytic basic protein), | | | | | |
| | complete cds, clone rOP1 | | | | | |
| D28560_at | D28560 RATNPHIII Rat mRNA for | 420 | 393 | 181 | 272 | −1.7 |
| | phosphodiesterase I | | | | | |
| K00512_at | K00512 rat myelin basic protein (mbp) | 3097 | 3177 | 696 | 689 | 4.5 |
| | gene mrna/cds = UNKNOWN/gb = K00512/ | | | | | |
| | gi = 205320/ug = Rn.9672/len = 1464 | | | | | |
| L13202_f_at | L13202 RATHFH2 Rattus norvegicus | 84 | 79 | 208 | 198 | 2.5 |
| | HNF-3/fork-head homolog-2 (HFH-2) | | | | | |
| | mRNA, complete cds | | | | | |
| L16532_at | L16532 Rattus norvegicus (clone | 867 | 945 | 244 | 233 | −3.8 |
| | pCNPII) 2′,3′-cyclic nucleotide 3′- | | | | | |
| | phosphodiesterase (CNPII) mRNA, | | | | | |
| | complete cds/cds = 79,1341/ | | | | | |
| | gb = L16532/gi = 294526/ | | | | | |
| | ug = Rn.2592/len = 2301 | | | | | |
| L19180_at | L19180 Rat receptor-linked protein | 331 | 429 | 46 | −13 | −1.9 |
| | tyrosine phosphatase (PTP-P1) mRNA, | | | | | |
| | complete cds/cds = 30,4517/ | | | | | |
| | gb = L19180/gi = 310201/ | | | | | |
| | ug = Rn.17237/len = 5396 | | | | | |
| M11794cds#2_f_at | M11794cds#2 RATMT12C Rat | 543 | 480 | 894 | 888 | 1.7 |
| | metallothionein-2 and metallothionein-1 | | | | | |
| | genes, complete cds | | | | | |
| M13100cds#1_g_at | M13100cds#1 RATLIN3A Rat long | 723 | 666 | 1527 | 1501 | 2.2 |
| | interspersed repetitive DNA sequence | | | | | |
| | LINE3 (L1Rn) | | | | | |
| M13100cds#1_at | M13100cds#1 RATLIN3A Rat long | 1805 | 1436 | 2832 | 2939 | 1.8 |
| | interspersed repetitive DNA sequence | | | | | |
| | LINE3 (L1Rn) | | | | | |
| M13100cds#1_g_at | M13100cds#1 RATLIN3A Rat long | 666 | 723 | 1501 | 1527 | 2.2 |
| | interspersed repetitive DNA sequence | | | | | |
| | LINE3 (L1Rn) | | | | | |
| M13100cds#5_s_at | M13100cds#5 RATLIN3A Rat long | 511 | 669 | 1125 | 1442 | 2.2 |
| | interspersed repetitive DNA sequence | | | | | |
| | LINE3 (L1Rn) | | | | | |
| M20721_f_at | M20721 RATPRPA Rat proline-rich | 129 | 100 | 282 | 281 | 2.5 |
| | protein (PRP-1) mRNA, partial cds | | | | | |
| M25888_at | M25888 Rat lipophilin mRNA, 3′ end/ | 4308 | 3199 | 1042 | 1483 | −3.0 |
| | cds = 0,520/gb = M25888/gi = 206223/ | | | | | |
| | ug = Rn.4550/len = 2585 | | | | | |
| M36317_s_at | M36317 RATTRHA Rat thyrotropin- | 116 | 132 | 298 | 310 | 2.5 |
| | releasing hormone (TRH) precursor | | | | | |
| | mRNA, complete cds | | | | | |
| M60322_at | M60322 Rat aldose reductase gene, | −111 | 168 | 562 | 464 | 2.6 |
| | complete cds/cds = 38,988/gb = M60322/ | | | | | |
| | gi = 202851/ug = Rn.2917/len = 1339 | | | | | |
| M80570_at | M80570 Rat dopamine transporter | 491 | 387 | 155 | 80 | −2.2 |
| | mRNA, complete cds/cds = 62,1921/ | | | | | |
| | gb = M80570/gi = 310097/ug = Rn.10093/ | | | | | |
| | len = 3386 | | | | | |
| Rc_AI639204_at | Rat mixed-tissue library Rattus | 309 | 311 | 484 | 606 | 1.8 |
| | norvegicus cDNA clone rx03840 3′, | | | | | |
| | mRNA sequence [Rattus norvegicus] | | | | | |
| Rc_AI639504_at | Rat mixed-tissue library Rattus | 150 | 151 | 297 | 274 | 1.9 |
| | norvegicus cDNA clone rx04791 3′, | | | | | |
| | mRNA sequence [Rattus norvegicus] | | | | | |
| Rc_AA799448_g_at | rc_AA799448 EST188945 Rattus | 410 | 386 | 197 | 171 | 2.2 |
| | norvegicus cDNA, 3′ end/ | | | | | |
| | clone = RHEAB18/clone_end = 3′/ | | | | | |
| | gb = AA799448/gi = 2862403/ | | | | | |
| | ug = Rn.8296/len = 615 | | | | | |
| Rc_AA800604_g_at | rc_AA800604 EST190101 Rattus | 119 | 232 | 396 | 413 | 1.9 |
| | norvegicus cDNA, 3′ end/clone = RLUAB65/ | | | | | |
| | clone_end = 3′/gb = AA800604/ | | | | | |
| | gi = 2863559/ug = Rn.8590/ | | | | | |
| | len = 579 | | | | | |
| Rc_AA800693_g_at | rc_AA800693 EST190190 Rattus | 749 | 985 | 553 | 441 | −1.7 |
| | norvegicus cDNA, 3′ end/ | | | | | |
| | clone = RLUAK36/clone_end = 3′/ | | | | | |
| | gb = AA800693/gi = 2863648/ | | | | | |
| | ug = Rn.6620/ len = 533 | | | | | |
| Rc_AA818072_s_at | rc_AA818072 UI-R-A0-ag-b-06-0-UI.s2 | 440 | 453 | 178 | 228 | −2.1 |
| | Rattus norvegicus cDNA, 3′ end/ | | | | | |
| | clone = UI-R-A0-ag-b-06-0-UI/ | | | | | |
| | clone_end = 3′/gb = AA818072/ | | | | | |
| | gi = 2887952/ug = Rn.11722/len = 408 | | | | | |
| Rc_AA859643_at | rc_AA859643 UI-R-E0-bs-a-08-0-UI.s1 | 404 | 520 | 193 | 215 | −2.2 |
| | Rattus norvegicus cDNA, 3′ end/ | | | | | |
| | clone = UI-R-E0-bs-a-08-0-UI/ | | | | | |
| | clone_end = 3′/gb = AA859643/ | | | | | |
| | gi = 2949163/ug = Rn.32/len = 482 | | | | | |
| Rc_AA859922_at | rc_AA859922 UI-R-E0-cg-c-04-0-UI.s1 | 344 | 413 | 712 | 615 | 1.8 |
| | Rattus norvegicus cDNA, 3′ end/ | | | | | |
| | clone = UI-R-E0-cg-c-04-0-UI/ | | | | | |
| | clone_end = 3′/gb = AA859922/ | | | | | |
| | gi = 2949442/ug = Rn.819/len = 373 | | | | | |
| Rc_AA866432_at | rc_AA866432 UI-R-E0-ch-e-06-0-UI.s1 | 628 | 537 | 302 | 251 | −2.1 |
| | Rattus norvegicus cDNA, 3′ end/ | | | | | |
| | clone = UI-R-E0-ch-e-06-0-UI/ | | | | | |
| | clone_end = 3′/gb = AA866432/ | | | | | |
| | gi = 2961893/ug = Rn.3106/len = 484 | | | | | |
| Rc_AA875411_s_at | rc_AA875411 UI-R-E0-cs-b-11-0-UI.s1 | 520 | 476 | 191 | 115 | −2.5 |
| | Rattus norvegicus cDNA, 3′ end/ | | | | | |
| | clone = UI-R-E0-cs-b-11-0-UI/ | | | | | |
| | clone_end = 3′/gb = AA875411/ | | | | | |
| | gi = 2980359/ug = Rn.2911/len = 423 | | | | | |
| Rc_AA875414_at | rc_AA875414 UI-R-E0-cs-d-07-0-UI.s1 | 218 | 193 | 549 | 634 | 2.8 |
| | Rattus norvegicus cDNA, 3′ end/ | | | | | |
| | clone = UI-R-E0-cs-d-07-0-UI/ | | | | | |
| | clone_end = 3′/gb = AA875414/ | | | | | |
| | gi = 2980362/ug = Rn.2912/len = 428 | | | | | |
| Rc_AA891940_at | rc_AA891940 EST195743 Rattus | 427 | 372 | 72 | 143 | −2.0 |
| | norvegicus cDNA, 3′ end/ | | | | | |
| | clone = RKIAI82/clone_end = 3′/ | | | | | |
| | gb = AA891940/gi = 3018819/ | | | | | |
| | ug = Rn.3508/len = 523 | | | | | |
| Rc_AA892942_at | rc_AA892942 EST196745 Rattus | 208 | 192 | 85 | 93 | 2.3 |
| | norvegicus cDNA, 3′ end/ | | | | | |
| | clone = RKIBA19/clone_end = 3′/ | | | | | |
| | gb = AA892942/gi = 3019821/ | | | | | |
| | ug = Rn.3611/len = 511 | | | | | |
| Rc_AA893593_g_at | rc_AA893593 EST197396 Rattus | 357 | 433 | 59 | −12 | −2.0 |
| | norvegicus cDNA, 3′ end/ | | | | | |
| | clone = RPLAC35/clone_end = 3′/ | | | | | |
| | gb = AA893593/gi = 3020472/ | | | | | |
| | ug = Rn.2272/len = 443 | | | | | |
| Rc_AA945589_at | rc_AA945589 EST201088 Rattus | 362 | 399 | 860 | 847 | 2.2 |
| | norvegicus cDNA, 3′ end/ | | | | | |
| | clone = RLIAP44/clone_end = 3′/ | | | | | |
| | gb = AA945589/ug = Rn.2151/ | | | | | |
| | len = 569 | | | | | |
| Rc_AA946313_s_at | rc_AA946313 EST201812 Rattus | 814 | 939 | 445 | 586 | −1.7 |
| | norvegicus cDNA, 3′ end/ | | | | | |
| | clone = RLUBD62/clone_end = 3′/ | | | | | |
| | gb = AA946313/ug = Rn.4295/ | | | | | |
| | len = 505 | | | | | |
| Rc_AI070277_s_at | rc_AI070277 UI-R-Y0-Is-h-11-0-UI.s1 | 2415 | 2557 | 1169 | 1337 | 2.0 |
| | Rattus norvegicus cDNA, 3′ end/ | | | | | |
| | clone = UI-R-Y0-Is-h-11-0-UI/ | | | | | |
| | clone_end = 3′/gb = AI070277/ | | | | | |
| | ug = Rn.4550/len = 355 | | | | | |
| Rc_AI072770_s_at | rc_AI072770 UI-R-Y0-md-g-02-0-UI.s1 | 1628 | 1426 | 327 | 343 | 4.6 |
| | Rattus norvegicus cDNA, 3′ end/ | | | | | |
| | clone = UI-R-Y0-md-g-02-0-UI/ | | | | | |
| | clone_end = 3′/gb = AI072770/ | | | | | |
| | ug = Rn.4550/len = 333 | | | | | |
| Rc_H31839_at | rc_H31839 EST106322 Rattus | 292 | 337 | 618 | 681 | 2.1 |
| | norvegicus cDNA, 3′ end/ | | | | | |
| | clone = RPCAZ43/clone_end = 3′/ | | | | | |
| | gb = H31839/gi = 977256/ | | | | | |
| | ug = Rn.14598/len = 408 | | | | | |
| U18419_at | U18419 Rattus norvegicus nonmuscle | 209 | 110 | 372 | 357 | 1.8 |
| | caldesmon mRNA, complete cds/ | | | | | |
| | cds = 723,2318/gb = U18419/ | | | | | |
| | gi = 622966/ug = Rn.10621/ | | | | | |
| | len = 5541 | | | | | |
| U31367_at | U31367 Rattus norvegicus myelin | 488 | 430 | 192 | 238 | −2.1 |
| | protein MVP17 mRNA, complete cds/ | | | | | |
| | cds = 75,536/gb = U31367/ | | | | | |
| | gi = 914967/ug = Rn.10174/len = 2268 | | | | | |
| U31866_g_at | U31866 Rattus norvegicus Nclone10 | 454 | 362 | 111 | 125 | −2.0 |
| | mRNA/cds = UNKNOWN/gb = U31866/ | | | | | |
| | gi = 1216376/ug = Rn.11164/len = 2657 | | | | | |
| U36482_g_at | U36482 Rattus norvegicus | 297 | 400 | 126 | 192 | −1.7 |
| | endoplasmic reticulum protein ERp29 | | | | | |
| | precursor, mRNA, complete cds/ | | | | | |
| | cds = 43,825/gb = U36482/gi = 2317799/ | | | | | |
| | ug = Rn.11262/ len = 1115 | | | | | |
| U37101_at | U37101 RRU37101 Rattus rattus | 167 | 85 | 403 | 436 | 2.1 |
| | granulocyte colony stimulating factor | | | | | |
| | mRNA, complete cds | | | | | |
| U50185_g_at | U50185 RNU50185 Rattus norvegicus | 249 | 182 | 446 | 345 | 1.8 |
| | kidney protein phosphatase 1 myosin | | | | | |
| | binding subunit mRNA, partial cds | | | | | |
| U89514_at | U89514 Rattus norvegicus calpain | 219 | 173 | 484 | 350 | 2.0 |
| | large subunit (nCL-4) mRNA, partial | | | | | |
| | cds/cds = 0,2024/gb = U89514/ | | | | | |
| | gi = 2358261/ug = Rn.10804/len = 2195 | | | | | |
| X05472cds#1_s_at | X05472cds#1 RNREP24R Rat 2.4 kb | 1168 | 971 | 1824 | 2461 | 2.0 |
| | repeat DNA right terminal region | | | | | |
| X58294_at | X58294 R. norvegicus mRNA for | 626 | 592 | 254 | 88 | −2.7 |
| | carbonic anhydrase II/cds = 8,790/ | | | | | |
| | gb = X58294/gi = 55837/ug = Rn.3525/ | | | | | |
| | len = 1459 | | | | | |
| X61295cds_s_at | X61295cds RNL1RTO2B R. norvegicus | 1759 | 2259 | 3785 | 4678 | 2.1 |
| | L1 retroposon, ORF2 mRNA (partial) | | | | | |
| X62086mRNA_s_at | X62086mRNA RNCYP3A1 | 562 | 538 | 254 | 236 | −2.2 |
| | R. norvegicus CYP3A1 gene for | | | | | |
| | cytochrome P450 PCN1 | | | | | |
| X69903_at | X69903 R. norvegicus mRNA for | 255 | 162 | 408 | 417 | 1.8 |
| | interleukin 4 receptor/cds = 9,2411/ | | | | | |
| | gb = X69903/gi = 56390/ug = Rn.10471/ | | | | | |
| | len = 2450 | | | | | |
| X89968_g_at | X89968 RNSNAPGEN Rattus | 928 | 1171 | 555 | 471 | −2.0 |
| | norvegicus mRNA for alpha-soluble | | | | | |
| | NSF attachment protein | | | | | |
| Y12502cds_at | Y12502cds RNFXIIIA R. norvegicus | 242 | 225 | 85 | 66 | 3.1 |
| | mRNA for factor XIIIa | | | | | |
| Y13381cds_at | Y13381cds RNAMPH1 Rattus | 93 | 73 | 266 | 240 | 3.1 |
| | norvegicus mRNA for amphiphysin, | | | | | |
| | amph1 | | | | | |

Genes that passed the filtering criteria outlined above for differential expression between 1 week withdrawal and its corresponding control in the medial prefrontal cortex (mPFC). Average difference values (from GeneChip version 3.2) are listed for each gene from all groups. Affymetrix probe set numbers are listed along with the common name of the genes, if known.

TABLE 13
VTA 1-Week Extinction to Control

| | | 1-week | 1-week | 1-week | 1-week | | | | |
|---|---|---|---|---|---|---|---|---|---|
| | | extinction | extinction | extinction | extinction | Mean | Mean | | Fold |
| Probe set no. | Description | control A | control B | B | A | control | exp | Ratio | change |
| AF078779_g_at | AF078779 Rattus | 652 | 476 | 277 | 381 | 652 | 376.5 | 0.577454 | −1.7 |
| | norvegicus putative four | | | | | | | | |
| | repeat ion channel mRNA, | | | | | | | | |
| | complete cds | | | | | | | | |
| D17614_at | D17614 Rat mRNA for 14- | 1249 | 956 | 407 | 344 | 1249 | 681.5 | 0.545637 | −1.8 |
| | 3-3 protein theta-subtype, | | | | | | | | |
| | complete cds/cds = 85,822/ | | | | | | | | |
| | gb = D17614/gi = 402508/ | | | | | | | | |
| | ug = Rn.2502/len = 2099 | | | | | | | | |
| rc_AA799299_at | rc_AA799299 EST188796 | 70 | 274 | 542 | 430 | 70 | 408 | 5.828571 | 5.8 |
| | Rattus norvegicus cDNA, 3′ | | | | | | | | |
| | end/clone = RHEAA18/ | | | | | | | | |
| | clone_end = 3′/gb = AA799299/ | | | | | | | | |
| | gi = 2862254/ug = Rn.8563/ | | | | | | | | |
| | len = 506 | | | | | | | | |
| rc_AA893191_at | rc_AA893191 EST196994 | 55 | 62 | 292 | 313 | 55 | 177 | 3.218182 | 3.2 |
| | Rattus norvegicus cDNA, 3′ | | | | | | | | |
| | end/clone = RKIBD35/ | | | | | | | | |
| | clone_end = 3′/gb = AA893191/ | | | | | | | | |
| | gi = 3020070/ug = Rn.3301/ | | | | | | | | |
| | len = 654 | | | | | | | | |
| rc_AA893327_s_at | rc_AA893327 EST197130 | 58 | 164 | 354 | 429 | 58 | 259 | 4.465517 | 4.5 |
| | Rattus norvegicus cDNA, 3′ | | | | | | | | |
| | end/clone = RKIBF13/ | | | | | | | | |
| | clone_end = 3′/gb = AA893327/ | | | | | | | | |
| | gi = 3020206/ug = Rn.2732/ | | | | | | | | |
| | len = 452 | | | | | | | | |
| rc_AA893870_at | rc_AA893870 EST197673 | 1935 | 2636 | 3948 | 4037 | 1935 | 3292 | 1.701292 | 1.7 |
| | Rattus norvegicus cDNA, 3′ | | | | | | | | |
| | end/clone = RPLAM86/ | | | | | | | | |
| | clone_end = 3′/gb = AA893870/ | | | | | | | | |
| | gi = 3020749/ug = Rn.11229/ | | | | | | | | |
| | len = 417 | | | | | | | | |
| rc_AA894330_s_at | rc_AA894330 EST198133 | 657 | 469 | 171 | 236 | 657 | 320 | 0.487062 | −2.1 |
| | Rattus norvegicus cDNA, 3′ | | | | | | | | |
| | end/clone = RSPAW76/ | | | | | | | | |
| | clone_end = 3′/gb = AA894330/ | | | | | | | | |
| | gi = 3021209/ug = Rn.122/ | | | | | | | | |
| | len = 501 | | | | | | | | |
| rc_AA944856_at | rc_AA944856 EST200355 | 489 | 381 | 187 | 206 | 489 | 284 | 0.580777 | −1.7 |
| | Rattus norvegicus cDNA, 3′ | | | | | | | | |
| | end/clone = REMAQ02/ | | | | | | | | |
| | clone_end = 3′/gb = AA944856/ | | | | | | | | |
| | gi = 3104772/ug = Rn.4992/ | | | | | | | | |
| | len = 339 | | | | | | | | |
| rc_AI137583_at | rc_AI137583 UI-R-C0-hf-a- | 603 | 482 | 246 | 226 | 603 | 364 | 0.603648 | −1.7 |
| | 03-0-UI.s1 Rattus | | | | | | | | |
| | norvegicus cDNA, 3′ end/ | | | | | | | | |
| | clone = UI-R-C0-hf-a-03-0-UI/ | | | | | | | | |
| | clone_end = 3′/gb = AI137583/ | | | | | | | | |
| | ug = Rn.3272/len = 496 | | | | | | | | |
| rc_H31887_at | rc_H31887 EST106421 | 573 | 816 | 1374 | 1058 | 573 | 1095 | 1.910995 | 1.9 |
| | Rattus norvegicus cDNA, 3′ | | | | | | | | |
| | end/clone = RPCBC38/ | | | | | | | | |
| | clone_end = 3′/gb = H31887/ | | | | | | | | |
| | gi = 977304/ug = Rn.14601/ | | | | | | | | |
| | len = 445 | | | | | | | | |
| S79214cds_s_at | S79214cds type X collagen | 457 | 395 | 136 | 267 | 457 | 265.5 | 0.580963 | −1.7 |
| | alpha 1 chain {NC1 domain} | | | | | | | | |
| | [rats, Genomic, 491 nt] | | | | | | | | |
| S81924_s_at | S81924 Otx1 = homeobox | 207 | 211 | −7 | 27 | 207 | 102 | 0.492754 | −2.0 |
| | [rats, telencephalon, mRNA | | | | | | | | |
| | Partial, 444 nt] | | | | | | | | |
| U14398_g_at | U14398 Rattus norvegicus | 518 | 483 | 93 | 171 | 518 | 288 | 0.555985 | −1.8 |
| | synaptotagmin IV homolog | | | | | | | | |
| | mRNA, complete cds/ | | | | | | | | |
| | cds = 267,1544/gb = U14398/ | | | | | | | | |
| | gi = 550453/ug = Rn.11072/ | | | | | | | | |
| | len = 2060 | | | | | | | | |
| U50842_at | U50842 RNU50842 Rattus | 415 | 376 | 102 | 177 | 415 | 239 | 0.575904 | −1.7 |
| | norvegicus ubiquitin ligase | | | | | | | | |
| | (Nedd4) protein mRNA, | | | | | | | | |
| | partial cds | | | | | | | | |
| U52663mRNA#3_s_at | U52663mRNA#3 | 475 | 361 | 181 | 202 | 475 | 271 | 0.570526 | −1.8 |
| | RATPAM27 Rattus | | | | | | | | |
| | norvegicus peptidylglycine | | | | | | | | |
| | alpha-amidating | | | | | | | | |
| | monooxygenase (PAM) | | | | | | | | |
| | gene, exon 26 | | | | | | | | |
| X57764_s_at | X57764 Rat mRNA for ET-B | 519 | 427 | 173 | 228 | 519 | 300 | 0.578035 | −1.7 |
| | endothelin receptor/ | | | | | | | | |
| | cds = 203,1528/gb = X57764/ | | | | | | | | |
| | gi = 56122/ug = Rn.11412/ | | | | | | | | |
| | len = 1892 | | | | | | | | |
| X61106cds_at | X61106cds RNORFEP | 229 | 207 | −257 | −304 | 229 | −25 | −0.10917 | 9.2 |
| | R. norvegicus ORF for P- | | | | | | | | |
| | glycoprotein (3′-most exon) | | | | | | | | |
| | containing epitope for | | | | | | | | |
| | P-glycoprotein monoclonal | | | | | | | | |
| | antibody, C219 | | | | | | | | |
| X96437mRNA_at | X96437mRNA RNPRG1 | 518 | 481 | 99 | 271 | 518 | 290 | 0.559846 | −1.8 |
| | R. norvegicus PRG1 gene | | | | | | | | |

Genes that passed the filtering criteria outlined above for differential expression between 1 week extinction and its corresponding control in the ventral tegmental area (VTA). Average difference values (from GeneChip version 3.2) are listed for each gene from all groups. Affymetrix probe set numbers are listed along with the common name of the genes, if known.

TABLE 14
VTA 1-Week Extinction to Withdrawal

| | | 1-week | 1-week | 1-week | 1-week | | | | |
|---|---|---|---|---|---|---|---|---|---|
| | | with- | with- | extinction | extinction | Mean | Mean | | Fold |
| Probe set no. | Description | drawal A | drawal B | B | A | control | exp | Ratio | change |
| AF037072_at | AF037072 Rattus | 770 | 827 | 298 | 360 | 798.5 | 329 | 0.412023 | −2.4 |
| | norvegicus carbonic | | | | | | | | |
| | anhydrase III (CA3) mRNA, | | | | | | | | |
| | complete cds/cds = 33,815/ | | | | | | | | |
| | gb = AF037072/gi = 2708635/ | | | | | | | | |
| | ug = Rn.22519/len = 1053 | | | | | | | | |
| D86711_at | D86711 D86711 Rattus | 243 | 252 | 145 | 139 | 247.5 | 142 | 0.573737 | −1.7 |
| | norvegicus cDNA/gb = D86711/ | | | | | | | | |
| | gi = 1549215/ug = Rn.4240/ | | | | | | | | |
| | len = 994 | | | | | | | | |
| D88034_at | D88034 Rattus norvegicus | 61 | 61 | 264 | 303 | 61 | 283.5 | 4.647541 | 4.6 |
| | mRNA for peptidylarginine | | | | | | | | |
| | deiminase type III, complete | | | | | | | | |
| | cds/cds = 42,2036/ | | | | | | | | |
| | gb = D88034/gi = 1644244/ | | | | | | | | |
| | ug = Rn.10658/len = 3100 | | | | | | | | |
| E02315cds_f_at | E02315cds DNA encoding | 2260 | 2240 | 799 | 1028 | 2250 | 913.5 | 0.406 | −2.5 |
| | calmodulin | | | | | | | | |
| L14323_at | L14323 Rattus norvegicus | 467 | 367 | 109 | 190 | 417 | 149.5 | 0.358513 | −2.8 |
| | phospholipase C-beta1b | | | | | | | | |
| | mRNA, complete alleles/ | | | | | | | | |
| | cds = UNKNOWN/gb = L14323/ | | | | | | | | |
| | gi = 294611/ug = Rn.9741/ | | | | | | | | |
| | len = 7203 | | | | | | | | |
| Rc_AI639465_f_at | Rat mixed-tissue library | 1172 | 999 | 466 | 341 | 1085.5 | 403.5 | 0.371718 | −2.7 |
| | Rattus norvegicus cDNA | | | | | | | | |
| | clone rx01612 3′, mRNA | | | | | | | | |
| | sequence [Rattus | | | | | | | | |
| | norvegicus] | | | | | | | | |
| Rc_AI639392_at | Rat mixed-tissue library | 264 | 247 | 84 | 78 | 255.5 | 81 | 0.317025 | −3.2 |
| | Rattus norvegicus cDNA | | | | | | | | |
| | clone rx02714 3′, mRNA | | | | | | | | |
| | sequence [Rattus | | | | | | | | |
| | norvegicus] | | | | | | | | |
| Rc_AA799410_g_at | rc_AA799410 EST188907 | −118 | −60 | 230 | 232 | −89 | 231 | −2.59551 | at least |
| | Rattus norvegicus cDNA, 3′ | | | | | | | | 2 fold |
| | end/clone = RHEAA81/ | | | | | | | | |
| | clone_end = 3′/gb = AA799410/ | | | | | | | | |
| | gi = 2862365/ug = Rn.3326/ | | | | | | | | |
| | len = 612 | | | | | | | | |
| Rc_AA894330_s_at | rc_AA894330 EST198133 | 628 | 479 | 171 | 236 | 553.5 | 203.5 | 0.36766 | −2.7 |
| | Rattus norvegicus cDNA, 3′ | | | | | | | | |
| | end/clone = RSPAW76/ | | | | | | | | |
| | clone_end = 3′/gb = AA894330/ | | | | | | | | |
| | gi = 3021209/ug = Rn.122/ | | | | | | | | |
| | len = 501 | | | | | | | | |
| Rc_AA894345_at | rc_AA894345 EST198148 | 1220 | 1203 | 2191 | 2183 | 1211.5 | 2187 | 1.8052 | 1.8 |
| | Rattus norvegicus cDNA, 3′ | | | | | | | | |
| | end/clone = RSPAZ21/ | | | | | | | | |
| | clone_end = 3′/ | | | | | | | | |
| | gb = AA894345/gi = 3021224/ | | | | | | | | |
| | ug = Rn.13530/len = 510 | | | | | | | | |
| Rc_AA899253_at | rc_AA899253 UI-R-E0-cz-g- | 1463 | 1238 | 649 | 682 | 1350.5 | 665.5 | 0.49278 | −2.0 |
| | 07-0-UI.s1 Rattus | | | | | | | | |
| | norvegicus cDNA, 3′ end/ | | | | | | | | |
| | clone = UI-R-E0-cz-g-07-0- | | | | | | | | |
| | UI/clone_end = 3′/ | | | | | | | | |
| | gb = AA899253/gi = 3034607/ | | | | | | | | |
| | ug = Rn.9560/len = 410 | | | | | | | | |
| Rc_AI010083_at | rc_AI010083 EST204534 | 1015 | 1008 | 614 | 528 | 1011.5 | 571 | 0.564508 | −1.8 |
| | Rattus norvegicus cDNA, 3′ | | | | | | | | |
| | end/clone = RLUBT52/ | | | | | | | | |
| | clone_end = 3′/gb = AI010083/ | | | | | | | | |
| | ug = Rn.2845/len = 557 | | | | | | | | |
| Rc_AI137043_at | rc_AI137043 UI-R-C2p-oj-c- | 204 | 226 | 72 | 77 | 215 | 74.5 | 0.346512 | −2.9 |
| | 01-0-UI.s1 Rattus | | | | | | | | |
| | norvegicus cDNA, 3′ end/ | | | | | | | | |
| | clone = UI-R-C2p-oj-c-01-0-UI/ | | | | | | | | |
| | clone_end = 3′/gb = AI137043/ | | | | | | | | |
| | ug = Rn.22168/len = 436 | | | | | | | | |
| Rc_AI137583_at | rc_AI137583 UI-R-C0-hf-a- | 613 | 504 | 246 | 226 | 558.5 | 236 | 0.42256 | −2.4 |
| | 03-0-UI.s1 Rattus | | | | | | | | |
| | norvegicus cDNA, 3′ end/ | | | | | | | | |
| | clone = UI-R-C0-hf-a-03-0-U1/ | | | | | | | | |
| | clone_end = 3′/gb = AI137583/ | | | | | | | | |
| | ug = Rn.3272/len = 496 | | | | | | | | |
| Rc_AI237592_at | rc_AI237592 EST234154 | 275 | 263 | 111 | 136 | 269 | 123.5 | 0.459108 | −2.2 |
| | Rattus norvegicus cDNA, 3′ | | | | | | | | |
| | end/clone = RPLDB22/ | | | | | | | | |
| | clone_end = 3′/gb = AI237592/ | | | | | | | | |
| | ug = Rn.3747/len = 592 | | | | | | | | |
| S69316_s_at | S69316 S69315S2 | 764 | 625 | 243 | 242 | 694.5 | 242.5 | 0.349172 | −2.9 |
| | GRP94/endoplasmin {5′ and | | | | | | | | |
| | 3′ regions} [rats, KNRK | | | | | | | | |
| | cells, mRNA Partial, 195 nt, | | | | | | | | |
| | segment 2 of 2] | | | | | | | | |
| AFFX_ratb2/X14115_at | X14115 Rat DNA for B2 | 212 | 231 | 43 | 56 | 221.5 | 49.5 | 0.223476 | −4.5 |
| | repeat (1-12) from gamma | | | | | | | | |
| | crystallin gene cluster. | | | | | | | | |
| X55298_at | X55298 Rat ribophorin II | 164 | 189 | 394 | 396 | 176.5 | 395 | 2.23796 | 2.2 |
| | mRNA/cds = UNKNOWN/ | | | | | | | | |
| | gb = X55298/gi = 57672/ | | | | | | | | |
| | ug = Rn.6863/len = 2234 | | | | | | | | |
| X61296cds#2_f_at | X61296cds#2 RNL1RTO2C | 543 | 574 | 63 | 263 | 558.5 | 163 | 0.291853 | −3.4 |
| | R. norvegicus L1 retroposon, | | | | | | | | |
| | ORF2 mRNA (partial) | | | | | | | | |
| X96437mRNA_at | X96437mRNA RNPRG1 | 487 | 457 | 99 | 271 | 472 | 185 | 0.391949 | −2.6 |
| | R. norvegicus PRG1 gene | | | | | | | | |
| Z21935cds_at | Z21935cds RNPROKINA | 359 | 332 | 159 | 176 | 345.5 | 167.5 | 0.484805 | −2.1 |
| | R. norvegicus protein kinase | | | | | | | | |
| | rMNK2 | | | | | | | | |

Genes that passed the filtering criteria outlined above for differential expression between 1 week withdrawal and 1 week extinction in the ventral tegmental area (VTA). Average difference values (from GeneChip version 3.2) are listed for each gene from all groups. Affymetrix probe set numbers are listed along with the common name of the genes, if known.

TABLE 15
VTA 1-Week Withdrawal to Control

| | | R3KJF020 | R3KJF020 | R3KJF020 | R3KJF020 | Mean | Mean | | Fold |
|---|---|---|---|---|---|---|---|---|---|
| Probe set no. | Description | 12264VT | 12263VT | 12261VT | 12262VT | control | exp | Ratio | change |
| AA799389_g_at | AA799389 EST188886 | 398 | 327 | 661 | 694 | 362.5 | 677.5 | 1.868966 | 1.9 |
| | Rattus norvegicus cDNA, 5′ | | | | | | | | |
| | end/clone = RHEAA70/ | | | | | | | | |
| | clone_end = 5′/gb = AA799389/ | | | | | | | | |
| | gi = 2862344/ug = Rn.3788/ | | | | | | | | |
| | len = 588 | | | | | | | | |
| AF015305_at | AF015305 Rattus | 251 | 358 | 590 | 663 | 304.5 | 626.5 | 2.057471 | 2.1 |
| | norvegicus equilbrative | | | | | | | | |
| | nitrobenzylthioinosine- | | | | | | | | |
| | insensitive nucleoslde | | | | | | | | |
| | transporter mRNA, | | | | | | | | |
| | complete cds/cds = 157,1527/ | | | | | | | | |
| | gb = AF015305/gi = 2656138/ | | | | | | | | |
| | ug = Rn.7203/len = 1678 | | | | | | | | |
| AF064868_g_at | AF064868 Rattus | −125 | −223 | 465 | 487 | −174 | 476 | −2.73563 | at least |
| | norvegicus brain-enriched | | | | | | | | 2 fold |
| | guanylate kinase- | | | | | | | | |
| | associated protein 1 mRNA, | | | | | | | | |
| | complete cds | | | | | | | | |
| AF079162_at | AF079162 Rattus | 124 | 102 | 369 | 471 | 113 | 420 | 3.716814 | 3.7 |
| | norvegicus patched (ptc) | | | | | | | | |
| | mRNA, partial cds | | | | | | | | |
| D84667_at | D84667 Rattus norvegicus | 355 | 430 | 260 | 113 | 392.5 | 186.5 | 0.475159 | −2.1 |
| | mRNA for phosphatidy- | | | | | | | | |
| | linositol 4-kinase, complete | | | | | | | | |
| | cds | | | | | | | | |
| J03179_at | J03179 Rat D-binding | 252 | 261 | 152 | 142 | 256.5 | 147 | 0.573099 | −1.7 |
| | protein mRNA, complete | | | | | | | | |
| | cds/cds = 367,1344/ | | | | | | | | |
| | gb = J03179/gi = 203942/ | | | | | | | | |
| | ug = Rn.11274/len = 1622 | | | | | | | | |
| J03886_at | J03886 Rat skeletal muscle | 670 | 891 | 1565 | 1194 | 780.5 | 1379.5 | 1.767457 | 1.8 |
| | myosin light chain kinase, | | | | | | | | |
| | complete cds/cds = 59,1891/ | | | | | | | | |
| | gb = J03886/gi = 205496/ | | | | | | | | |
| | ug = Rn.9685/len = 2799 | | | | | | | | |
| K00750exon#2-3_at | K00750exon#2-3 RATCYC | 903 | 713 | 435 | 418 | 808 | 426.5 | 0.527847 | −1.9 |
| | Rat (Sprague-Dawley) | | | | | | | | |
| | cytochrome c nuclear- | | | | | | | | |
| | encoded mitochondrial | | | | | | | | |
| | gene and flanks | | | | | | | | |
| L07925_g_at | L07925 RATGNDSA Rattus | 224 | 168 | 495 | 413 | 196 | 454 | 2.316327 | 2.3 |
| | rattus guanine nucleotide | | | | | | | | |
| | dissociation stimulator for a | | | | | | | | |
| | ras-related GTPase mRNA, | | | | | | | | |
| | complete cds | | | | | | | | |
| M33962_g_at | M33962 Rat protein- | 201 | 246 | 420 | 391 | 223.5 | 405.5 | 1.814318 | 1.8 |
| | tyrosine-phospatase | | | | | | | | |
| | (PTPase) mRNA, complete | | | | | | | | |
| | cds/cds = 119,1417/ | | | | | | | | |
| | gb = M33962/gi = 206496/ | | | | | | | | |
| | ug = Rn.11317/len = 4127 | | | | | | | | |
| M94918mRNA_f_at | M94918mRNA | 1873 | 1372 | 3852 | 3114 | 1622.5 | 3483 | 2.146687 | 2.1 |
| | RATBETGLOX Rat beta- | | | | | | | | |
| | globin gene, exons 1-3 | | | | | | | | |
| rc_AI639204_at | Rat mixed-tissue library | 247 | 175 | 453 | 331 | 211 | 392 | 1.85782 | 1.9 |
| | Rattus norvegicus cDNA | | | | | | | | |
| | clone rx03840 3′, mRNA | | | | | | | | |
| | sequence [Rattus | | | | | | | | |
| | norvegicus] | | | | | | | | |
| rc_AA799571_at | rc_AA799571 EST189068 | 510 | 400 | 105 | 253 | 455 | 179 | 0.393407 | −2.5 |
| | Rattus norvegicus cDNA, 3′ | | | | | | | | |
| | end/clone = RHEAC67/ | | | | | | | | |
| | clone_end = 3′/gb = AA799571/ | | | | | | | | |
| | gi = 2862526/ug = Rn.3458/ | | | | | | | | |
| | len = 541 | | | | | | | | |
| rc_AA892154_g_at | rc_AA892154 EST195957 | 224 | 240 | 75 | 54 | 232 | 64.5 | 0.278017 | −3.6 |
| | Rattus norvegicus cDNA, 3′ | | | | | | | | |
| | end/clone = RKIAN02/ | | | | | | | | |
| | clone_end = 3′/gb = AA892154/ | | | | | | | | |
| | gi = 3019033/ug = Rn.3279/ | | | | | | | | |
| | len = 386 | | | | | | | | |
| rc_AA956149_at | rc_AA956149 UI-R-E1-fg-b- | 239 | 206 | 570 | 752 | 222.5 | 661 | 2.970787 | 3.0 |
| | 03-0-UI.s2 Rattus | | | | | | | | |
| | norvegicus cDNA, 3′ end/ | | | | | | | | |
| | clone = UI-R-E1-fg-b-03-0-U1/ | | | | | | | | |
| | clone_end = 3′/gb = AA956149/ | | | | | | | | |
| | ug = Rn.8930/len = 471 | | | | | | | | |
| rc_AI179445_at | rc_AI179445 EST223155 | 248 | 230 | 119 | 127 | 239 | 123 | 0.514644 | −1.9 |
| | Rattus norvegicus cDNA, 3′ | | | | | | | | |
| | end/clone = RSPCH43/ | | | | | | | | |
| | clone_end = 3′/gb = AI179445/ | | | | | | | | |
| | ug = Rn.221/len = 438 | | | | | | | | |
| S61973_at | S61973 NMDA receptor | 2493 | 1996 | 1104 | 1477 | 2244.5 | 1290.5 | 0.574961 | −1.7 |
| | glutamate-binding subunit | | | | | | | | |
| | [rats, mRNA, 1742 nt] | | | | | | | | |
| S72637_s_at | S72637 tumor-suppressive | 279 | 202 | 494 | 433 | 240.5 | 463.5 | 1.927235 | 1.9 |
| | gene [rats, RSV-trans- | | | | | | | | |
| | formed 3Y1 fibroblast cells, | | | | | | | | |
| | SR-3Y1, mRNA, 1788 nt] | | | | | | | | |
| U21720mRNA_at | U21720mRNA RNU21720 | 276 | 369 | 564 | 576 | 322.5 | 570 | 1.767442 | 1.8 |
| | Rattus norvegicus clone | | | | | | | | |
| | C201 intestinal epithelium | | | | | | | | |
| | proliferating cell-associated | | | | | | | | |
| | mRNA sequence | | | | | | | | |
| U88036_at | U88036 Rattus norvegicus | 443 | 415 | 214 | 278 | 429 | 246 | 0.573427 | −1.7 |
| | brain digoxin carrier protein | | | | | | | | |
| | mRNA, complete cds/ | | | | | | | | |
| | cds = 118,2103/gb = U88036/ | | | | | | | | |
| | gi = 2501807/ug = Rn.5641/ | | | | | | | | |
| | len = 3622 | | | | | | | | |
| X04070_at | X04070 Rat liver mRNA for | 296 | 279 | 788 | 683 | 287.5 | 735.5 | 2.558261 | 2.6 |
| | gap junction protein/ | | | | | | | | |
| | cds = 31,882/gb = X04070/ | | | | | | | | |
| | gi = 56205/ug = Rn.10444/ | | | | | | | | |
| | len = 1485 | | | | | | | | |
| X60351cds_s_at | X60351cds RRLENSABC | 295 | 227 | 516 | 417 | 261 | 466.5 | 1.787356 | 1.8 |
| | R. rattus mRNA for alpha B- | | | | | | | | |
| | crystallin (ocular lens tissue) | | | | | | | | |
| X61106cds_at | X61106cds RNORFEP | −1 | 6 | 262 | 277 | 2.5 | 269.5 | 107.8 | at least |
| | R. norvegicus ORF for P- | | | | | | | | 2 fold |
| | glycoprotein (3′-most exon) | | | | | | | | |
| | containing epitope for | | | | | | | | |
| | P-glycoprotein monoclonal | | | | | | | | |
| | antibody, C219 | | | | | | | | |
| X70667cds_at | X70667cds RRMC3RA | 239 | 295 | 432 | 555 | 267 | 493.5 | 1.848315 | 1.8 |
| | R. rattus mRNA for | | | | | | | | |
| | melanocortin-3 receptor | | | | | | | | |

Genes that passed the filtering criteria outlined above for differential expression between 1 week withdrawal and its corresponding control in the ventral tegmental area (VTA). Average difference values (from GeneChip version 3.2) are listed for each gene from all groups. Affymetrix probe set numbers are listed along with the common name of the genes, if known.

TABLE 16
Genes that are differentially regulated in various brain
regions in response to extinction and withdrawal
Brain Region

| | |
|---|---|
| Nac core | |
| Affymetrix Probe Set # | 1 week withdrawal to control |
| | Gene Name |
| X56729mRNA_at | calpastatin |
| | 1 week extinction to control |
| | Gene Name |
| K02248cds_s_at | somatostatin-14 |
| Z11581_at | kainate receptor subunit (ka2) |
| | 1 week extinction to withdrawal |
| | Gene Name |
| M25890_at | melanocortin-3 receptor |
| M92076_at | somatostatin |
| X06564_at | metabotropic glutamate receptor 3 |
| AF102855_at | NCAM |
| | synaptic SAPAP-interacting protein |
| CeA | |
| | 1 week withdrawal to control |
| | Gene Name |
| AB016160_g_at | GABAB receptor 1c |
| D83538_g_at | phosphatidylinositol 4-kinase |
| | 1 week extinction to control |
| | Gene Name |
| AB016161cds_i_at | GABAB receptor 1d |
| AF042830_at | tyrosine kinase receptor Ret (c-ret) |
| E13644cds_s_at | Neurodap-1 |
| | 1 week extinction to withdrawal |
| | Gene Name |
| M32754cds_s_at | inhibin alpha-subunit |
| U14192complete_seq_at | vesicular transport factor |
| VTA | |
| | 1 week withdrawal to control |
| | Gene Name |
| D84667_at | phosphatidylinositol 4-kinase |
| M33962_g_at | protein-tyrosine-phospatase (PTPase) |
| S61973_at | NMDA receptor glutamate-binding subunit |
| X70667cds_at | melanocortin-3 receptor |
| | 1 week extinction to control |
| | Gene Name |
| U14398_g_at | synaptotagmin IV homolog |
| | 1 week extinction to withdrawal |
| | Gene Name |
| E02315cds_f_at | calmodulin |
| Z21935cds_at | protein kinase rMNK2 |
| L14323_at | phospholipase C-beta1b |
| Frontal Cortex | |
| | 1 week withdrawal to control |
| | Gene Name |
| D28560_at | phosphodiesterase I |
| L19180_at | tyrosine phosphatase (PTP-P1) |
| M80570_at | dopamine transporter |
| | 1 week extinction to control |
| | Gene Name |
| D30040_at | RAC protein kinase alpha |
| K01701_at | oxytocin/neurophysin |
| M32061_at | alpha-2B-adrenergic receptor |
| U56261_s_at | SNAP-25a |
| | 1 week extinction to withdrawal |
| | Gene Name |
| D13212_s_at | NMDAR2C |
| K01701_at | oxytocin/neurophysin |
| U92284_at | GABA-A receptor epsilon |
| Nac shell | |
| AJ011318.1 | GABA-B receptor subunit gb2 |
| X87900.1 | Myelin-associated basic protein |
| L13041.1 | Calcitonin receptor |
| U92535.1 | Bos taurus-like neuronal axonal protein |
| X98051.1 | FRA-2 |
| AI009098 | Similar to human oxygen regulated |
| | protein |
| AI014091 | Similar to mouse mrg1 protein |
| U18772 | Pentraxin |
| U03414 | Olfactomedin related protein |
| U19866.1 | Arc - growth factor enriched in dendrites |
| U28938 | Protein tyrosine phosphatase |
| U67863.1 | Melanocortin 4 receptor |
| U69702.1 | ALK-7 kinase |
| U88958.1 | Neuritin |
| X55812.1 | CB1 cannabinoid receptor |

### Example 2

### Analysis of Western Blots

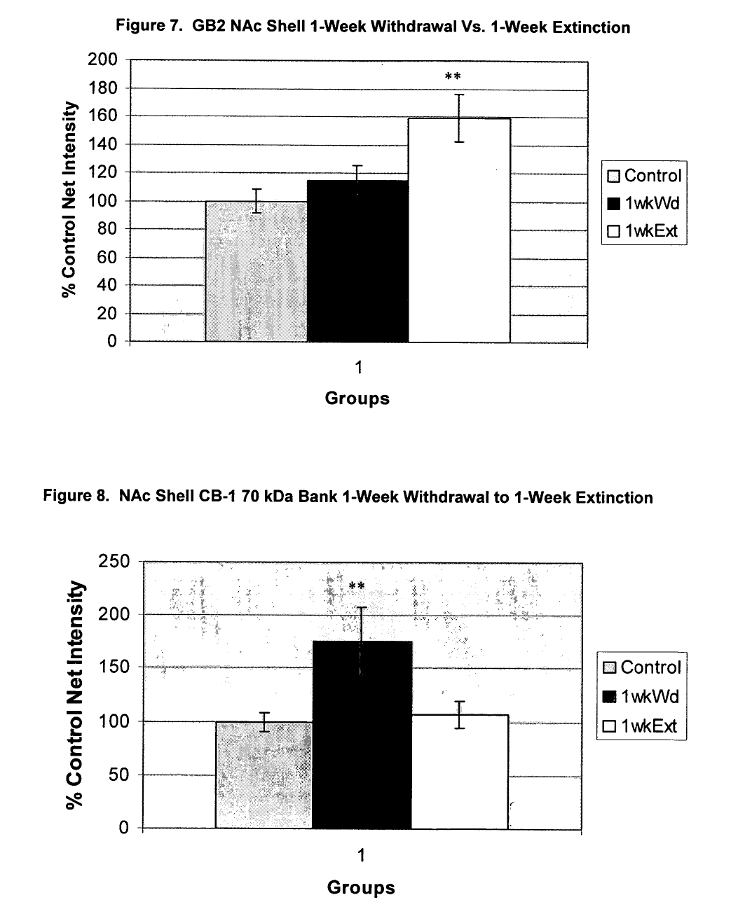

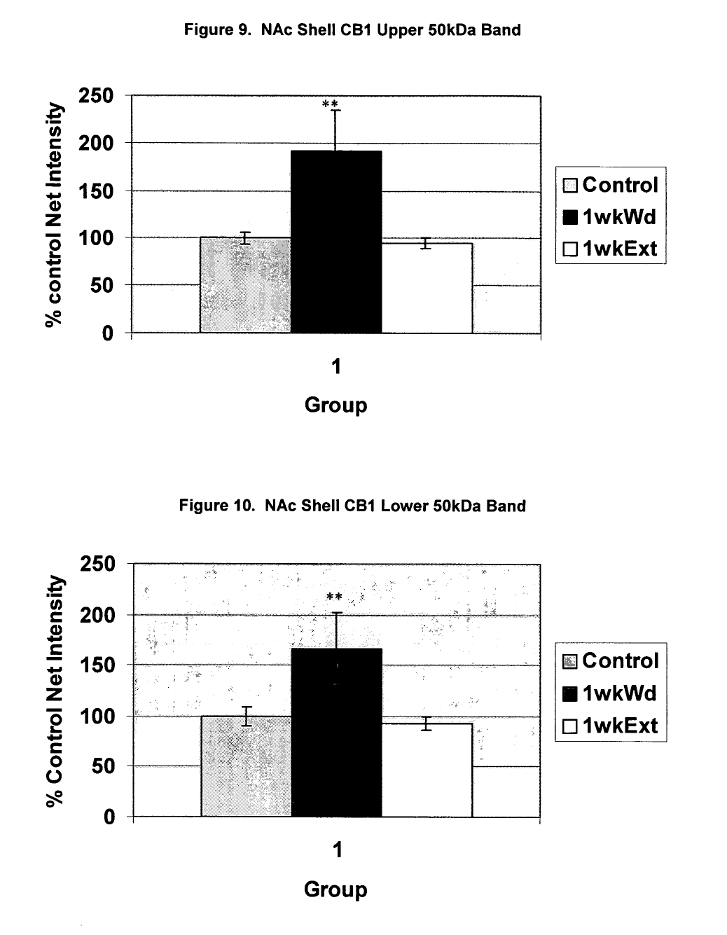

FIG. 7 demonstrates that protein levels of gb2 are increased in the nucleus accumbens shell of the 1 week extinction group compared to control animals. This result supports the microarray results and gives stronger evidence for the role of this protein in drug-seeking. In contrast CB-1 protein levels are increased in the nucleus accumbens of the 1 week withdrawal group compared to controls (FIGS. 8-10), though the microarray results showed a decrease. Nevertheless, the results suggest an important role for CB-1 in drug-seeking.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A method of inhibiting addiction-related behavior in a subject suffering from cocaine addiction, the method comprising administering to the subject a therapeutically effective amount of a therapeutic agent which has the ability to modulate the level of activity of a polypeptide encoded by at least one gene identified in one or more of Tables 1-15.

2. The method of claim 1, wherein the therapeutic agent modulates the level of transcription or translation of the gene.

3. The method of claim 1, wherein the therapeutic agent modulates the enzymatic activity of a polypeptide encoded by the gene.

4. The method of claim 1, wherein the gene is identified in one or more of Tables 1, 5, 8, 11 and 14.

5. The method of claim 1, wherein the gene is identified in Table 1.

6. The method of claim 5, wherein the at least one gene identified in Table 1 encodes a polypeptide selected from the group consisting of GABA-B receptor subunit gb2, myelin-associated basic protein, calcitonin receptor, Bos taurus-like neuronal axonal protein, FRA-2, similar to human oxygen-regulated protein, similar to mouse mrg 1 protein, pentraxin, olfactomedin-related protein, arc-growth factor enriched in dendrites, protein tyrosine phosphatase, melanocortin 4 receptor, ALK-7 kinase, neuritin and CB1 cannabinoid receptor.

7. The method of claim 6, wherein the polypeptide is GABA-B receptor subunit gb2, FRA-2 or CB1 cannabinoid receptor.

8. The method of claim 1, wherein the gene is identified in Table 2.

9. The method of claim 1, wherein the gene is identified in Table 4.

10. The method of claim 9, wherein the gene identified in Table 4 encodes a polypeptide selected from the group consisting of GABAB receptor 1d, tyrosine kinase receptor RET and Neurodap-1.

11. The method of claim 1, wherein the gene is identified in Table 5.

12. The method of claim 11, wherein the gene identified in Table 5 encodes a polypeptide selected from the group consisting of inhibin alpha-subunit and vesicular transport factor.

13. The method of claim 1, wherein the gene is identified in Table 6.

14. The method of claim 13, wherein the gene identified in Table 6 encodes a polypeptide selected from the group consisting of GABAB receptor 1c and phosphatidylinositol 4-kinase.

15. The method of claim 1, wherein the gene is identified in Table 7.

16. The method of claim 15, wherein the gene identified in Table 7 encodes a polypeptide selected from the group consisting of somatostain-14 and kainate receptor submit (ka2).

17. The method of claim 1, wherein the at least one gene is identified in Table 8.

18. The method of claim 17, wherein the at least one gene identified in Table 8 encodes a polypeptide selected from the group consisting of melanocortin-3 receptor, somatostatin, metabotropic glutamate receptor 3, NCAM polypeptide and synaptic SAPAP-interacting protein.

19. The method of claim 1, wherein the at least one gene is identified in Table 9.

20. The method of claim 19, wherein the at least one gene identified in Table 9 is calpastatin.

21. The method of claim 1, wherein the at least one gene is identified in Table 10.

22. The method of claim 21, wherein the at least one gene identified in Table 10 encodes a polypeptide selected from the group consisting of RAC protein kinase alpha, alpha-2B-adrenergic receptor and SNAP-25A.

23. The method of claim 1, wherein the at least one gene is identified in Table 11.

24. The method of claim 23, wherein the at least one gene identified in Table 11 encodes a polypeptide selected from the group consisting of oxytosin/neurophysin, NMDAR2C and GABA-A receptor epsilon.

25. The method of claim 1, wherein the at least one gene is identified in Table 12.

26. The method of claim 25, wherein the at least one gene identified in Table 12 encodes a polypeptide selected from the group consisting of phosphodiesterase I, tyrosine phosphatase and dopamine transporter.

27. The method of claim 1, wherein the at least one gene is identified in Table 13.

28. The method of claim 27, wherein the at least one gene identified in Table 13 encodes synaptotagmin IV homolog.

29. The method of claim 1, wherein the at least one gene is identified in Table 14.

30. The method of claim 29, wherein the at least one gene identified in Table 14 encodes a polypeptide selected from the group consisting of calmodulin, protein kinase rMNK2 and phospholipase C-beta1b.

31. The method of claim 1, wherein the at least one gene is identified in Table 15.

32. The method of claim 31, wherein the at least one gene identified in Table 15 encodes a polypeptide selected from the group consisting of phosphatidylinositol 4-kinase and protein-tyrosine-phosphatase.

33. The method of claim 1, wherein the therapeutic agent is selected from the group consisting of an antisense sequence, a ribozyme, a double stranded RNA, an antagonist and an agonist.

34. The method of claim 1, wherein the cocaine-addiction related behavior is cocaine seeking.

35. A method of inhibiting addiction-related behavior in a subject suffering from cocaine addiction, the method comprising administering to the subject a therapeutically effective amount of a therapeutic agent which has the ability to decrease transcription/translation of, or decrease the activity of a protein encoded by, at least one gene that encodes a polypeptide selected from the group consisting of hypertension-regulated vascular factor, myelin-associated basic protein, PB cadherin, calcitonin receptor, melanocortin 4 receptor, ALK-7 kinase, and retroposon.

36. The method of claim 35, wherein the therapeutic agent is selected from the group consisting of an antisense sequence, a ribozyme, a double stranded RNA, an antagonist and an agonist.

37. A method of inhibiting addiction-related behavior in a subject suffering from cocaine addiction, the method comprising administering to the subject a therapeutically effective amount of an agonist that activates a protein selected from the group consisting of GABA-B receptor subunit gb2, cell adhesion-like molecule, bos taurus-like neuronal axonal protein, a polypeptide similar to mouse chemokine-like factor, FRA-2, a protein similar to human oxygen-regulated protein, a protein similar to mouse mrg1 protein, pentraxin, malic enzyme, olfactomedin-related protein, arc-growth factor, protein tyrosine phosphatase, krox, neuritin, microtubule-associated protein 2d, and CB1 cannabinoid receptor.

38. A method for identifying an agent to be tested for an ability to prevent or inhibit cocaine addiction-related behavior, the method comprising:
a) combining in a reaction mixture a candidate agent with a protein encoded by a gene identified in Tables 1-15; and
b) determining whether the candidate agent binds to and/or modulates activity of the protein.

39. The method of claim 38, wherein the protein is selected from the group consisting of hypertension-regulated vascular factor, myelin-associated basic protein, PB cadherin, calcitonin receptor, ALK-7 kinase and retroposon, cell adhesion-like molecule, bos taurus-like neuronal axonal protein, a polypeptide similar to mouse chemokine-like factor, FRA-2, a polypeptide similar to human oxygen-regulated protein, a polypeptide similar to mouse mrg1 protein, pentraxin, malic enzyme, olfactomedin-related protein, arc-growth factor, protein tyrosine phosphatase, krox, neuritin and microtubule-associated protein.

40. The method of claim 38, further comprising adding to the reaction mixture a competitor molecule that competes with binding of the candidate agent to the protein, either prior to or subsequent to combining the protein with the candidate agent.

41. The method of claim 38, wherein the reaction mixture is a cell-free protein mixture.

42. The method of claim 38, wherein the reaction mixture comprises a cell membrane preparation.

43. The method of claim 38, wherein the reaction mixture comprises a cell that comprises a heterologous gene that encodes the protein.

44. The method of claim 38, wherein (b) comprises determining a change in the level of an intracellular second messenger responsive to signaling by the protein.

45. The method of claim 38, wherein (b) comprises detecting a change in the level of expression of a reporter gene operatively linked to a transcriptional control sequence.

46. The method of claim 45, wherein the reporter gene encodes a protein selected from the group consisting of luciferase, alkaline phosphatase, chloramphenicol acetyl transferase and β-galactosidase.

47. The method of claim 38, wherein the method further comprises:
c) administering the candidate agent identified in b) to a cocaine-addicted subject or brain cells of a cocaine-addicted subject, wherein the cocaine-addicted subject is undergoing withdrawal; and
d) determining a level of expression of at least one gene identified in Tables 1-15 in brain cells of the cocaine-addicted subject, and comparing the level of expression to that observed in brain cells of a cocaine-addicted subject to which the candidate agent is not administered, wherein a change in expression level is indicative of the candidate having efficacy in preventing or inhibiting cocaine addiction-related behavior.

48. The method of claim 38, wherein the method further comprises:
c) administering the candidate agent identified in b) to a cocaine-addicted subject that is undergoing withdrawal; and
d) determining whether the withdrawal symptoms exhibited by the subject are reduced upon administration of the candidate agent.

49. A method for identifying an agent to be tested for an ability to prevent or inhibit addiction related behavior, the method comprising:
a) exposing a cocaine-addicted subject or brain cells of a cocaine-addicted subject to a candidate agent, wherein the cocaine-addicted subject is undergoing withdrawal;
b) determining a level of expression of at least one gene in the cocaine-addicted subject or brain cells of the cocaine-addicted subject, wherein the at least one gene is identified in Tables 1-15; and
c) comparing the level of expression of the gene in the cocaine-addicted subject or brain cells of the cocaine-addicted subject in the presence of the candidate agent with the level of expression of the gene in the cocaine-addicted subject or brain cells of the cocaine-addicted subject in the absence of the candidate agent, wherein a reversal in the level of expression of the gene in cocaine-addicted subject or brain cells of the cocaine addicted subject in the presence of the candidate agent relative to the level of expression of the gene in the absence of the candidate agent indicates that the candidate agent is an agent to be tested for the ability to prevent or inhibit addiction related behavior.

50. The method of claim 49, wherein the at least one gene is identified in Tables 1, 5, 8, 11 and 14.

51. The method of claim 49, wherein the at least one gene is identified in Table 1.

52. The method of claim 51, wherein the at least one gene identified in Table 1 encodes a polypeptide selected from the group consisting of GABA-B receptor subunit gb2, myelin-associated basic protein, calcitonin receptor, Bos taurus-like neuronal axonal protein, FRA-2, a polypeptide similar to human oxygen-regulated protein, a polypeptide similar to mouse mrg 1 protein, pentraxin, olfactomedin-related protein, arc-growth factor enriched in dendrites, protein tyrosine phosphatase, melanocortin 4 receptor, ALK-7 kinase, neuritin and CB1 cannabinoid receptor.

53. The method of claim 52, wherein the at least one gene encodes a polypeptide selected from the group consisting of GABA-B receptor subunit gb2, FRA-2 and CB1 cannabinoid receptor.

54. The method of claim 49, wherein the at least one gene is identified in Table 2.

55. The method of claim 49, wherein the at least one gene is identified in Table 4.

56. The method of claim 55, wherein the at least one gene identified in Table 4 encodes a polypeptide selected from the group consisting of GABAB receptor 1d, tyrosine kinase receptor RET and Neurodap-1.

57. The method of claim 49, wherein the at least one gene is identified in Table 5.

58. The method of claim 57, wherein the at least one gene identified in Table 5 encodes a polypeptide selected from the group consisting of inhibin alpha-subunit and vesicular transport factor.

59. The method of claim 49, wherein the at least one gene is identified in Table 6.

60. The method of claim 59, wherein the at least one gene identified in Table 6 encodes a polypeptide selected from the group consisting of GABAB receptor 1c and phosphatidylinositol 4-kinase.

61. The method of claim 49, wherein the at least one gene is identified in Table 7.

62. The method of claim 61, wherein the at least one gene identified in Table 7 encodes a polypeptide selected from the group consisting of somatostain-14 and kainate receptor submit (ka2).

63. The method of claim 49, wherein the at least one gene is identified in Table 8.

64. The method of claim 63, wherein the at least one gene identified in Table 8 encodes a polypeptide selected from the group consisting of melanocortin-3 receptor, somatostatin, metabotropic glutamate receptor 3, NCAM polypeptide and synaptic SAPAP-interacting protein.

65. The method of claim 49, wherein the at least one gene is identified in Table 9.

66. The method of claim 65, wherein the at least one gene identified in Table 9 encodes calpastatin.

67. The method of claim 49, wherein the at least one gene is identified in Table 10.

68. The method of claim 67, wherein the at least one gene identified in Table 10 encodes a polypeptide selected from the group consisting of RAC protein kinase alpha, alpha-2B-adrenergic receptor and SNAP-25A.

69. The method of claim 49, wherein the at least one gene is identified in Table 11.

70. The method of claim 69, wherein the at least one gene identified in Table 11 encodes a polypeptide selected from the group consisting of oxytosin/neurophysin, NMDAR2C and GABA-A receptor epsilon.

71. The method of claim 49, wherein the at least one gene is identified in Table 12.

72. The method of claim 71, wherein the at least one gene identified in Table 12 encodes a polypeptide selected from the group consisting of phosphodiesterase I, tyrosine phosphatase and dopamine transporter.

73. The method of claim 49, wherein the at least one gene is identified in Table 13.

74. The method of claim 73, wherein the at least one gene identified in Table 13 encodes synaptotagmin IV homolog.

75. The method of claim 49, wherein the at least one gene is identified in Table 14.

76. The method of claim 75, wherein the at least one gene identified in Table 14 encodes a polypeptide selected from the group consisting of calmodulin, protein kinase rMNK2, phospholipase C-beta1b.

77. The method of claim 49, wherein the at least one gene is identified in Table 15.

78. The method of claim 77, wherein the at least one gene identified in Table 15 encodes a polypeptide selected from the group consisting of phosphatidylinositol 4-kinase and protein-tyrosine-phosphatase.

79. The method of claim 49, wherein the cocaine addiction-related behavior is cocaine craving.

80. The method of claim 49, wherein the level of expression of the gene is determined by detecting the level of expression of a protein encoded by the gene.

81. The method of claim 80, wherein the level of expression of the protein encoded by the gene is detected through western blotting by utilizing a labeled probe specific for the protein.

82. The method of claim 81, wherein the labeled probe is an antibody.

83. The method of claim 82, wherein the antibody is a monoclonal antibody.

84. The method of claim 49, wherein the level of expression of at least two or more genes in the sample is detected in (b).

85. The method of claim 49, wherein the level of expression of the gene is determined by detecting the level of expression of a mRNA corresponding to the gene.

86. The method of claim 85, wherein the level of expression of mRNA is detected by techniques selected from the group consisting of Northern blot analysis, reverse transcription PCR, real time quantitative PCR and microarray analysis.

87. The method of claim 49, wherein the agent is selected from the group consisting of antisense nucleotides, ribozymes and double-stranded RNAs.

88. A method for identifying an agent to be tested for an ability to prevent or inhibit cocaine addiction-related behavior, the method comprising:
a) contacting a brain tissue sample from each of a subject having a cocaine addiction-related behavior and a cocaine addiction-free subject;
b) detecting a level of expression of at least one gene in both tissue samples, wherein the gene encodes a polypeptide selected from the group consisting of hypertension-regulated vascular factor, myelin-associated basic protein, PB cadherin, calcitonin receptor, melanocortin 4 receptor, ALK-7 kinase and retroposon.
c) subtracting the level of expression of the gene in the sample obtained from the cocaine addiction-free subject from the level of expression of the gene in the sample obtained from the subject having cocaine addiction-related behavior to provide a first value;
d) administering a candidate agent to each of a subject having a cocaine addiction-related behavior and a cocaine addiction-free subject;
e) detecting a level of expression of at least one gene in both tissue samples obtained from the subjects treated with the candidate agent;
f) subtracting the level of expression of the at least one gene in the sample obtained from the treated cocaine addiction-free subject from the level of expression of the gene in the sample obtained from the treated subject having the cocaine addiction-related behavior to provide a second value; and
g) comparing the second value with the first value wherein a decreased second value relative to the first value is indicative of an agent to be tested for an ability to prevent or inhibit cocaine addiction-related behavior.

89. A method for identifying agents to be tested for an ability to prevent or inhibit cocaine addiction-related behavior, the method comprising:
a) obtaining a brain tissue sample from each of a subject having a cocaine addiction-related behavior and a cocaine addiction-free subject;
b) detecting a level of expression of at least one gene in both tissue samples, wherein the gene encodes a polypeptide selected from the group consisting of GABA-B receptor subunit gb2, cell adhesion-like molecule, bos taurus-like neuronal axonal protein, similar to mouse chemokine-like factor, FRA-2, a polypeptide similar to human oxygen-regulated protein, a polypeptide similar to mouse mrg1 protein, pentraxin, malic enzyme, olfactomedin-related protein, arc-growth factor enriched in dendrites, protein tyro sine phosphatase, krox, neuritin, microtubule-associated protein 2d and CB1 cannabinoid receptor;
c) subtracting the level of expression of the gene in the sample obtained from the cocaine addiction-free subject from the level of expression of the gene of the sample obtained from the subject having cocaine addiction-related behavior to provide a first value;
d) administering a candidate agent to each of a subject having a cocaine addiction-related behavior and a cocaine addiction-free subject;
e) detecting a level of expression of the gene in both tissue samples obtained from the subjects treated with the candidate agent;
f) subtracting the level of expression of the gene in the sample obtained from the treated cocaine addiction-free subject from the level of expression of the gene in the sample obtained from the treated subject having the cocaine addiction-related behavior to provide a second value; and
g) comparing the second value with the first value wherein an increased second value relative to the first value is indicative of an agent to be tested for an abilty to prevent or inhibit cocaine addiction related behavior.
